(19) United States
(12) Patent Application Publication
(10) Pub. No.: US 2012/0255040 A1
(43) Pub. Date: Oct. 4, 2012

(54) Cell Model and Methods Using the Same

(76) Inventors: Kai Dittmann, Goettingen (DE); Juergen Wienands, Bovenden (DE)

(21) Appl. No.: 13/075,846

(22) Filed: Mar. 30, 2011

(51) Int. Cl. G01N 33/00; A01K 67/027; C12N 5/10

(52) U.S. Cl. 800/3; 435/354; 800/18; 800/13; 800/14; 800/9; 800/10

(57) ABSTRACT

The present invention relates in a first aspect to a cell model containing chondrocytes whereby said chondrocytes contain a first heterologous nucleic acid sequence operably linked with a mb1 promoter sequence. In another aspect, the present invention relates to a cell model, in particular, to a transgenic animal model whose genome comprises a first heterologous nucleic acid sequence encoding a recombinase and/or restriction enzyme operably linked to a chondrocyte specific promoter, and a second heterologous nucleic acid sequence encoding a target peptide of interest wherein the second nucleic acid sequence further comprises recombination sequences or restriction site for the enzyme encoded by the first heterologous nucleic acid sequence. In addition, methods for screening foreign agent or methods for testing the efficacy and/or efficiency of an agent are provided. Said agents are tested for their ability to reduce or prevent or to allow treatment of diseases, disorders or conditions involving arthropathy and/or chondropathy, in particular, spinal malfunction.

The present invention relates in a first aspect to a cell model containing chondrocytes whereby said chondrocytes contain a first heterologous nucleic acid sequence operably linked with a mb1 promoter sequence. In another aspect, the present invention relates to a cell model, in particular, to a transgenic animal model whose genome comprises a first heterologous nucleic acid sequence encoding a recombinase and/or restriction enzyme operably linked to a chondrocyte specific promoter, and a second heterologous nucleic acid sequence encoding a target of interest, like a peptide of interest, whereby the second nucleic acid sequence further comprises recombination sequences or restriction site(s) for the enzyme encoded by the first heterologous nucleic acid sequence. In addition, methods of screening for an agent or methods of testing for the efficacy and/or efficiency of an agent are provided. Said agents are tested for their ability to reduce or prevent or to allow treatment of diseases, disorders or conditions involving arthropathy and/or chondropathy, in particular, spinal malfunction.

### BACKGROUND ART

During vertebrate skeletal development bones are mainly formed by endochondral ossification. Initially, mesenchymal cells condensate into chondrocytes which form cartilage anlagen, where a tightly controlled program of proliferation and differentiation is implemented. The hedgehog (Hh) signalling pathway is known to coordinate endochondral ossification for proper formation of the vertebral skeleton and disregulation causes severe skeletal disorders. Three hedgehog orthologs are known: Sonic hedgehog (Shh), Indian hedgehog (Ihh) and Desert hedgehog (Dhh). The ortholog Ihh is known to regulate chondrogenesis and limb formation in long bones, joints and digits, e.g. a point mutation in Ihh causes digit abnormalities. However, the role of Ihh and its subsequent signalling partner Patched1 (Ptch1) in spinal and chondral ossification remains obscure to date.

The analysis of gene function in spinal chondrogenesis is hampered by the lack of specific deleter mice. Various models are described in the art allowing analysis of the diseases, disorders or conditions. Said models are typically based on cell models, in particular, based on transgenic animal models. U.S. Pat. No. 6,884,775 describes the discovery that hedgehog proteins, and agents which effect activities thereof, can be used to control reformation and/or maintenance of cartilage and bone. An influence of the hedgehog single pathway on chondrogenesis and osteogenesis is known. For example, Yamaguchi et. al., Endocrine reviews, 2000, 21(4):393-411 describes said effects.

A commonly used model for B-cell specific gene deletion comprises the expression of the Cre recombinase under the control of the mb1 promoter which encodes the Ig-alpha signalling subunit of the B-cell antigen receptor.

The Hh signalling cascade has a well established control function in cell proliferation which is essential for differentiation and patterning in different tissue types including brain, skin and cartilage. Constitutive Hh pathway activity causes developmental abnormalities and tumorgenesis. Recent studies identified Ihh/Ptch1 signalling as a fine balanced process in limb formation and patterning.

Ptch1 (Patched1) represents a Hh receptor. The gene functions as a tumor suppressor: In the absence of Hh the downstream signal cascade vie Smoothened (Smo) is blocked through an interaction with Ptch1. Upon binding of Hh to Ptch1, the inhibition of Smo is abolished and the signal cascade is activated.

Arthropathy is a disease of joints including arthritis and arthrosis. Arthropathy is used to describe the following conditions: Reactive arthropathy, enteropathic arthropathy, crystal arthropathy, diabetic arthropathy and neuropatic arthropathy. Chondropathy refers to a disease of the cartilage. Typical diseases, disorders or conditions involving the cartilage include arthritis or spinal diseases. For example, chondropathy or arthropathy includes diseases, disorders or conditions involving hyperproliferation of chondrocytes. As a result, spine malformation may occur. A typical example of spinal malfunction is spinal ankylosis. Ankylosis is a stiffness of a joint due to abnormal adhesion in rigidy of the bones of the joint. A typical example is the spinal ankylosis or ankylosing spondolytis (AS) such as Bechterew disease or Bechterew syndrome. AS mainly affects joints in the spine and the sacroylium in the pelvis and cause eventual fusion of the spine.

### SUMMARY OF THE PRESENT INVENTION

In a first aspect, the present invention relates to a cell model containing chondrocytes whereby said chondrocytes contain a first heterologous nucleic acid sequence operably linked with a mb1 promoter. That is, it was recognised by the present inventors that mb1 known so far as a gene active in B-cells, is active additionally in chondrocytes. It has not been described before that mb1 promoter shows activity in chondrocytes. Thus, the mb1 promoter can be used to express heterologous nucleic acid sequences in chondrocytes.

In another aspect, the present invention relates to a transgenic animal model, in particular, a rodent transgenic animal model, like a transgenic mouse model. This animal model is characterised in containing chondrocytes whereby said chondrocytes contain a first heterologous nucleic acid sequence operably linked with a mb1 promoter. Preferably, said cell model contains a second heterologous nucleic acid sequence encoding a peptide of interest.

Further, a cell model is provided whose genome comprises a) a first heterologous nucleic acid sequence encoding a recombinase and/or a restriction enzyme operably linked to chondrocyte specific promoter, and b) a second heterologous nucleic acid sequence encoding a target of interest, like a peptide of interest, whereby the second nucleic acid sequence further comprises recombination sequences or restriction sites for the enzyme encoded by the first heterologous nucleic acid sequence.

In another embodiment of the present invention, a method of screening for an agent reducing or preventing one or more symptoms of arthropathy and/or chondropathy, in particular, spinal malfunction due to hyperproliferation of chondrocytes is provided. Said method according to the present invention comprises the use of a cell model according to the present invention, administering to said model an agent to be tested and determining whether the agent reduces or prevents one or more symptoms of arthropathy and/or chondropathy, in particular, of spinal malfunction due to hyperproliferation of chondrocytes.

Further, a method of testing the efficacy and efficiency of an agent suspected to allow treatment of diseases, disorders, conditions of arthropathy and/or chondropathy, in particular, involving spinal malfunction, like arthropathy of the spine is provided. Said method according to the present invention involves the steps of using a cell model, in particular, a transgenic animal, according to the present invention, administering to said cell model an agent suspected to be useful in the treatment of diseases, disorders or conditions of arthropathy and/or chondropathy, in particular, spinal malfunction, in particular, arthropathy of the spine, and determining the efficacy and/or efficiency of whether said agent is useful in the treatment of diseases, disorders or conditions of arthropathy and/or chondropathy, in particular, involving spinal malfunction, in particular, arthropathy of the spine.

### BRIEF DESCRIPTION OF THE DRAWINGS

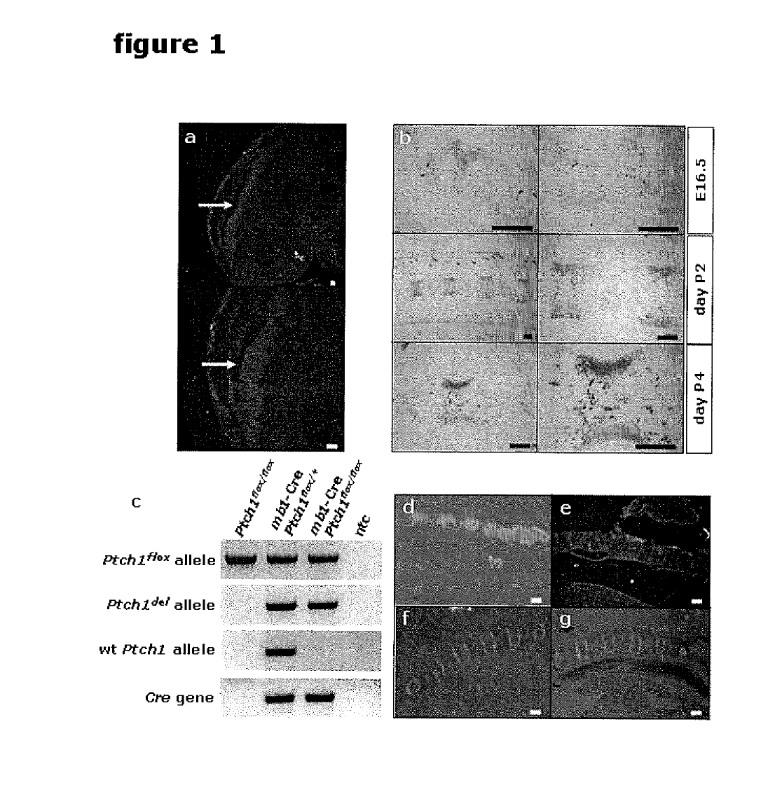

In FIG. 1 efficient mb1 promoter mediated Cre recombination of the floxed Ptch1 locus in the chondrocytes is shown.

1a) In situ hybridization on E12.5 sections of wild type mice with antisense riboprobes for mb1 (scale bar, 100 μm).

1b) LacZ staining of spinal sections of mb1-Cre; R26R+/− mice (scale bar 50 μm). Mice at embryo stage 16.5 dpc (E16.5, upper panels), two (day P2, middle panels) and four (day P4, lower panels) days after birth.

1c) Genotyping of DNA derived from spinal chondrocytes isolated from mice three days after birth. Ptch^{flox} allele (1735 bp), Ptch^{del} allele (950 bp), wt Ptch allele (1401 bp), Cre gene (600 bp), ntc indicates no template control. 1 d)-g) In situ hybridization of E16.5 spinal sections of mb1-Cre Ptch^{flox/flox} mice with antisense riboprobes for Cre (d), Gli1 (e), Ihh (f) and ColX (g) (scale bars, 100 μm).

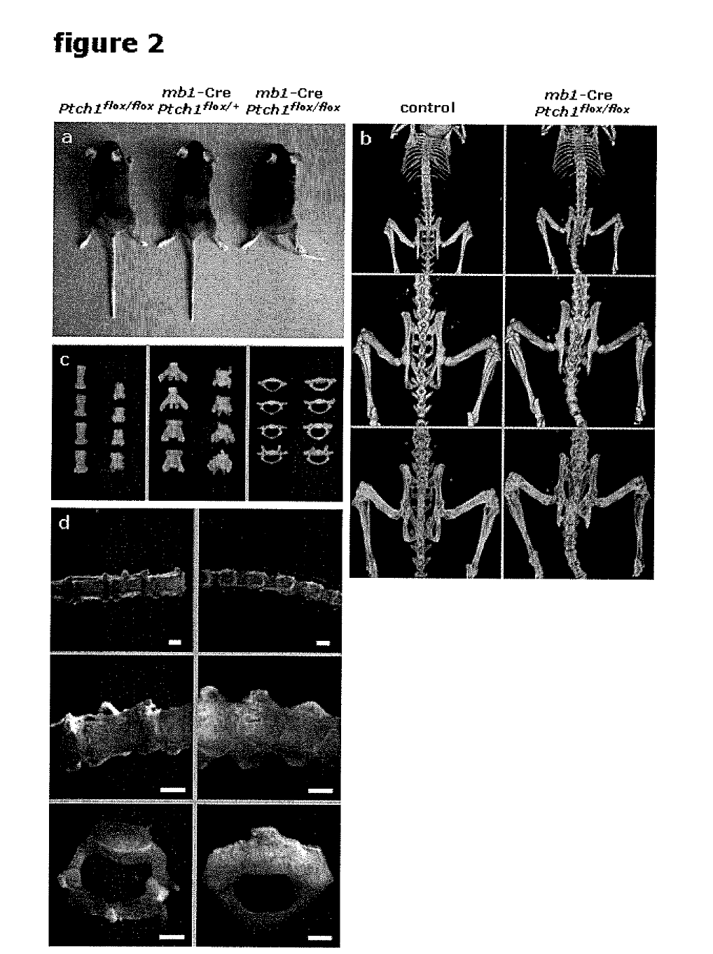

In FIG. 2 the results of deletion of Ptch1 in chondrocytes is shown. The severe spinal phenotype is demonstrated.

2a) Macroscopic image of littermates eight weeks after birth. Genotypes as indicated.

2b) Volume rendering of in vivo aquired fpVCT data sets (resolution 150 μm) depicting skeletal morphology of control (left panels) and mutant mice (right panels) six weeks after birth. Lower panels show volume rendering representing bone density of the 3D data set in the middle panel. Yellow areas indicate higher bone density in comparison to areas in grey.

2c) Macroscopic images of dissected vertebral bodies of tail (left), lumbar/thoracic spine (middle) and cervical spine (right) from mice twelve weeks after birth. In each separate picture vertebral bodies left were dissected from control and right from mutant mice.

2d) Alcian blue/alizarin red stained spine dissected from control (left panels) and mutant mice (right panels) four weeks after birth (scale bar, 1000 μm).

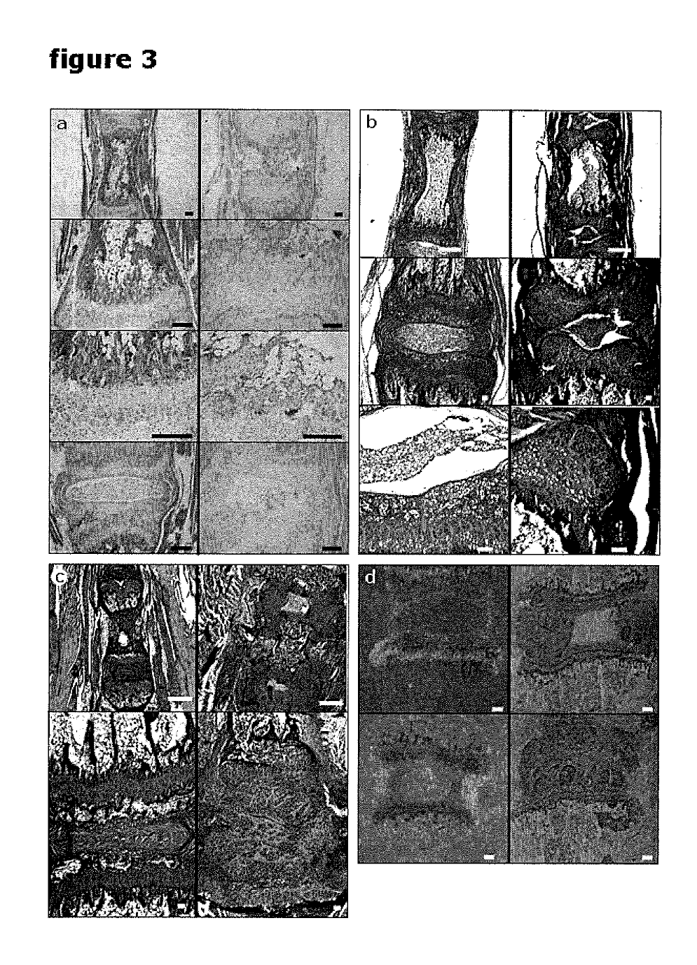

In FIG. 3 the effect of Ptch1 deletion in spinal chondrocytes is demonstrated. As shown, Ptch1 deletion in spinal chondrocytes causes hyperproliferation.

3a) Hematoxylin- and Eosin-stained tail sections of control (left panels) and mutant mice (right panels) four weeks after birth (scale bar, 50 μM).

3b) Safranin-Weigert staining (cartilage red, bone blue) of tail sections of control (left panels) and mutant mice (right panels) four weeks after birth (scale bar upper panels, 1000 μm, middle and lower panels, 100 μm).

3c) Safranin-Weigert staining of lumbar spine sections of control (left panels) and mutant mice (right panels) four weeks after birth (scale bar upper panels, 1000 μm, lower panels, 100 μm).

3d) In situ hybridization on tail, lumbar and thoracic spine sections of control (left panels) and mutant mice (right panels) four weeks after birth with antisense riboprobes for CoIIIa (scale bar, 100 μm).

### DETAILED DESCRIPTION OF THE PRESENT INVENTION

The present invention relates to new cell models wherein at least chondrocytes are genetically modified. That is, in a first aspect, a cell model containing chondrocytes whereby said chondrocytes contain a first heterologous nucleic acid sequence operably linked with a mb1 promoter is provided. It was recognized for the first time that the mb1 promoter is active in chondrocytes. So far the mb1 promoter has been described as a B-cell specific promoter. The mb1 gene encodes the Ig-alpha subunit of the B-cell antigen receptor. It has been recognized that heterologous nucleic acid sequence under the control of the mb1 promoter is active in chondrocytes. For example, the mb1 promoter comprises the sequence of any one of Seq. ID. No. 1, 2, 3, 4, or 5. Typically, the mb1 promoter is derived from the same species as the cell model is stemming from. For example, in case of a mouse model, the mb1 promoter of the mice is used or in case of a cell model based on human cells, the human mb1 promoter is used.

In this connection, the term “heterologous nucleic acid sequence” or “heterologous polypeptide” identifies a nucleic acid sequence or polypeptide put into a cell that does not normally contain or express said nucleic acid sequence or polypeptide, or the nucleic acid sequence or the polypeptide have been genetically manipulated by genetic engineering, e.g. by introducing restriction sites or recombination sites. Thus, the term “heterologous” refers to the fact that nucleic acid sequence or polypeptide is initially obtained from or derived from or contains elements from a different cell type or even a different species from the recipient cell type.

The term “cell model” as used herein refers to a genetically engineered cell model. Said cell model includes genetically engineered cells and cell lines, in particular, genetically engineered chondrocytic cells or cell lines or precursor or progenitor cells thereof. In another aspect, the cell model is preferably a transgenic animal model. Particularly preferred, said transgenic animal model is a rodent transgenic animal model, particularly preferred, a transgenic mouse model. Said transgenic mouse model is characterized in having chondrocytes whereby said chondrocytes contain a first heterologous nucleic acid sequence operably linked with a mb1 promoter.

In a preferred embodiment, the first heterologous nucleic acid sequence present in the cell model according to the present invention is transcribed to a coding or non-coding RNA, in particular, a mRNA, tRNA, miRNA or siRNA. In another aspect, the heterologous nucleic acid sequence encodes a polypeptide.

In this connection, the term “polypeptide” as used herein is intended to encompass a singular “polypeptide” as well as plural “polypeptides” and comprises any chain or chains of two or more amino acids. Thus, as used herein, said term includes oligopeptides of two to fifty amino acids length as well as polypeptides having at least 51 amino acids. The term further includes polypeptides which have undergone posttranslational modifications, for example, glycosylation, acetylation, phosphorylation, amidation, derivatisation by known protecting/blocking groups, protolytic cleavage or modification by non-naturally occurring amino acids. Furthermore, the term nucleic acid or nucleic acid fragment refers to any one or more nucleic acid segments, e.g. DNA or RNA fragment present in a polynucleotide. The term “polynucleotide” or “nucleic acid sequence” is intended to encompass a singular nuclear acid or nucleic acid fragment as well as plural nucleic acids or nucleic acid fragments. In certain embodiments, the polynucleotide, nucleic acid sequence, nucleic acid or nucleic acid fragment is a DNA.

In a preferred embodiment, the first heterologous nucleic acid encodes a polypeptide, in particular an enzyme. Particularly preferred, said enzyme is a recombinase, like the Cre recombinase or the FLP recombinase. Alternatively, said enzyme is a restriction enzyme.

In another preferred embodiment, the cell model according to the present invention wherein the chondrocytes contain a first heterologous nucleic acid sequence operably linked with a mb1 promoter contains in addition a second heterologous nucleic acid sequence encoding a target of interest, like a peptide of interest (POI). Said second heterologous nucleic acid sequence which is different from the first heterologous nucleic acid sequence comprises furthermore at least one sequence allowing site specific recombination of the sequence encoding the target of interest, like a POI, in particular, comprising at least one loxP site or FLP recognition target.

Said one sequence allowing site specific recombination of the sequence encoding a target of interest, e.g. a POI, like the loxP site or FLP recognition target, are located 5′ and/or 3′ of the second heterologous nucleic acid sequence encoding the target of interest, e.g. the POI. Preferably, at least 5′ of the second heterologous nucleic acid sequence, a first loxP sequence or FLP recognition target sequence is located. Particularly preferred, said sequence allowing site specific recombination of the sequence encoding the target of interest, e.g. the POI, is located on both flanking sites, i.e. 5′ upstream and 3′ downstream of the second heterologous nucleic acid sequence.

The second heterologous nucleic acid sequence is preferably at least a fragment of one of the sequences Ptch1, VDR, PTEN, SOX9, SMO, GLI1, GLI2, GLI3, BMP2, BMP7, RUNX2, PTH, PTHRP, PGF2, GDF5, EXT1, WNT5A, TNFRSF11A, TNFFSF11. In particular, the sequence is at least one of Ptch1, VDR, PTEN, SOX9, SMO, GLI1, GLI2, GLI3, BMP2, BMP7, or RUNX2, like Pcht1. Further data of said preferred sequences are given in table 1. It is particularly preferred that the second heterologous nucleic acid is at least a fragment of the Ptch1 nucleic acid sequence, e.g the nucleic acid sequence of Seq. ID No. 6 or 7 (Gene ID 5727 and Gene ID 19206, respectively), like at least one exon of Ptch1. The second heterologous nucleic acid may be present as part of the endogenous sequence, e.g. the Ptch1 sequence, of the cell. That is, the second heterologous nucleic acid sequence is flanked by at least one sequence allowing site specific recombination of the sequence of interest is integrated into the endogenous nucleic acid sequence, thus, allowing to disrupt the function of the nucleic acid sequence or the peptide encoded by said nucleic acid sequence. For example, the second heterologous nucleic acid sequence is exon 8 and/or 9 of the Ptch1 gene having loxP sites upstream and downstream and being integrated into the Ptch1 gene of the cell.

In another aspect, a cell model is provided whose genome comprises
a) a first heterologous nucleic acid sequence encoding a recombinase and/or a restriction enzyme operably linked to a chondrocyte specific promoter, and
b) a second heterologous nucleic acid sequence encoding a target of interest, like a peptide of interest (POI), wherein the second nucleic acid sequence further comprises recombination sequences or restriction sites for the enzyme encoded by the first heterologous nucleic acid sequence.

Particularly preferred, said cell model is a transgenic animal model, like a rodent animal model, and in particular, a transgenic mouse model.

Particularly preferred, said cell model, like a transgenic animal model, is a cell model wherein the first heterologous nucleic acid sequence contains the mb1 promoter as chondrocyte specific promoter.

The first heterologous nucleic acid sequence is preferably a recombinase, in particular, a Cre recombinase or a FLP recombinase. Furthermore, the cell model according to the present invention, in particular the transgenic animal model according to the present invention is a cell model wherein the target of interest, e.g. the POI, encoded by the second heterologous nucleic acid sequence is the Ptch1 sequence.

That is, provided herein, is a cell model, in particular, a transgenic animal, like a transgenic rodent, whose genome comprises a first nucleic acid sequence encoding a fusion polypeptide, wherein the fusion polypeptide comprises a recombinase operably linked to a chondrocyte specific promoter, in particular, mb1 promoter, and a second nucleic acid sequence encoding Ptch1, wherein the second nucleic acid sequence comprises one or more loxP sequences or one or more FLP recognition targets.

This cell model, in particular in form of a transgenic animal model represents a suitable model for arthropathy and/or chondropathy. In particular, said cell model, for example in form of a transgenic animal model, is a suitable model for spinal arthropathy, spinal diseases, disorders or conditions involving hyperproliferation of chondrocytes and malformation of the spine. Said diseases, tumors, disorders or conditions include rheumatoid arthritis, scoliosis, spondylitis ankylosans and other syndromes of the spine. Said cell model according to the present invention, in particular, in form of a transgenic animal model, is particularly useful as a model for spinal ankylosans.

In particular, the analysis of gene function in the spinal chondrogenesis is difficult by the lack of specific cell models, in particular, specific animal models. The present inventors aims to provide a new cell model, in particular, an animal cell model allowing to analyse and assess diseases, disorders and conditions involving hyperproliferation of chondrocytes and malformation of the spine. That is, an animal model for arthropathy and/or chondropathy is provided. Since it has been discovered that the mb1 promoter is active in chondrocytes, in particular, in spinal chondrocytes, mb1 Cre mice represents a valuable tool to specifically inactivate genes in respective chondrocytes, in particular, in spinal chondrocytes. Furthermore, these mb1 Cre mice have been crossed with Ptch1^{flox/flox} mice. It is shown herein that homozygous progeny animals shows Ptch1 conditionally inactivated in chondrocytes, in particular, in spinal chondrocytes. That is, deletion of Ptch1 via Cre activity leads to constituvely active Hh signalling.

It is known that constitutive Hh pathway activity causes developmental abnormalities and tumor genesis. In addition, it has been demonstrated recently that Ihh/Ptch1 signalling represents a fine balanced process in limb formation and patterning. By providing the animal model according to the present invention wherein the Ptch1 gene is inactivated due to the Cre activity and, thus, said animal model represents a model for the assessment of arthropathy and/or chondropathy, in particular, spinal arthropathy involving hyperproliferation of chondrocytes in malfunction of the spine. In particular, this animal model is particularly useful for studying spinal ankylosans. Further, it is possible to study the gene function in endochondral ossification, in particular, in spinal endochondral ossification.

Hence, the present invention relates in another aspect to a method of screening for an agent reducing or preventing one or more symptoms of arthropathy and/or chondropathy, in particular, spinal malformation due to hyperproliferation of chondrocytes comprising steps of
(a) providing a cell model according to the present invention;
(b) administering to the cell model an agent to be tested; and
(c) determining whether the agent reduces or prevents one or more symptoms of arthropathy and/or chondropathy, in particular, of spinal malformation due to hyperproliferation of chondrocytes.

In another aspect, the present invention relates to a method of testing the efficacy and/or efficiency of an agent suspected to allow treatment of diseases, disorders or conditions of arthropathy and/or chondropathy, in particular, involving spinal malformation, like arthropathy of the spine, comprising the step of
(a) providing a cell model according to the present invention;
(b) administering to said cell model an agent suspected to be useful in the treatment of diseases, disorders or conditions of arthropathy and/or chondropathy, in particular, involving spinal malformation, in particular, arthropathy of the spine, and
(c) determining the efficacy and/or efficiency of whether said agent is useful in the treatment of diseases, disorders or conditions of arthropathy and/or chondropathy, in particular, involving spinal malformation, in particular, arthropathy of the spine.

In another aspect, a progeny animal resulting from a cross between two transgenic animals is provided. The first transgenic animal's genome comprises the first heterologous nucleic acid sequence operably linked with a mb1 promoter. Preferably, said first transgenic animal's genome comprises the first heterologous nucleic acid sequence encoding the Cre recombinase or the FLP recombinase operably linked with a mb1 promoter. The second transgenic animal's genome comprises a second heterologous nucleic acid sequence encoding a target of interest, e.g. a POI. Said second heterologous nucleic acid sequence is different from the first heterologous nucleic acid sequence. In addition, said second heterologous nucleic acid sequence comprises at least one sequence allowing site-specific recombination of the sequence encoding the target of interest, e.g. the POI, in particular, comprising at least one loxP site or FLP recognition target. In another embodiment, the promoter operably linked with the first heterologous nucleic acid sequence is a different chondrocyte specific promoter and/or the second heterologous nucleic acid sequence comprising at least one sequence allowing site specific recombination of the sequence encoding the target of interest, e.g. the POI comprising at least one loxP site or FLP recognition site is Ptch1. That is, the gene encoding Ptch1 may be genetically modified in a way that a fragment thereof, e.g. one or more exons are flanked by at least one loxP site, either upstream and/or downstream thereof. The modified exon(s) is/are integrated into the Ptch1 gene and, after recombination, the activity of Ptch1 is disrupted.

Furthermore, provided herein are isolated cells of progeny animals resulting from a cross between the first and second transgenic animal. The isolated cell is in particular a chondrocyte.

The chondrocyte specific promoter, in particular, the mb1 promoter may be a constitutive promoter or an inducible promoter. For example, the promoter may be a tamoxifen inducible promoter, thus, inducing expression of the first heterologous nucleic acid sequence. In case the first heterologous nucleic acid sequence is a recombinase or restriction enzyme, said enzyme is present only after induction. Thus, it is possible to induce recombination at various time points.

Of course, the promoter and/or enhancer of the promoter and/or the heterologous nucleic acid sequence may be inducible by other chemical or physical entities. A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Alternatively, the promoter and/or enhancer region can act as a conceptive promoter and/or enhancer to maximise the expression of the region of the transcription unit to be transcript.

In this connection, the skilled person is well aware of other elements necessary to allow transcription and, eventually, translation of the first and second heterologous nucleic acid sequence.

Further, as used herein, the terms “treatment”, “treat” or “treating” refer to a method of reducing the effects of a disease or condition or symptoms of the disease or condition. Thus, in the disclosed method, treatment can refer to a reduction in the severity of an established disease or condition or symptom of the disease or condition. Said grade of reduction may be at least 10%, for example 20%, 30%, 50%, 80%, 90% or 100% reduction compared to native or control levels. It is understood that treatment does not necessarily refer to a cure or complete ablation of the disease, condition, or symptoms of the disease or condition.

As used herein, the terms “prevent”, “preventing” and “prevention” of a disease or disorder or condition refer to an action, for example administration of therapeutic agent, that occurs before or at about the same time as subject begins to show one or more symptoms of the disease or disorder, which inhibits or delays on said or exacerbation of one or more symptoms of the disease or disorder. As used herein, reference is to decreasing, reducing or inhibiting include a change of e.g. 10%, like 20%, 30%, 50%, 80%, 90% or greater as compared to a control level.

The cell model, in particular, the cell model in form of a transgenic animal model allows to test the efficacy and efficiency of agent known or suspected to allow treatment of chondrocyte malfunction in arthropathy and/or chondropathy, in particular, spinal malformation. In addition, the model allows screening for an agent reducing or preventing one or more symptoms of arthropathy and/or chondropathy, in particular, for the diseases, disorders or condition mentioned above.

The following examples are included for purposes of illustration only and are not intended to limit the scope of the present invention which is defined by the appended claims.

### EXAMPLES

### Material and Methods

### Generation and Genotyping of Mice

To generate mb1-Cre Ptch^{flox/flox} mice, mb1-Cre mice (Hobeika, E. et al., PNAS 103, 13789-13794 (2006)) were crossed with Ptch^{flox/flox} mice (Uhmann, A., et al., Blood 110, 1814-1823 (2007)). Genotyping of these mice and detection of Ptch^{del} alleles was performed as described previously (Hobeika, E. et al., PNAS 103, 13789-13794 (2006), Uhmann, A., et al., 2007, above). To obtain mb1-Cre; R²⁶R^{+/-} mice, mb1-Cre mice were crossed with the ROSA26 Cre reporter strain (R²⁶R) and genotyping on genomic tail DNA was performed as described previously (Soriano, P., Nature Genet. 21, 70-71 (1999)). All animal studies were undertaken with consideration of the necessary legal requirements.

### LacZ Staining

Frozen sections were briefly fixed in ice-cold PBS containing 0.2% glutaraldehyde for 10 min. After washing three times for 5 min at room temperature with LacZ buffer (PBS containing 100 mM MgCl₂, 1% sodium desoxycholate, 10% NP40), sections were incubated with lacZ staining solution (LacZ buffer containing 20 mg/ml 5-bromo-4-chloro-3-indoxyl-β-D-galactopyranoside (X-gal), 250 mM potassium ferrocyanide, 250 mM potassium ferricyanide) over night at 30° C. Next day slides were washed three times in PBS and mounted onto coverslips with Glycergel Mounting Medium (Dako, Denmark).

### Preparation and Genotyping of Spinal Chondrocytes

Tail and lumbar spine of mice three days after birth were rid of skin and muscles and incubated with pronase and collagenase D (Roche) as described previously (Mak, K. K., et. al., Development 135, 1947-1956 (2008).). Isolated primary chondrocytes were incubated in buffer (1 mM Tris/HCl, 5 mM KCl, 0.45% NP40, 0.45% Tween) with 10 mg/ml proteinase K (Promega) at 56° C. overnight. Next day proteinase K was inactivated at 95° C. for 10 min. Genotyping on genomic DNA was performed as described previously, (Uhmann, A., et al., 2007, above).

Histological analysis and In Situ Hybridization

For skeletal preparations, mice were euthanized by CO₂ inhalation. Skin and soft tissues were removed, and skeletons were fixed in 95% ethanol and stained with alcian blue and alizarin red S as described previously (McLeod, M. J., Teratology 22, 299-301 (1980)). Mice were dissected in PBS (Invitrogen), embryos were fixed overnight in 4% paraformaldehyde (Roth) at 4° C., dehydrated with increasing ethanol concentrations and embedded in paraffin wax (Roth). Adult tissues were fixed decalcified in 25% EDTA (pH 7.4) at 37° C. prior to embedding. Serial sections of 5 μm were stained or used for in situ hybridization. Safranin-Weigert staining was performed with a series of Weigert's hematoxylin (Roth), 0.1% fast green (Sigma) and 0.1% safranin O (Sigma) according to the staining procedure (Kahveci, Z., et al., Biotech Histochem 75, 264-8 (2000)). For radioactive in situ hybridization, antisense riboprobes were labeled with [P³³]-UTP (Hartman Analytic). Hybridization was performed in 50% formamide (Roth) at 70° C. as described previously (Mining, E., et al., Dev Cell 3, 439-49 (2002)). Developed slides were counterstained with 0.2% toluidine blue O (Sigma) in 1% sodium borate (Roth). References for in situ hybridization probes are as follows: Collagen Type 2 (Kohno, K., et al., J Biol Chem 259, 13668-73 (1984)), Collagen Type X (Jacenko, O., et al., Prog Clin Biol Res 383B, 427-36 (1993)), Gli1 (Hui, C. C. & Joyner, A. L. Nat Genet. 3, 241-6 (1993)), Ihh (Bitgood, M. J. & McMahon, A. P., Dev Biol 172, 126-38 (1995)). For the Cre-recombinase a 1031 bp fragment of the coding region of the P1 Cre Rekombinase was cloned in the pCRII-Topo vector. A 845 bp probe for mb1 was PCR amplified using the primer pair 5′-taccaagaaccgcatcatca-3′, Seq. ID. No. 8,5′-aggagggtgaggccctataa-3′, Seq. ID No. 9, and cloned in the pCRII-Topo vector. Pictures were taken with the Spot 14.2 or 23.0 camera (Diagnostik Instruments, Puchheim, Germany) using the Metamorph imaging software (Visitron Imaging Systems, Puchheim, Germany). Hybridization signals were visualized in darkfield (Intralux 5000-1, Vopi, Switzerland) and the expression domains were determined using Spot analysis software.

### Imaging and Statistical Analysis

Pictures were taken with the Spot 14.2 or 23.0 camera (Diagnostik Instruments, Puchheim, Germany) using the Metamorph imaging software (Visitron Imaging Systems, Puchheim, Germany). Hybridization signals were visualized in darkfield (Intralux 5000-1, Vopi, Switzerland) and the expression domains were determined using Spot analysis software.

Monitoring of Bone Morphology by Flat-Panel Volume Computed Tomography (fpVCT)

Monitoring was performed by noninvasive VCT using a laboratory animal fpVCT from GE Global Research as described previously (Missbach-Guentner, J., et al., Neoplasia 9, 755-765 (2007)).

Isolation of Vertebral Bodies from Mice Spine

Spines from CO₂-euthanized mice were dissected and incubated in buffer (1 mM Tris/HCl, 5 mM KCl, 0.45% NP40, 0.45% Tween) with 10 mg/ml proteinase K (Promega) at 56° C. overnight without shaking. Next day vertebral bodies were washed in dH₂O and ethanol to remove remaining tissue.

### Hematoxylin and Eosin Staining

Tails of mice four weeks after birth were fixed in 10% formalin for 24 h, decalcificated in EDTA, pH 7.41, for 48 h, and embedded in paraffin. For hematoxylin and eosin staining paraffin blocks were cutted in 5 μm sections, dewaxed and rehydrated.

Herein for the first time the present inventors identified mb1 gene locus activity in spinal chondrocytes (FIG. 1a). To determine Cre recombination in mb1-Cre mice (Hobeika of al., 2006, above), the present inventors assayed for Cre activity by mating mb1-Cre mice with ROSA26 reporter (R26R) mice (Soriano, 1999, above). Indeed, spinal chondrocytes express lacZ confirmed by β-gal activity due to Cre-mediated recombination (FIG. 1b). Hence the mb1-Cre mice is a valid tool to specifically inactivate genes in spinal chondrocytes.

To study the Ihh/Ptch1 signalling pathway in spinal chondrogenesis in vivo the present inventors crossed Ptch1^{flox/flox} mice (Uhmann, 2007, above) with mb1-Cre mice. In homozygous (mb1-Cre Ptch1^{flox/flox}) mice Ptch1 is conditionally inactivated in spinal chondrocytes (FIG. 1c). Deletion of Ptch1 via Cre activity (FIG. 1d) leads to constitutively active Hh signalling as determined by Glioma-associated oncogene family zinc finger 1 (Gli1) activity assayed surround the developing intervertebral dics for homozygous mice at E18.5 (FIG. 1e). At this embryonic stage spinal chondrocytes in the center of the cartilage anlagen synthesize Ihh and type X collagen (CoIX) indicating chondrocyte hypertrophy (FIGS. 1f and 1g).

All heterozygous and homozygous mice were born at the expected Mendelian ratio and appeared initially phenotypically normal within the first days after birth. Two to three weeks after birth mb1-Cre Ptch1^{flox/flox} mice showed an obvious aberrant tail development which is highly visible in the adult (FIG. 2a). All Ptch1^{flox/flox} and mb1-Cre Ptch1^{flox/+} remained phenotypically normal up to an age of 500 days and served as controls. In contrast to these animals mb1-Cre Ptch1^{flox/flox} mice showed the tail anomaly with complete penetrance. To exclude that B cells are involved in these phenotypical changes the present inventors generated mice which habor a Cre insertion in both mb1 alleles of their genome. As previously shown for mice with inactivated mb1 alleles the B cell development was completely blocked in the bone marrow (Pelanda, R., et. al., J Immunol 169, 865-872 (2002)). Despite the complete lack of peripheral B cells the mice developed spinal malformation in manner similar to mb1-Cre Ptch1^{flox/flox} mice. Hence, the phenotypical changes were caused by deletion of Ptch1 in spinal chondrocytes.

To analyze the skeletal morphology in more detail and monitor the disease progression in vivo the present inventors performed flat panel-based volume computed tomography (fpVCT). This method allows rapid noninvasive high-resolution 3-D imaging in the adult living organism (Missbach-Guentner, et al., 2007, above). An observation time from 6 weeks after birth up to 6 month revealed a severe spine phenotype with joined vertebral bodies which results in an pronounced spinal ankylosis (FIG. 2b). False color display indicates an enhanced relative spinal bone density in the mutant mice (FIG. 2b, lower left) compared to controls (FIG. 2b, lower right). Additional preparation of the vertebral bodies of tail and lumbar/thoracic spine of adult animals revealed a distinct modified spinal bone morphology whereas the cervical vertebral bodies show minor changes (FIG. 2c). Aldan blue/alizarin red stained spinal preparations prove the existence of joined vertebral bodies as implied by the fpVCT in vivo imaging (FIGS. 2b and 2d). In mutant mice the vertebral bodies of the spine were tightly connected with each other (FIG. 2b, upper and middle right panels) in contrast to controls (FIG. 2b, upper and middle left panels). Furthermore, the vertebral bodies of mutant mice appeared spherically enhanced and worse malformed which might lead to damaged intervertebral discs. Collectively these data propose a defective endochondral ossification which leads to spinal ankylosis. Histological Hematoxylin- and Eosin-stained sections discovered a massive hyperproliferation in the endochondral ossification of the spine which leads to pathological developed and connected vertebral bodies and deformed intervertebral discs (FIG. 3a). The present inventors analyzed Safranin-Weigert stainings to discriminate between bone and cartilage. As shown in FIGS. 3b and 3c the spinal ankylosis appears to be caused by hyperproliferation in the chondrocyte cell population. The defect in the spinal endochondral ossification is driven by an unlimited chondrogenesis. This is supported by CoIIIa expression in chondrocytes (FIG. 3d). Under physiological conditions proliferative chondrocytes exit the cell cycle and differentiate into hypertrophic chondrocytes and terminate endochondral ossification. Ptch-deficiency in spinal chondrocytes results in hyperproliferative chondrogenesis and causes spinal ankylosis in the living organism.

Thus, these mice according to the present invention are a model for testing drug toxicity and efficacy to prevent human spinal malformations. Furthermore, the identification of Cre recombinase activity in chondrocytes provide the opportunity to specifically study gene function in spinal endochondral ossification.

| | | Gene ID: | | Gene ID: | |
|---|---|---|---|---|---|
| Gene | Description | Mus musculus | URL | Homo sapiens | URL |
| VDR | vitamin D receptor | Gene ID: | http://www.ncbi. | Gene ID: | http://www.ncbi. |
| | | 22337 | nlm.nih.gov/gene/ | 7421 | nlm.nih.gov/gene/ |
| | | | 22337 | | 7421 |
| PTEN | phosphatase and | Gene ID: | http://www.ncbi. | Gene ID: | http://www.ncbi. |
| | tensin homolog | 19211 | nlm.nih.gov/gene/ | 5728 | nlm.nih.gov/gene/ |
| | | | 19211 | | 5728 |
| SOX9 | SRY-box | Gene ID: | http://www.ncbi. | Gene ID: | http://www.ncbi. |
| | containing gene 9 | 20682 | nlm.nih.gov/gene/ | 6662 | nlm.nih.gov/gene/ |
| | | | 20682 | | 6662 |
| SMO | smoothened | Gene ID: | http://www.ncbi. | Gene ID: | http://www.ncbi. |
| | | 319757 | nlm.nih.gov/gene/ | 6608 | nlm.nih.gov/gene/ |
| | | | 319757 | | 6608 |
| GLI1 | GLI-Kruppel | Gene ID: | http://www.ncbi. | Gene ID: | http://www.ncbi. |
| | family member | 14632 | nlm.nih.gov/gene/ | 2735 | nlm.nih.gov/gene/ |
| | GLI1 | | 14632 | | 2735 |
| GLI2 | GLI-Kruppel | Gene ID: | http://www.ncbi. | Gene ID: | http://www.ncbi. |
| | family member | 14633 | nlm.nih.gov/gene/ | 2736 | nlm.nih.gov/gene/ |
| | GLI2 | | 14633 | | 2736 |
| GLI3 | GLI-Kruppel | Gene ID: | http://www.ncbi. | Gene ID: | http://www.ncbi. |
| | family member | 14634 | nlm.nih.gov/gene/ | 2737 | nlm.nih.gov/gene/ |
| | GLI3 | | 14634 | | 2737 |
| BMP2 | bone | Gene ID: | http://www.ncbi. | Gene ID: | http://www.ncbi. |
| | morphogenetic | 12156 | nlm.nih.gov/gene/ | 650 | nlm.nih.gov/gene/ |
| | protein 2 | | 12156 | | 650 |
| BMP7 | bone | Gene ID: | http://www.ncbi. | Gene ID: | http://www.ncbi. |
| | morphogenetic | 12162 | nlm.nih.gov/gene/ | 655 | nlm.nih.gov/gene/ |
| | protein 7 | | 12162 | | 655 |
| RUNX2 | runt related | Gene ID: | http://www.ncbi. | Gene ID: | http://www.ncbi. |
| | transcription | 12393 | nlm.nih.gov/gene/ | 860 | nlm.nih.gov/gene/ |
| | factor 2 | | 12393 | | 860 |
| PTH | parathyroid | Gene ID: | http://www.ncbi. | Gene ID: | http://www.ncbi. |
| | hormone | 19226 | nlm.nih.gov/gene/ | 5741 | nlm.nih.gov/gene/ |
| | | | 19226 | | 5741 |
| PTHRP | parathyroid | Gene ID: | http://www.ncbi. | Gene ID: | http://www.ncbi. |
| | hormone-like | 19227 | nlm.nih.gov/gene/ | 5744 | nlm.nih.gov/gene/ |
| | peptide | | 19227 | | 5744 |
| FGF2 | fibroblast growth | Gene ID: | http://www.ncbi. | Gene ID: | http://www.ncbi. |
| | factor 2 | 14173 | nlm.nih.gov/gene/ | 2247 | nlm.nih.gov/gene/ |
| | | | 14173 | | 2247 |
| GDF5 | growth | Gene ID: | http://www.ncbi. | Gene ID: | http://www.ncbi. |
| | differentiation | 14563 | nlm.nih.gov/gene/ | 8200 | nlm.nih.gov/gene/ |
| | factor 5 | | 14563 | | 8200 |
| EXT1 | Exostosin1 | Gene ID: | http://www.ncbi. | Gene ID: | http://www.ncbi. |
| | | 14042 | nlm.nih.gov/gene/ | 2131 | nlm.nih.gov/gene/ |
| | | | 14042 | | 2131 |
| WNT5A | wingless-related | Gene ID: | http://www.ncbi. | Gene ID: | http://www.ncbi. |
| | MMTV integration | 22418 | nlm.nih.gov/gene/ | 7474 | nlm.nih.gov/gene/ |
| | site 5A | | 22418 | | 7474 |
| TNFRSF11A | tumor necrosis | Gene ID: | http://www.ncbi. | Gene ID: | http://www.ncbi. |
| | factor receptor | 21934 | nlm.nih.gov/gene/ | 8792 | nlm.nih.gov/gene/ |
| | superfamily, | | 21934 | | 8792 |
| | member 11a (also | | | | |
| | known as RANK, | | | | |
| | Trance-R) | | | | |
| TNFSF11 | tumor necrosis | Gene ID: | http://www.ncbi. | Gene ID: | http://www.ncbi. |
| | factor (ligand) | 21943 | nlm.nih.gov/gene/ | 7421 | nlm.nih.gov/gene/ |
| | superfamily, | | 21943 | | 8600 |
| | member 11 (also | | | | |
| | known as RANKL, | | | | |
| | Trance, OPGL) | | | | |

1. A cell model containing chondrocytes whereby said chondrocytes contain a first heterologous nucleic acid sequence operably linked with a mb1 promoter.

2. The cell model according to claim 1 wherein the first heterologous nucleic acid sequence is transcribed to a coding or non-coding RNA, in particular, a mRNA, tRNA, miRNA, siRNA.

3. The cell model according to claim 1 wherein the heterologous nucleic acid sequence encodes a polypeptide, in particular, an enzyme.

4. The cell model according to claim 3 wherein said enzyme is a recombinase, in particular, the Cre recombinase or the FLP recombinase, or said enzyme is a restriction enzyme.

5. The cell model according to claim 1 wherein the chondrocytes contain a second heterologous nucleic acid sequence encoding a target of interest, like a peptide of interest (POI) whereby said second heterologous nucleic acid sequence which is different from the first heterologous nucleic acid sequence further comprises at least one sequence allowing site-specific recombination of the sequence encoding the POI, in particular, comprising at least one loxP site or FLP recognition target.

6. The cell model according to claim 5 wherein the POI encoded in the second heterologous nucleic acid sequence is at least one of Ptch1, VDR, PTEN, SOX9, SMO, GLI1, GLI2, GLI3, BMP2, BMP7, RUNX2, PTH, PTHRP, FGF2, GDF5, EXT1, WNT5A, TNFRSF11A, TNFFSF11, in particular, at least one of Ptch1, VDR, PTEN, SOX9, SMO, GLI1, GLI2, GLI3, BMP2, BMP7, or RUNX, like Ptch1.

7. The cell model according to claim 1 characterised in being a transgenic animal model, in particular, a rodent transgenic animal model, particularly preferred a transgenic mouse model.

8. A cell model, in particular, a transgenic animal model, whose genome comprises
a) a first heterologous nucleic acid sequence encoding a recombinase and/or a restriction enzyme operably linked to a chondrocyte specific promoter, and
b) a second heterologous nucleic acid sequence encoding a target of interest, like a peptide of interest (POI) wherein the second nucleic acid sequence further comprises recombination sequences or restriction sites for the enzyme encoded by the first heterologous nucleic acid sequence.

9. The cell model according to claim 8 wherein the first heterologous nucleic acid sequence contains the mb1 promoter as chondrocytes-specific promoter.

10. The cell model according to claim 8, wherein the first heterologous nucleic acid sequence encodes a recombinase, in particular, a Cre recombinase or a FLP recombinase.

11. The cell model according to claim 8 wherein the heterologous nucleic acid sequence is at least one of Ptch1, VDR, PTEN, SOX9, SMO, GLI1, GLI2, GLI3, BMP2, BMP7, RUNX2, PTH, PTHRP, FGF2, GDF5, EXT1, WNT5A, TNFRSF11A, TNFFSF11, in particular, at least one of Ptch1, VDR, PTEN, SOX9, SMO, GLI1, GLI2, GLI3, BMP2, BMP7, or RUNX, preferably, the Ptch1 sequence.

12. The cell model according to claim 8 wherein the recombination sequence or restriction sites for the enzyme encoded by the first heterologous nucleic acid sequence are recombination sequences selected from one or more loxP sequences or one or more FLP recognition targets.

13. The cell model according to claim 8 being a transgenic animal model, in particular, a rodent transgenic animal model, like a mouse model.

14. The cell model according to claim 8 suitable as a model for arthropathy and/or chondropathy, in particular, spinal arthropathy, spinal diseases, disorders or conditions involving hyperproliferation of chondrocytes and malformation of the spine, like rheumatoid arthritis, scoliosis, spondylitis ankylosans and other syndromes of the spine.

15. The cell model according to claim 14 being a transgenic animal model for spinal ankylosis.

16. A method of screening for an agent reducing or preventing one or more symptoms of arthropathy and/or chondropathy, in particular, spinal malformation due to hyperproliferation of chondrocytes comprising steps of
(a) providing a cell model according to claim 8;
(b) administering to the cell model an agent to be tested; and
(c) determining whether the agent reduces or prevents one or more symptoms of arthropathy and/or chondropathy, in particular, of spinal malformation due to hyperproliferation of chondrocytes

17. A method of testing the efficacy and/or efficiency of an agent suspected to allow treatment of diseases, disorders or conditions of arthropathy and/or chondropathy, in particular, involving spinal malformation, like arthropathy of the spine, comprising the step of
(a) providing a cell model according to claim 8;
(b) administering to said cell model an agent suspected to be useful in the treatment of diseases, disorders or conditions of arthropathy and/or chondropathy, in particular, involving spinal malformation, in particular, arthropathy of the spine, and
(c) determining the efficacy and/or efficiency of whether said agent is useful in the treatment of diseases, disorders or conditions of arthropathy and/or chondropathy, in particular, involving spinal malformation, in particular, arthropathy of the spine.


SEQUENCE LISTING (7 sequences)

SEQ ID NO: 1 - DNA, 1000 bp, Homo sapiens
ccatagctggcctgtttttttaaaaaaagaattccagtaaagagctgatcatggttctcactccttgaataccaggaacaccatctcgtatcacataatgagacagggagacattctggtcctcatctcacagatgaaaaatgtcaagcttcgaaggatcaaagtgcccacctagtcacacgggtagtcagccacaggtcagcctgccttatttattcttcatgagtatttatagtgactaacatttactgggcgcctactgtgggccatttctgtgcatgtgacaacccctttaagtccttgtttctaatcccaagaagcaaggaaatggggtcagggaagggacaaggtttgcccaagtccaggcagggggagaggtcaagctcagaaccatcacctgcccatgacacatgcccaggactcaggttccctaggcttccttccaaaggctcagcagtgacgagccagcccttgaaccagcctcttcccccacccaagcagccacctctcaggggaattgtggccaccacaggtgcagggagcagtttctctccactcacagcctgaagcatacccggcaggggctgtccccaggcccaacaagcaaagggcccagtagcgagggccactggagcccatctccggggggctgggcaggaagtagggtggggtttggggtagggatctggtaccctgggactgctgcaactcaaactaaccaacccactgggagaagatgcctgggggtccaggagtcctccaagctctgcctgccaccatcttcctcctcttcctgctgtctgctgtctacctgggtatgtggccaaagggcaggaactggcgggaggtgggggaagctgtggaggctgcagagagggcacaggcagagggaagggggctcagggaaaggggaagaggaggcagaggataggggacccagggaagatgcctatagaaatcgtatctgtgccaagatgggccaaggtggggctggagggag

SEQ ID NO: 2 - DNA, 1000 bp, Homo sapiens
cagtaaagagctgatcatggttctcactccttgaataccaggaacaccatctcgtatcacataatgagacagggagacattctggtcctcatctcacagatgaaaaatgtcaagcttcgaaggatcaaagtgcccacctagtcacacgggtagtcagccacaggtcagcctgccttatttattcttcatgagtatttatagtgactaacatttactgggcgcctactgtgggccatttctgtgcatgtgacaacccctttaagtccttgtttctaatcccaagaagcaaggaaatggggtcagggaagggacaaggtttgcccaagtccaggcagggggagaggtcaagctcagaaccatcacctgcccatgacacatgcccaggactcaggttccctaggcttccttccaaaggctcagcagtgacgagccagcccttgaaccagcctcttcccccacccaagcagccacctctcaggggaattgtggccaccacaggtgcagggagcagtttctctccactcacagcctgaagcatacccggcaggggctgtccccaggcccaacaagcaaagggcccagtagcgagggccactggagcccatctccggggggctgggcaggaagtagggtggggtttggggtagggatctggtaccctgggactgctgcaactcaaactaaccaacccactgggagaagatgcctgggggtccaggagtcctccaagctctgcctgccaccatcttcctcctcttcctgctgtctgctgtctacctgggtatgtggccaaagggcaggaactggcgggaggtgggggaagctgtggaggctgcagagagggcacaggcagagggaagggggctcagggaaaggggaagaggaggcagaggataggggacccagggaagatgcctatagaaatcgtatctgtgccaagatgggccaaggtggggctggagggagcccagcgaaggagaaggggcgtccacagtctcac

SEQ ID NO: 3 - DNA, 1000 bp, Mus musculus
ggccgggcggtggtgacgcacgcctttaatcccagcacttgggagtcagaggcaggcagatttctgtgttcgaggccagcctggtctacagaatgagttccaggacagccagggctacacagagaaaccctgtctcgaaaaacaaaaacaaaacaaacaaacaaaagctccatcagttaaaaataatcaaacaaaggtggcaataaacaaaggactgttgtcacctgcatagtggatacaaggccaacccgtgctatatgaaaccctgtttcaaaaaaataaaagaaagagagagaaaaggtggtgggttagaaagaaagaggcagataggagtgataagttttgcccgatgttagtgagcgcagaagccaaagttgagagtagccgctgtggacctgcctgcagcaggtacccagaaccatggctcgccaggatcctttctcagggatcagtggtgatgaaccaggccttgaaccaccctctccccgaccccacgcactagagagagactcaagggaattgtggccagcccaggtgcagggcagttcctctccactcagagcccacacatatggcaaataaagggccaggagtaagggccactggagcccatctccggcacggctgaacaggaagtgaggcggagtttggagtggaatctggtacctcaaggctacggccacccaaactaaccgactcactgggagacgatgccagggggtctagaagccctcagagccctgcctctcctcctcttcttgtcatacgcctgtttgggtaatgtggccaaagggaaggtgcgggtggggcgggggtgttgcggctgacaggagaggtgcagagtggagtcagaaatggccaggcagggtgatgcataacgtggcatctcttaaagtaacgccagaactctaggaaactgaggaaggaggaacactgcatttgaggccagcctgggctacataatgagttctatctaggccaacctgggcaagttactgat

SEQ ID NO: 4 - DNA, 1000 bp, Mus musculus
aataaagcttcatcaggccgggcggtggtgacgcacgcctttaatcccagcacttgggagtcagaggcaggcagatttctgtgttcgaggccagcctggtctacagaatgagttccaggacagccagggctacacagagaaaccctgtctcgaaaaacaaaaacaaaacaaacaaacaaaagctccatcagttaaaaataatcaaacaaaggtggcaataaacaaaggactgttgtcacctgcatagtggatacaaggccaacccgtgctatatgaaaccctgtttcaaaaaaataaaagaaagagagagaaaaggtggtgggttagaaagaaagaggcagataggagtgataagttttgcccgatgttagtgagcgcagaagccaaagttgagagtagccgctgtggacctgcctgcagcaggtacccagaaccatggctcgccaggatcctttctcagggatcagtggtgatgaaccaggccttgaaccaccctctccccgaccccacgcactagagagagactcaagggaattgtggccagcccaggtgcagggcagttcctctccactcagagcccacacatatggcaaataaagggccaggagtaagggccactggagcccatctccggcacggctgaacaggaagtgaggcggagtttggagtggaatctggtacctcaaggctacggccacccaaactaaccgactcactgggagacgatgccagggggtctagaagccctcagagccctgcctctcctcctcttcttgtcatacgcctgtttgggtaatgtggccaaagggaaggtgcgggtggggcgggggtgttgcggctgacaggagaggtgcagagtggagtcagaaatggccaggcagggtgatgcataacgtggcatctcttaaagtaacgccagaactctaggaaactgaggaaggaggaacactgcatttgaggccagcctgggctacataatgagttctatctaggccaacct

SEQ ID NO: 5 - DNA, 1000 bp, Mus musculus
gccgggcggtggtgacgcacgcctttaatcccagcacttgggagtcagaggcaggcagatttctgtgttcgaggccagcctggtctacagaatgagttccaggacagccagggctacacagagaaaccctgtctcgaaaaacaaaaacaaaacaaacaaacaaaagctccatcagttaaaaataatcaaacaaaggtggcaataaacaaaggactgttgtcacctgcatagtggatacaaggccaacccgtgctatatgaaaccctgtttcaaaaaaataaaagaaagagagagaaaaggtggtgggttagaaagaaagaggcagataggagtgataagttttgcccgatgttagtgagcgcagaagccaaagttgagagtagccgctgtggacctgcctgcagcaggtacccagaaccatggctcgccaggatcctttctcagggatcagtggtgatgaaccaggccttgaaccaccctctccccgaccccacgcactagagagagactcaagggaattgtggccagcccaggtgcagggcagttcctctccactcagagcccacacatatggcaaataaagggccaggagtaagggccactggagcccatctccggcacggctgaacaggaagtgaggcggagtttggagtggaatctggtacctcaaggctacggccacccaaactaaccgactcactgggagacgatgccagggggtctagaagccctcagagccctgcctctcctcctcttcttgtcatacgcctgtttgggtaatgtggccaaagggaaggtgcgggtggggcgggggtgttgcggctgacaggagaggtgcagagtggagtcagaaatggccaggcagggtgatgcataacgtggcatctcttaaagtaacgccagaactctaggaaactgaggaaggaggaacactgcatttgaggccagcctgggctacataatgagttctatctaggccaacctgggcaagttactgatt

SEQ ID NO: 6 - DNA, 73984 bp, Homo sapiens
acccaggagggctgtgctgatagcacgttcctcgagtttactttgctttccttgagtttattgtaaaggggtaaagttttcggatccgtcacgtgaccctgacaggtcctgcctatggcgcggcagaccacccacgccgagggccatggaactgcttaatagaaacaggcttgtaattgtgagtccgcgctgcactccgccgaaagcctccggcggcccagcgcgccggggtttttacactttccgttccttttgtaaagacggaggaggaggagaagaagaagaagaaaacggaggagaagaaaaagacgacaggggagacaaagagacccgcagcgacaaggcaagggggagacgagggaagactgggagaagacggaggagcggaggacgaggaaaggggggccagggaaaaaaaaggaattgatgtgaaatccaagcccagcgtccgcgccatcggcacccgcgctccgagcaggggttgacggccggctatggtgagtgcagccagcgcggcggccgccgacgccacctcgcctcgcgcgccgtgctcctcgggcggcgcggggacacggggacgcgggacgccccgcgcggcggacggaggagcgagccccgagcgccgggcaggaggggcgggccgcgcgccggggccgtggagccgggtgggctgcgccgcctgggcgcgggagcgcgggtcccgcgcctccacgctcgctcgcacgcccgggcccccgcccccgaggccagccccgcgtggcggggagggggctggccgcggcgggccccggctgaggaacggctccggggggaattctcacggagggggtcgccggagctcccattttcacgcctgaaagggtcgccaccttggcttccgctcatcagcctggtggtggctactgactcgccagcggaggcagagctggtgatggaagggctggtgcggagagtgacagcgccgaggggagggggctcagagcgcgcgcgcggaagccgctgccgccagctcgcgggagcagccgcagccgcagggaagggcacgctcgcgccccgcagacccgctctcgagcccgcacgcacgcctaggggtgcgggacacccgggctagaagagacgcaccttacccacctcccctcccatgctgcgggaaaagggccagagaggcttttggagttctggaaggggcggtgggtaagagaggtggtgtgtgtgactcttatgcccaccgattcaggcgcttgggtggctgccacccttacagagatgggcgaccagcccagtgcttagagtgcagagtgcggcctcaggggagcccgggggcgggccggaccgattctcactttacaggcccgggcccagcgctgaaccccaagacccccagaaactgtgcctctgggtgacatgcgctgtgtctccacgtatttagtacgttcatgtttaaaatgatgctagcgtgtggatgagcacactctcagggaaactgatctgtttccatcacctaagaagaaccatatgggtgggcggctgtggaagacacccacagacctgttttcctgcacctgggagagaagaccgtcttcctccctttttatcctttcttcctccctggggaggagaaatacaaaagaactagacataaataataaagagccacaatgggtggctctaaatatttgataattggatcactgaggtcttgaactaccacaatagtcctttttcagcattctgtgtgtgtgcatttcttttaggtttttatttatttctgtgtttattggcgacagtagttcagcatgcaattaatcgcctccacaaagaaagacaatgctttatggtgtcatcctttgttattgcaacttaaaatttctgggtggtactccggataaattgttgcctcaggtgcatggagtgcccaagttgtaaaacaaggggctttcgattgtgagtttatgtgtgttccagttgtcacccagcttccttccacaaggctataaaatgcctttgagtccatgatctgtttcaattaaaatgttgaatcaaggtagacatcaataggccccgccattctcaatttggggtattagcttcatttcaaactcactgtgagtggtgtatctaatttctggttataaagtagtcataatctttgaagctatttgaacaaaacttagaaccatttggaattattttctgaaaagaaaaaggctgttttacaaaattatcttcaactttctacagatagataaactgatgaggttaatcaatgtggattaaaatatgtctcccgtttttatcctcattcctctctctctctcacacacacacacacacacacacacaccttttattttctccagcctctgccacagtgtataatgccctattgagacgtgtaggcgtgatgtgtaaaccggattagcagtatgaaagaaagacacacaataagctggaattgttttacttccctttcatttctcatgcctatatttctgataaatttgttgctccagcagtgggtcaaagggatcatgttgctcagcttctttttcatgcaggacccaatagcagagatcactgaaatgaatggtgtgcctcctagcaaacgcaaagcttgatagagatatattagacagggagaggcgaggagagggaaggatgcaaaacatgttcaaagtctgggtgttgtaggaatacatctctatccatgagaggaaagcaatgtgggtaagaatatatttggagagagagggatcaatgcaggggtcaatgctctgttttctttcaagttcatagtaggctctgaacattcagcagttggtgacatgcttagcaagagaataccaatagcttttggcaatacataaagtctaattcccctttcaagctagtatttagacctggagtaatcaatgtggagatttctgggaccttctcaggacactcccctctgccgctttccccacccccagctcactgtaagctcaaaaaatgccagtgctcccgggtttgtgctgtagacccctcatttactcctttgtggagttaggagcagcttcccattaaaggagatcaggggtaattagcagcattattttgacaagttggctagagctgttccctttcctccagcctggggagggggtaaataaggggaggaaacagaaggtggaagggagtaggggaggggagtcaagcccagtagttttaaattttgtagctgctgaatctccccccaccccgtccccgacttccctgaaaccttttagactcattttctaccatcaccttttaagaatatacaaaagctgccaatgagaaaatagtaaggcattcttagggagccaagaaaatgtggtggatggtggcacattggcttgagttgagtcgactcaactcatattcaaggtacctgcttggaactgctgaagccctgccttgaccatcactcacatcctgtatcctgggctccccatgcagtaaaagaatattgcatcaaatccccaattcctctctccatccgaccctctcaacaaaaatactgtaggacttaaaatcatgtttgcaattccttgtatttctggaagagtattaaactcagtagacaaaattataaatggccccagggtcaaattactggaatacattaacccctttattcatttatactctgcaaaacttgccaaattacattggattttaaagactcatttggtttgatctgagaaaccagtgaaaagttaaatctcactgtggtttttcataactccaccaagtctggcttccacaattgcttaccattggaacatcccaacacaaagggcagatgttttatttcattgtcatcatttggataatctgatgtcatttttgaaatgtgtgtggtgtggccatggatatttgtagtattggaacagagaagggaggagatgtgagggaatccggagagcttaaggcatctgctttgaaaactacacactccaatgagaagtcttggatcatgactgggagaccaaaatgacaatttatgcattgagatgaactttacttggaaaggggaaatggaacactcagctgttgcttcataaaggctggcaactcctgctggctgataaggccccaagttctcacttatttttatttggtaactgtcacaaggggtgccagaggcagaagggccatttaaagggactgtcttcccggaggacaccttagcaggtctggctgccccagtctgcatctcagatctgtggaatccttgtggttagttgaagttaacaaacccggtgtggcacagcagtcccctgcacagggcatgcatattaaagccgacaatgcttacaaatgtaaataagggcagattgattaggaacaacattatcgctgggaatgtcgcactcgcatgcatcagcaatgaggcaaattgggggcagcctcagccctgaccacccaagtcgagcaggcttttacactggctcctcttccctttcttaaaacgttacagaaacaaggccaattagatcctcaaagtaattcatctctaagtatgaaaaaagcagaccacaggggaagcggcgggtggggggtggggagattaagatagcgctcgttagagccgaggctccggagtgcgtcctggaacccacgggtagaatttacaggccgggaccattgtctgagatgctgaaaagttaacccatcaaacagcgggcgagggggctcagggcactgaaagctccggggatcaatgttcaaaacgtttaggaattaagtcaaactaagggatatgggcctgagtggaagaggctttgccactgaaatgacgtgcatgtggcagccgccggaaccagaggacatgcagccgcgtgcggagagtgaggcggatccattacgggcacacagcttaccatttccttccagaattccttcttccaaacaagctatactgaaatccctctagtaaaagattgagacgaaacactaatagttttgggccaaaacgtggtggagaagtttgaaaagacaaacatagcaacgtgtctttggctatatggttccttgccctccagcaagcttgaaatataaaaaggctaacatgcaaaactaggggtactacccccctcccccagtttttaaaaagcaatttccagaagcaaacacacaatggttcgacttgaagtgtcggcataaccattcagtaagaaaagctagaatagtaaacctcttttcctgttttgtgctatttatcaaacctaataaacatcaggatcgtactcaaagatgaaactggcaatgtgtggagccgtctgagctctttatcaactcagtgtcattttcctcgcatgctgcccgcctgctttccgagtttccgcagcccttccctgaggcactcacaaaatcacgtgctccttaagcggtctgagttttgaatttccgcgctggcccggtgctcaggtccccctccggatcccgcgaacgcccccaaacacctccaccccgtctccctctcttcctcgggacatcccgccctgaccccggcaccgcatacggtttgcgggttagggggtcgggggaagggcctccccctgaagccggggaggaggagaggctggagggaaggtggccaggctggtgggggaggggagattgagggagggagcggggcgagggaggaaggagaaggcgcaggccgaaggaaggaagcggggaggggaaaccgccggggaggaaggaagggggcggggagagccggcggacgggccccctgcgggagccgcgtggaccgccccggtggccggcgcgccccgccgcagcccccagcgcgcccgggcagccagcgcggggccgctgaatgggcagggactcgtgagcgcgaccattggccggtcgcggggaggcggcgcgggcattgggcagcgtaagggggccggtccgccttcaggaatgtactacggcgcgtccggcacgtccgtccgcgccggctgggcgcgcgcctcgcctgggctctcgggagaccgcgctggaggcccggagccggcccggcgcatccgcctccctacctactctgccccggccgcgcgggcgcggggagtccgggagccgggcgcgcgcgcgcaccgggcctcctggcgcggggtcacgtggccaccggagctccgccttctccctgctcctccccctttccctccatctgggtttctttctttcctttcctttctttcttctttttttttcaatgaacatacaccgtgcgcatcttaaagccacaccgccccatttcacgacttcactagacttctcgctactgccagtccctctgcagttcaaccccgtttaaaaaaaaaaataaagtgaagcaaatcaagggtttgcgggtcgggttcctaccgcagggaggaacgtaataacttggaaacgtaagaaaaacacattccactttccgaaaacaaaaatttttttaaataaaaagcctagttgtgtagacgtgtctggctccagaatccacctcaaagggtcgcgtgttttccccctccgcgggaagcgaatccccgggatgccttggtgacactggacgcggtgtcgggacgaatcctcgcgatccgcgcacagtttgttttcaatgaatccagcatcggggcatttataatctcctacgtcatactattattaaatataacatgcataccgagttttgtaaaaaaaaatactgactatataaacatctggttaataaaaaggggtgtacaaatgaacccaaaataatcaggaaaactttactctctcaaggaaacaattccgtttctctaagttgtaaaaaaagattctgttgtttctcttttacttatgtcacattcgagattcaaataaaaacgcctctgcctctacaagactgcttcggtgtctggattccacgtttatagaacactgactccttcaaaccaagactgtgttttttttttttttaagttggggggagaaagcggctggggggcagacggattttaagaaagatggcttttcccaacgtccttgctttcctctttaactttccattctgtttatcttaatgcgaggcgttgttccctgcaagagcctaagtcttccctttagttataaaagaaaagagaagaggaaaaaaaaatctggcacacactggcgcactatccagcacgctgtcattattggtgcaggcctgggaatgcgagccggacctcccagtatttgctgcagcttgaggccaaggtgcctctgaaaggcttttctttgaggaatttcagaaattgtttaagcgcttaaaaaaatattacatttccccccgtgtctaccgcgaggcaaatgttcgtgtgtgtgcgtgcgcgcgcgtgtgtgcgcgcaaaggcatcccacaaaaagaagacactgttgaaaagaaaaggaataagagagaaaggagggggagcattccttaatggaagtattgcatgcgagaaggttggcgctctcagaggctgccccgaagccgaggatgcacacactgggttgcctacctgggtggtctctctactttggtgagctgttgctaagcagctaataatagtcatgtttgctgagtaatttctgccctccgcgagccaatcgcgtcgcagaaacccaagccatctgacagccccgggggcgggagctccagcctttttctctttcgctcgctctctccccctccaccccctcccctcccctcctcctcttctttctccaaatggagagagttcttttttttcttcttccatctcatctattgaatcaaggagctgctgcggccgctgcccgctgcacacacacagagcggagtccccaggtcccgggagcgagagaggcgcgctgcacggggacagaatggtccagcgggttgccgaggagcacaagaaagcagagtccgggaccgagcagccaccgcgaacccagcagccagagcccgagcagcccgagcagcagctcctggagccgccaccgccagcagcagccaccgcgggagcagcggcagctgcggctgcccgggcccggcagcgctgagacccgccgggcaccctcggaccccgcggcggcggcggctctgcgcttgccctccgcggccgctcgggcgacccgggaggcgccgagagagccagcgcggccggcgggagcagcggggattcgctggctctttctgcagtgaaggggtcgcggcgcggggcggggggcgggtagggggagttggggaccgcaaggagtgccgcggaagcgcccgaaggacaggctcgctcggcgcgccggctctcgctcttccgcgaactggatgtgggcagcggcggccgcagagacctcgggacccccgcgcaatgtggcaatggaaggcgcagggtctgactccccggcagcggccgcggccgcagcggcagcagcgcccgccgtgtgagcagcagcagcggctggtctgtcaaccggagcccgagcccgagcagcctgcggccagcagcgtcctcgcaagccgagcgcccaggcgcgccaggagcccgcagcagcggcagcagcgcgccgggccgcccgggaagcctccgtccccgcggcggcggcggcggcggcggcaacatggcctcggctggtaacgccgccgagccccaggaccgcggcggcggcggcagcggctgtatcggtgccccgggacggccggctggaggcgggaggcgcagacggacgggggggctgcgccgtgctgccgcgccggaccgggactatctgcaccggcccagctactgcgacgccgccttcgctctggagcagatttccaaggtgcatttcagactctctcctcccactttctcttccctcctctaactctttgggatcgcccccgccacacacaaacacacacactctcttcctctctctctcacacacacacacacactcactcacacctctccaggaaaagcagcagacaaatggggattgaaaaattcaaaccctccctctggtcctgggaggaaagggctgtctgaggtccgcagggggtggaggtgtgtgtgtgtgcgtgtgtgtgtgtgtatacacacgccctccctggtgtgccttttccggagcactggaaagccgtccacggcggaccacctcaagggcggccgcggcactgtcctgccccgtgccccctgccctgaacttcttcctcctgcgcccctgcccctatttgcagcctaaactcctgtacggctgccacatttcttaacatcttggagggggaggcggagtggagagaggcggagagaggaaggggggagggagccgaaataaaggtggtttccttttttggcagccagttttggttttgttgagcatgaaatctctgctcccttaaaaaattattctcggaaaaagatatcccccccgttttccaggttttgagccgcctctccttagggcctggtcgggggaggaaaagttgtaaacaaattgccaccttaaattcgcggtgcgagtctgcggagctgccgggttcattgtgtttacgaggctcgctgaaatgtgtggaatccagggaaggcgagcacccagacgggggcccgccggggccgcggccagcgccggggaaatgccgcgccggggagcagcctgcgccggcctgagcccttccctttgcactcggctgttttttacgtttaaccagaaaggaagggagaggagggaaagatccatgtggctgccctcttccgatcacaaatattgtcgtaagttgcagctggctgccccacttcctaattcagctcacacagcctctccccacgctatggaaatgcgtcgggagtgaactccggcggccgcgctcaccacgtggatccccacttactaccattctcggcgggggtccagttgggggaacccgcaatatgttgttccaaagagcgctcgcccctagcgcccgtccccgagggtgatggacagagcaggactggtttgctggctcctgaaccttgggctccatcgctgggattacgcagcccctcccttctcagctctggggtgaatattgtctgtgtcgagtggtttctctcctctctacctcggcttcctcttctgtattgcagccccagtgccccaagtcgctcagggatcccgcgggtgggggcgccctgatgtccccgaggtgcagctcccacgctgcggcccggctttatgaccgagccccccatgacgctcagatccgccggggctgcagtctcgagggcgagtccgtgtgccgcggccacccgcgcccccacggctgttgggggctcaccgggcgggtgcgcgcaagcgggcgtgggcgcgcgggccgcgactccccggcgctcactcctcccttctgcttcgtccccaggggaaggctactggccggaaagcgccgctgtggctgagagcgaagtttcagagactcttatttaaactgggttgttacattcaaaaaaactgcggcaagttcttggttgtgggcctcctcatatttggggccttcgcggtgggattaaaagcagcgaacctcgagaccaacgtggaggagctgtgggtggaaggtaagagcgcccgccgcggcgcccgcgcccccggcccggtccctcctcgcggttgatagagatattcgccagcgcacacctagagcctggctgcggccggcgaaaccccctgcccgcacccgggggattgtttattgtttgggcgcctggccgggagccaggcccgcccgacaaaggccgcgctgtaatctactcggggcgctgcgccggccgaggtgtggtgggagagagcggcgggggcgggacgggggcgcggggcccctccgggacggaccactctgcgcgccgggggccccgggcccgccgccgctgccgctgtccgcctgggccgcgccccgagaggcgccccggggctggcctctcccggccccgctccgcggcccggctttcttctctcccccacccccacccaccgcgtccaaagaaaaaggagtcggggagagccagaggccacccctccggctggctggagtgtgcgcgcccccggcggccgcggccgccgcgctggggagggccgggggcgggcgcagcgcggggctgcggaggcggcggcgtgtgtggtcctgtttttggacagctttcaactctgagggaaaaaaacatttgtctcaaggtacaaaaaacgcggggtgggggtggaaggagtggaaaaaaaaatagtaaggcacagactggaacgcatgtttgcaaagtagattaatgtttcctcgcgggtgggggctttttacatggaattattctgggggaaggctgcattttgcaggcaaagaaaagaaatgttttaagaacgcggggttgaagggacctctcagaggggttctgccagtcttttatccacgttcctcagttttggggttcttgagaccttagcaaagcagttgcagatttaaaatagatatatattttttcgaagacaaacatttttacattattttggtgacaaaggtttcaatttttttcccccgctggtttcttatttatctttttggcaagttccaaatcctgctatttggaaacactttaaccctaaacaccactttgggcatcccttaccttacaagagggtagtaggggagggtagaggtgactaggtagataattcaaaccaaaatacctggagcattcctccggacagcatgtggaagctgaagagttatgcgcataatcgcttcagtgatccctttcaggatgctgttggagaccttgaagaatatttccttaatctctccttgtttgtgaagattcagcttgcagagttttgcaagcctggggtggagttggagtatttagttgagaagtgacaattaaaataatagtctacataattaagacaaaacattcctgccttaagtagtttggtaattaatacaaaagcgttctttggattgaatccatggagtaatttacctcagttcagcgattcagtatcactggtagataaaagaagggttttatcgagcagctattatgctcctagcaacaagtttcttgagcacctatgatgtccctagcactgtgttaagcaccataagagactcaagtgtgtgcgacactgcacaatttaaaactattagaattcgatgcatttggactacatttgggttcacagctgaaatgggctgtgaaagttacaagagggtggtagggggtggggggagagaactggagacggtaggggaaggagggcattttaggcatgggcgggacaccagcaggggccccagctgaggagtttggaagtgatgtgattggctagactgtaggggcgctgccgtagattctaggacagaatagggcatgaggacagggagggccgctgccgtagattcttggacagaatagggcatgagcacagggagggccggctcatccaccacttgatccaacctgtatttgttgccatcggatgaggatgacaaaacgtgattttgtggtgttcttagcaagagttttcagtggacctttgacatgttgaaggagatagaatgtgatttattttcttaacattgcatatttctggaaataacttgctttctgcataacacgacccatttcaagtcctggaaggagatgcagtgtgcttcctagcagttttatgggagctgccagttgtgtagtgagagggagtaaagtttttctttttcaatggactggatgaggacacttctgaggggcacactcactttgcttacttttggcagtttcagcatgactcactggtcttgggccaaatgttatttactcagtactaatactgtcacttgttgagcaactgccatgtaccaggcatttgttatatattatcgcatgccttacgattcaataaaatagctattattatcttttttttttttttttttttttttttttttttttttttttttgagacggagtctcgctctgtcgcccaggccagactgcggactgcagtggcgcaatctcggctcactgcaagctccacttcctgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccctattttgtttttataacatgatagtggatgtaatgtctttatgatgaggattgtcccattttacagatgggaaactatgggttgtctgtttgtctccaggtctactttgcctactaatgacataagtcacttgttctgttgccctgtccttgatctctcattcattcattcattcattcattctgtgctagattctccccctagagaagaagcgacagtttcataaatgcagatagacccaactagagaggtgactatacaacagtagccatatagatttaatttcattttcaaagcagaagtgattgtgtagagatcttgtttgtgacagagtgagctgagtgttcacgtcagcagtaagtagtgtgagcatttgcattcaggagtaagatttccctgctgtcctatacctcagtggacgtccgtgaattgaagtgagtaatagctccacatttaagttcattttgtgcatcagtctcatagataaattcctcatcactatcagcaaagaaatctgttagatgctgctttcttggtaggttgttcttaaggttctagacatgaatttttatatgaactgggcatcaacagagtgcttgctacacaaacgaaatggatagaggtagcatattgccctagttagaaaacaacagaagagaggaaagtatgagattttaaggggagagggatgctgagggatagtttggaagaagctccttctcctgcggtttaaaatcacaggagcgtgtgtgtgtatgcacattgtcttattctgcatatcctcctatctgccaaaggcatgttaatgttatctttttaatacagccagcaaattaaagggatccactcgaaaggctgtgtcttcaaattgactttcatgctttgtttaacatgctggggaaatgtggctttataaagagttggctttataaagagggcagaatacatcagccacaatgtgcagactaatatatgctagatgagaaagagtccgtgtgtgtgcatgcgcgtgtgtgtgcgtgtgtaggaattggtgataagaattattgtattctgttgttaaaatgtccatataatgattgccttttaataaactgtcaattatatgttaagataccagcttgagttttagggttatagtgtttgctaatcaaaattgtcaaatcagtttcacatatgtgcggagtaatgaggtttgatagagaatatgttgactgatgtagtggacatctgctgttgcagaaacacatgaccatggcagcaaggtgggggagaaactaattgtcattgtataaaaatagaagcgttcattctgcacatgttctcagtacctactctgcaccaccattgcagaaagctctccagagagatgttggtgaaaagccacagtctctgccctcaggggaactccatccaaacattcagcaggtacacggactggtgtgtgggtagttaacatgaaggacagcagtgccaagatggcaggaaaatcggggacatcttccaggatggcctcaagggtggaggcctgagctaggcacaggggctggaggggagagggtagggggcattcagggcagaaggaacagcataggcaaaggctcagagaacaggaatgtggcctcctggagggcagcctccctttgtgggtcgttgaggttgggtgggagcctggaagctgggctcgtccctcagggccatttcacagaagtctccctgcttggctgggggctgtcagctcttcctactagccatccctgtctcacgagtgtgctcacgtgttggcctctagtgttttccctggagccacgaatgcttcacctggatctcacctttctgggcacagactggtgttatttttaatcttgcctgtttctttgttggtatttcctttttgttaagcagttttaacccatctccctcagtatctatttcttttcatatcagccccctcactgcagccctccctcagaacaagtttcttgtcaagcttctccagcgatagaacattcatttatttctttgagctttttgttttttctaaaagcttgcccagctcctggcctccttttctgcagagggcagcagccttgtaatgaatccatattgctttggtgtttggatgttaacattgctgcaggccagcgtcaaggaagaaggaggcaagttgtgactttgacttttttttttttttaagaaaaatagaagaaattgttttttaaaatacatgcttggtatgtgtaagtgctttcaagtttgtaccttgctagtgttaatgctttcagtgttcttgaactcaggcaatcagttgtaggagataactgagtttcaccactccttcaatttggagcaaggtccctcaacctcaggactattgacattggggctggatcatcctctgttgttgggggctgttctgtgcactataagatgtttagcagcatcctggtttctacctactagatgccagtagcaccctcccagttgtgacaaccagaatgtttccagtcattgccagatgtcactgggggagagggtgtcccaaatcatcacgattgagacccactggtttagatgataaaaagattttgattggaagactcatgctgtgcagatggaagacgtttattatgatggagttgtggctacaatgcattgcagagatgcaagagtattaatcttctttaagataactcccccatcccattcctgtatttatagacccagataactatttttcttagaatgacattaaatgtcaaagaaaaacaccgtgatgggtcgaaagaggaaagaggccatggaagaaagaaataatttatatttgggtaccttttggtggggggtatctaaatttgcagaaagactggttgcacctcaaccccagtagcctgaaggtttttagcatggttgactttctgaaaagctttaatagaagacaccactgcctttgtgcatattgaaataacagctccttcagaagcccggctagtgtgtgaactccttggtggtctcattctcctccagatgtttctgtcacaaaccactcccccttgtccccccaggagcccttgtggagcagaaccaagggacagcaagggaccagtcccagccagaagggcagaatggattctgctgtcactgcccttccacagtgagcacctggtttcttttctctggggttgccattgtgggggcatttcacaagggacaggaagaaaaatgcagctcctttctgctccctggacccccatacatggtttagaagggagaatatgtttataggtatttattagtttctactactgggtatgcacatttaggtggaagcagttgtgatatttctttcatatttggagaaaaacgattggaaatgagaagccaccaatgattgtttaaaaaagaacaaatagccctcaggtgtcttctacacagggcagttgaccctgcgaatcctccctatctctttgctgtctgccattcttgacccttcaccaagttgagaggacaggttgggtaatggggtgtttcagatacataggatgccgctcagaggttccagaagatgacagcttccagatgtatgaacagcctgatccgtgtgtttagattatgcaccacagctgatgccgagattaatgctggctggacaagccatgggagatttgtctggtttccacaatgccatgtttatgtttcatttgagtttctccttcaaagctgacctgcctgtgaccctgggctctgtccactcgggcaagctgaaactcgggcaagctgaaactcgtctgccctgttctcctgcttgtattggggtaggggagagtcacaattgggcggagaaaggggcgctttggtggccgccttctgcctcaccatgccagctatctgcccagatttagcccctgatgagtgttcagttctcctcggggatgtgtttcactgtaaaaggttttttggcttttaaaacaaatatggaaatattataaaataactgtaaaggggaccagatttaatagaagccactttgcataaatgtagtattttaacttctggcactcttttgacactatttagggggagaaaaagttgagggaatttcagctatgtcagacacccccacataataaccatggctgtgtcatcctattataatactattaatgtttcctgtgcccctcgctggctcccagaacctctgctgcctcctccctctctcccctgcccccttccctcctgccccttctcccgcccacccactctaacctgcccacgtcatttccttccaagtggatctctgcagttactagtgtgaactctgaaatggtagtaagtctggctaatcacaacctaagaggagggaatgagaacatttattatttcaagccagctgtgggagatactagatacagagctgccatttgcatccttggcattcttattttggttagggttggttccaagaccacactctttgatgggctcttggcagaagatgtgattcccctgttgaagaatcatgactttttttaatggactgctgaggcaactttaaaatccattgacaagtttcttcaacccataccacaccgccagagagagtgggaaataagtcttggtcccctgtgcttaccgtggcgcatggacagtggaagaactgggttccctagcatttctgagacagtgagtggagcacaaaccatggctttccctcccaggtgccccacttcaggtcttccttgcttagaactcccaccaaagccagtgggcacatggagcttggctagattgaagacgatggaattccttatccagattttggccgactctgaagctttatgtgttccatcctcacgagaggtcttggttgacatccagagaccagagggccttgctggtgagggcacttctgcctgcaggaggaggaagctctgcgggattctctcagtgggaacattccttgaatacacagatataatcagggtcatcaaaatataaacacttctgttactttctcttctaatctttgaatttgggtgcttatgataatactcactggattttttttttctctttaaattggatttagggcactttttttcctttacactgcaatgtctaatgccttccgtacagcagaatactgccattgagaaactgctgggctgtggtggaggattttaggagcctggtgtacaggaagatgcgccccatccttagtccctccaggagggaatatgactgttccatttggggtttagagcatggctggaaaaacagctgtcatgtgttagggaaactgtattgttggtgggcagtcagatacctgctaatgaatggcaagtttgttgttggagagatttataatgtgggggcaggaccttatttctaatccagaaacccaggtgtgagttggcccaaatagtccactctgcatgtgggtcccataatgccactgctatgaaagcaaggctagtgagaagcaagtccccataccacagcgcaagctttatttattattgttcggataaatgaaaagaagcagtagacttacccaatttagcaaccaagatagttttcagttattaatgagacatgtataaaagtaagcaaaaggaactggaaatgggctttgttaagtcaatgtgcatgattttttttcccccttaaatcattgcttaagggcttctggcccctagctcaggaatcctgcccaaaaatgccagatgtattacacagtcaagtattgcaatgtaaacaccactccgtgccacgaggcagggtggtccatctcctatttaagaagagtgcgagattgagaggatggggggtgggggggtaaaccctctgctctgtagtgagcccagtggtggttgtgtcatttgtgtttttggctgcacagtcaagatttgaaactacaaaacccagataaacacattggaatggcaaagaatagcaacttgaattgggatgccaaacaaatcaagatgtggcttggatagtgttgcttcattgggtcagataagtttataaaatgtgggacagaatggagggtggggagagacaggaagagcgctcttgtcacagcagcatctttaagttgcatgagcatcctgttattcatttgctggtggttaaatggaaaggtgagtggaagcgaggcggagagagcccgagcctccaagatgacctcctccgtgagaggaagccctcccgggtgggtgcaccttccaagcctggaaaagctgatgggttgcaaagccagtctggtgttctgtggcactgagggatgtagcccacgatatgttctgaagtaaatggctttccagttttctcctgctttctgcatttctgaagtaagaagtctgctaatgatgattgttcggagctgagaaagtcactggcactttgggccaccctccggtataacgtgtcgtacgatgctgccagcaatgacaggaaattggagtacccaaatattacctatccttccagatagcctaacatgaagcagccctgagagtgactacactcagggtttttaaaaattaatacgaacatgttttgtgagttaggaagaaataggatttcagtttgaatttgcggaattgttgaaatacctgcgccaatattctcctgattaagaaatttattctacagagtgatctgagaaaacggctttaagaaatagtgatgataaaaacaggaattttgagatgaaggaaaatgtaggcttcttgactccctctttctcctcccaccgctgttttttttccttttgtgaattcttccacattcccacccccactccctgatttcctctgctcctttactgggaaaccagcaaccttcacaaaatagactcaaacatcacacagcctctttttaaatagaggctttctaagagaaaataacaaaagaatttttcctagctgcctaagaaattctttcttcttactttattttcctcatgagatttacacattgttgtaataagtaaaaaatgataaatatgataccatcattttggtctacacatgatccagaaaaaaaaattgcacatgtttaataccttgcttaattgcaaacaaagcttgcaatttgagagaattgggattagggctaaatatctattaactgctctttctagagatttggggactaggatgacccttttttttttcttttttttttttaataggctacagtttcacatggctcccaaaagaaatctctctccctttttgtttcccacaccaattgatttatcattttgcttgcaagcagatccagaatgttagggcaagaacacactcacttttttctcgtaacttttttgttgcggaccgaaaccagttgacttgaataaatttttctgaggcctgaatgtagccagcaacgtggaaaaatcttgccattcagggccgtctgaatgtacatttcagccacacagtaagctgcattgggaggcgctggactgctaaatgcaaccatttttcgcagcagcgagttagacaggtgattagcataattagcaccgagcagctatacatatggtaatgctgagtgtgtaaatgccagctgcggtgtaaaatttatgtcaggggtcggcagggctgtgcgaaagtattgtgttccagctgcaggtcagggccgccaaagcttacctccttttctcttgttggtgaatacgccaaacagaagagaaagacagaaatataacagatacggctttgatgggacagcccagttctgtgcaagagggactgctctataaaacacacacactcacacacacttgcagaaatcccattactttctgtagaaatcgtgtgtgtacttcatgcagggtccagatgagtggggccgggatggaggtaggtaaatgattccctgggaggaaaaaattcagtcattgaagttttaaaaggagttaaaagttctgttttgtttttcttcccacccatccttttaccccccacaccattccctccaccactctcctgcccacattgtcttatttcacattagatggggggatatccttgctggatgggatagcattggtcctttagaagaggaggaactaaccgttctggaggtgttagccttgaacaaaatataaggtttcagcagtgcaactcttcccaaactgcttgactagctttagaaaggggctaaaaaaagcttttgtgggctttttgttcttcctctgggtagctatacttactcgcctacctatacttactccccctacctccactgtcagacccaccaaattctttatagatttaaaaaaaaaaaaagaatcacatgggtttcttgtttgaaaggatagtcttgaatggctcacatactttgaaatgcatgatgcgggaatttattgctatgatgagtttatacattttgcacttgaggtattttctctgccacgcccccacctccacccttccgttttttggttggtggctataagaagtggtggtgttgacagaatatagttcacaccatgctgacccggtgatgaaacttcctcacggcaccagggggtccttatgtactggcccctaatccagctaatcctgatggcaacaaaatcatgaaagtggcccccagtgacgtgagtctccctgcacagatgcagagggaaggaacagtgcaggagataaatgaggccagcgtggtattcaccggaggccagggagcctgcgtgcgaaggtggagactcgcattgtcttctcccccatgtcggctcaagtgggaggccaatgaagagaggcccaggctggataatggcaagaagactgttcagagctgagaggtgatgtcagccccacagaagctgagagaaggaaactggggttaatgttatgcaatgccttgagtgctgtgatggagagcctgccgtggaagcacttgggttttgttgttgttgttgggtttctttctgtttctatttttttaatgaagacttcaggaggtttcaactaagcttgatgaaaacacgctgtgttggttcctgggttctgctgcctgctgctgctggagtgtggcctctgagccagcgcgcgctcgtcatcacacctctgggcccgcccacttcctgcagagcgacgggacctgggtgtgcatctgcctgcctgttgcgcttgagcggcctggttgcacctgaggttgtggtagaacctgggacagttcccagcttggaaagtacgctgttccggatggaagacatttccgcctgtgctctgcaggaagtcagcaacgttcttgggcctgtctcaggtggcgcggtgcacagccctcatttgccagtgtggcaagtaaccaatgaagaaggggcctcctggcagatgcagcgtcgtctcgggaactcgtggctccttgggagctggtgggagctgcgacaggcagcaggtccagcgtgggcagagtgccgggctgacctgattgcatctgacagggcgagcaggtctgcgtcagggaaagccattcagagggagcagcctgggcaaggctgataacagatggcactgccagcagctggtgggggcggggaagcttgcttccggcgcatgcgcgttgcagaaccccgcgcgagcgcctgcgcactacctgcgcccagcctgccacatgccccgtgcggtcccgtgcggttccgtgggcaggtgggggtgtccttttctcgttatgctttggtggttgacctctcactgggctcacttcatgaaggcacattctctttgggctgattgtagcgtttttcccatggcaacacgcaggggcacaatctcataaggaaaaaaacggcccagaaacttggacagcggttggctcctcctgaataaaagagatgggtgggagaggactaaaattcacagcccgtgaattttcagccaattgctcacattactgaattttcttttttttttgtttttcgattataaaaaagaggcagtgctaataattttgcagttacacccgcttagagatttgtactttttcggcctcacagtaagtaaatgcaacattgaaaggaggacctgagcaactaaaagaaaaagaaaatctctctagaggctgttagataattcatgggattaatcaaatgcaaatgcaacgcttgcatatacacagaccctattttcctaattggactctgagtgggtacttttcaggctgaaagggtcatttgttttgactgattgactttttgaggctttggggggaattaataacttatacagatgactgcctgttcccaaggctctgtattgagactgcgcattcatttattcattcagcacacttggtgtgccatgcccctggtgcccctctgcgtccctcacatacggaggtgcatgggcaggacttgaattttgtggaaaataacttcggttggtccctcagatcatctttatgtggatcacaataagcttttgctaaaaggtttttgtgtgaatgacacagaagtttcttaagctctgtgcagaaggctgtgctgaggaggggcacagcagggaagcgtgatgagggaaacatctcttgtctacagtggattggaatcatattggcctagaagtatgggcctctgggtcagctggcatcccacctgagctagctagcctttcttcactttggaaagttaagcctggcttgatttggggatgagaggatcagcagagcttgatcaaaaacttatttgcagaagtgatgcttcttttgcctttcaaagaattggcttaagccttggatttctggagaataagatggcagaatttgcaggtaaagaggcttagggctgcatgttctctgtgtgccttcatctgccccctctaagacaggactctctggggaccagaaggaaaatgaggcctgtccactgtgacccagggagccctactgaaaaaaatgcatctcctcactggctgagaaaagtctttgatgcaacatggttgtactttaaataaaaatacggattattgtcatatttcacgcattctaaaattctagctgtgagcgtaatacaggttgagtatcccttatacaaaatgtttgggaccagaagtgtatgggattttggatttttttaagggggggtgatattcacacataagcatgatgagaaagcttgaggatgggacccaactctaaacatgaaatttgtttatgtttcaactactttatgcatctagcctgaagggaattttatacgatattttaagataattgtgtgcctgaaacaatgttggtgtatattgaactgtctgaaagcaaagatgtcactatctcagccacccatgtggacaatctggttgtgtggcattcccatccttcctgacttaaaatttatatgctgccgataagcaatcattttcttaaactaattcacacctacgtacttaccagtaaaaaaaccacgacattccattaatgcagtgaaaaaaataatgtgttcagggtacccaagcagcacaatagcatcaccagatacctgcatcagtggctaaacaacagcagcttccggtctccacctgcaatgctgtgttttcactgagaggttactgtacaccatgttttatttttttaggtgagaagaaacatcagaagcagttgaaggatcagggagtgggttgtgtaggggcgaggagacatctgctggatggcttttcagaatgtttcctccagcatcaccggcctcattaacaatagtttctgtcttaagacgtctctctttgattttatgaattgacatgatttcctgtttggttgtgaatgcacgatgcgttagttttcaataagcccatcacacatttttaccaggtctgctatagttaactttttgtgcagttgtaacatgtcatcttcatggttgtgagcctgttttgactgtgactgattacatgaggtcagatgtggaattttccacttggagcatcatattggcactcaggaaggttttggagcatttgagaccttcagattagggatgctcaacctgtctagtgtgctctgtggaatttggcactcctaggctacctcctgctgtactcatgttctcagctggtgctcagaaaccatgaggctcgcagggtagttggactctgggttaaagggtcagttctgcctcttactaataaggtgggcaaccttgggcaagccacccaaactctgcagctatgccccctcccctgtaacatcaggacagtgaaacaatgctctgtgacaaaggcatgggtgaactgacacttgctccgctcttgggtaagggtctgggaaatttcacacacattgtcagctaaggctctcacagtcctgtgaggttcatcacagcatttgttatccctgttatcctcagcaaccaggaaaggtggctgaggctcagctgtgtcagggacttgtcctagatctctctgccactagtggcaatgctggggtcttccggtttccattctcactgcctgtctggtacagctggccccagcttgatggaggagcagcaatgatcaaaatatttccaaagacgtaaagaatatggtctcaaagggctttgcaaatggaaagcaccacccaagtataaaacgttattttgtgtctcaggatcctgagataatgatttttcttgagtcttgttcagaggggttgttattttattgttaatataaaaggacttcagaatatgatgaagatgcatatgattatttttatagtttattagtattattgatacataatactgtatatatttgtggtgtacatgtgatgttttgatataagcatatgatgtgtaatgatcagatgagggtaattggaatgtccatcacctcaaacacattcatcatttttttgtgttgagaacattctaaatcttctcttctagctattttgaaatatacagtgtattgttgttaactgtagtctccctactatgctatggaacactagaatgtattctttctacttaactgtatgttggtactcattaacccacctctcttcatttcccctctcattcttcccagcctctgataaccctcattctactctctaagtccatcagattaatcttttttacctctcatatatgagtgagtacagagatgcctatttttttttgcattgagtttgtttttaattcatatctctaccaagtagctctcatatctatctttcttttgtgtgtgtgtgtgtgtgtgtgtgtgtgtgtgtgtgtcacatagaaggttttgtttttctgggttttgttttgttttgaagtggggtctcattctattgcccaggctggagtgcagtgacacagtcacagtgcactgcaccccccaactcctgggctcaagcgatcctcacacttcagcctctgaagtagctagaactaaaggcagcctccactactccacagctatttctttaactttttatttgtagagataaatacgtttatctgtgttgcccaggctggtctcgactcctggccttaagtgatccttccaccttggcctcccaaagtgctgggattacaggcatgagccaccatgtccaaccatagaagcttttaattatactctttcattgtagaaaaattgatagaaataaactcaaaaaagcaaagcgggtaaatattatccatagtccggccaggtgcagtggctcacgcctgtaatcccagcactttgggaggccgaggtgggcggatcacaaggtcaggagatcgagaccatcctggctaacacagtgaaaccccatctctactaaaaatacaaaaaaaattagccaggcctggtggcgggcgcctgtagtcccagctacttgggaggcttaggcaggagaatggcgtgaaccctggaggcagagcttgcagtgagctgagatggggccactgcactccagcctgggcgacagagcaagactccgtcacaaaaaaaaaaaaaaaattatccatagcctaccacacagcaaaaatggctattaacttggttccctatgcctacatctacataacagatactagatttaatagttattttcttctgcactatttcacgaacattagattattttaaaaatatgttaggactttgatcctgacagtggaaaaaagtcatgcctgaaatcccagcactttgggaggccaaggcaggaggattacttaagctgaggagtttgagaccagcttgggcaacatagtgaggcccccgtatctacccaaaaaaatgaaaagttagctgggcatggtggtgtgtgcctgtagttggagttatctggggtattaggggaggaggatcgcttaagcccaggaggttgaggctgcggtgagcagtgattgtgccactgcactccagcctgggcaacagagcaagaccttgtctttaaataaaagaaaaagaagggggaaacctatttctccaagaccatgaccttcatctcagtcatctaaaatgggttctgtgggacttcctgagctcaaagcttcacacttagggatgataaggaagacagcaagataatgaacagctttcagctgtcattcagcagtgatttaggataggcgtttgctagggtagcaaatgtcagacgagtcagtaaaattgctctatcctgaagggtttctgtttctggcagcttgctccagagtgccttgggtcaatactgatgtcagaatatatgagtgtcgtgcactttatgacacagttatacccgagagggagctgattctaccagttcacatggtgaatgacacaagacttaattatggtctcaccaagctccgccacacttgcctcccttctctccctgcaacaagccaaacctgagggctttgcagttgctggttcttctacccagaacgctgtttctccagctcttcaaagtcacctaaatgttgtttccctttaggtcttccctgctatcccatttgaaatatcttttccccttccccactacccaacatggcatcaccatcttcgttttctgcatcacacttgtgctatgaaggtatcttattgttttgcttggctgttgcctgttcccttcactgaccagcatgcctcttggagagagactacgctgactggtttcgtccaccacatccccactgcctggtcaaagtacccacccacccacccacaaatgcttgctggatgaatgaatcatgttacagctattggtcagataaaatgggtggcaactaaacttcattaacgtttttgaatcattttgcaaaacattcatatttggttaaatataagatgaaatattagataaaatataaccatataatatgtaaaagtgaattgcatttaatagtatatatggcataggtggttgagaaaaccgtagacttcagagtctcaactctagttgcttggccaccctatcatgatagtcttagttgaaacatcacattggcaaaatatttgtgttcatcattagagcaaagagatgggggagcgacaggttttccaattccagagaaaggacaaatctatacctttattaaaatgtttatgtaaatatacatcttaaaacatctaaaaaggggacatgatggagaccctgaggtacattttatatcttgtagaaaataaatgccatttctgaaacaccctgtgataaaataactgctccattatgaggttggtctggaagagtctggtaaaaatttcttagcagcacaaactttgtgtttggttttgccatttttgtgcacactgcactctcaggtcacttcgaggattatatagttaacaggcggctgctctgccaaggctcgaggggcagaatcttgctgaagccaggctgtctgtcatggactgctcctcactataacggcacctaggtcaccataataagggtggggggtgtgctttgttgctgctgttttttaatttcatgggtttaagtcacctgatatgttaagcttcagtaaaggtgcaggaggtgcaactttgtaatcagaatgaaaaaccacgtgcatgctgatgttcgaggaggcagtaacaatgggaatgatttctgggtttgtggcattagatgtgataacacaaagaaagggtgagcaaaagctgcattgagctccttggtgggcagctctgcagctgtgacctctggactcataactggctttttagcctcatacagcttctgcacttctaggggatgtgggctggctcaaattcagttaattggttggaagtctgcgtggtgttaagtactgtgctttctgctagagtgagcaagcccctgccttcaggcagctttcccagggcttgcagaaaaggccagccgagtaggtagatgcttctctgtgcaagttgtttcccagtgttttcctgtgatttttccttaacttcacattgatggcaaggccctcggcaatgccccctggcatttttcaaagttttctgcacacagaagtcttattcctttttattaaccaactgatatattcctccctccttcccgaccccacacgcagtatgttaaacctcacctcacggagctcctggccttggcatccgcgtggacctgcacagagggtcctgccaggcggtgaatttgtactggttttcctaatgagggcattatttgctttttagcctgcccccttgtttataaaatatgccaagagaactaaacaacccgctgttcccagtccttatttacttgtccaaaccaaatgtttatgtccttgaatactaaacttgaagaaaaaggattcctcctcgtgggaaagctatgatcactgagtttttttctaaactgtcgctgcaggcctgcttttcagtcaacatgcaacttatcaatgactttaggaacaattcagacccgcttcctttgccctagactgtggagtgggggagctctaggttcctgggtctgtaactattagtatctccctgagacacctcccagcccaggtgcagcattcggagcttggcttggcccagaaccgttcattgaacataaccttgggcctaaggatgtaaaaaggagaggaagagaaaaggcatttggcagtcacagtgttgatggttttctttggtcacctgctcctgcgctggctttgggcataccccgtgctggccagcttgctggaaagctgggttggtgtgaggcaggacttctcggcctgggcagcaggttcagggtcacctggggtgcttccggaaaacaggctgctcacccccagctgactgagaacctggcacctgtgttttctggatgctgaccaggtgcgtctgatggaaccaggttccagggactgcccatgatggctgtggctgatacagtttgttcctctcaattgggatttccccatgggcatctgacttgaatcactttatttatcttctcctggcaagttttgtgctgaacacacactggccacctttccttgtttctttttccacagaggagaaacatttccttgttacctttgccagtttacttaggagaagagtgtaaattgaagccatttatcttgaaatctgctgcaggggcgcattgtttatacaagtgaaagaaattgtaacagggttatttggtctggtctgcaaggaaaaggtgtctcaagttttcctttctgccccccactttcgtcacacagtgtattagttagaattaggtttgtgtctgtgacaggcgggacagccattaatgggcttagtgagtattttttatgtttactattggtgaagttaacattgccattaataaagtgaatgttactgctcacacatcagccagtctcatgactagagttcatctattgtgtatcctatggcaggtagtcagataacagataaaacatgagtttgcagtgattttgctattctaattaaacctgtacatatttgtcagttggaggacgagtaagtcgtgaattaaattatactcgccagaagattggagaagaggctatgtttaatcctcaactcatgatacagacccctaaagaagaaggtgctaatgtcctgaccacagaagcgctcctacaacacctggactcggcactccaggccagccgtgtccatgtatacatgtacaacaggtaaggcccgccgcgtccacctactaatgagcagcaggtaaggcggtgggagaaggctgctggtttaggccccgttattttagtaaatgaagttgccctggaaatgcacctggagtattagttcaaatagctttgcaatgaaaggatttttttcatcttgatttatcgtttctcgagtttggtaacaaagcaagtgcattacttgataatgtattgccagtaagtatcttggcactgtccccaaaagaggatgacgctgagccacttggggactatacacccaggctgtaagatgtttcattgcttaagagatttttggagagtgatattttgaattgcccttgctgaacttaaaaacaatgcgaaaaagaagagctgcttcttgtgactttgtactcttagttgttttgtgtcacacagcacgttgggggctgagccaccatgggaattgttgctgacacctttttatttatttttttgagacagagcctcactctgttgcccaggctggagtgcagtgatgtaatctcggctcactgcaacctctgcctcccgggtttaagcgattctcatgcctcagcttcccaaggagctggggctagaggcgcccaccaccacacctggctaatttttgtattttcagtagagatggggtttcaccattttggccaggctagtctcgaactcctgacctcaagtgatccgcctgcctcagcctcccaaagtgctggggttacaagcgtgagccaccatgcctggcccgctgacacctttttaaaaagtcatgttttagcatcgtattgctaatttcacaatgctgtgagtagagtaaagcattaggaagagttcaaaactcggtcctttttcttatgtatctagactgttggttactacagggcaggggccacctcttgtctgttttcaccccagcattgagaacagagtgggcattcagtttgttgattgcttttgaggttggcatcactaaaccccatttccaggggacctctgagttgatgtcacccaaccccatcacctcccctaggctttcagcacctcttcactgaggcctttgttagtactgtttatttttcctggaagaggtgaggaagtagcagtataggcatattcagcagccagggagaaccaaagaagagaataaaattaagatttttcttgtcccttctctaacctctgcttacattccagctcgacaggatcctagggacaagcacagccagggtcgagtcacatgtctttggcacattcttgtttgtttatctgtaacctgggcttatggtcatgtgtgctcgtatccttcatggttcccagaacaagaccagatacatgaatgcaagggccttagaaaatgtaaaatcatgtttctgccaggcaaacttgccctgttggtctgtaacaagtattgtagggaagcaactacctggtacagttttgcagaatgtgaagtggcatccctgtggggagccaggttgtcctggggccactgcaagcatggccaatgcattcttagtgtttgtcacctacatgcttcaggagaagatgtgagggtctgtgccattaaattacactcttaagctgagatgggtccagagtaattttatgcattggaatgaactttaagaacatgtagttcaacccatcccatgttttaggtgagaaaactgggaattagactaattagagcatttgtccaagttcatgcagttatgaaatgagagagccaggtttcaaagcctttttctggcttcaagcccattcatttttctaatgcacaatgatgtccttctcaagtgtaaatgagagttgatcaagtaatataaagtgagaacctactcatgccggatagtctatgttgatgctgtatagaattaaaatataaatatgtccatggatgtgtggctaggtggaggatggcggagatatgggtaagttaggcacagctctcaaagagaggaaagacatcagtatagatagacatggacaaaggaaacgtgggcggtccccatgacagctgcgccaggtttcagcaggagatctgcccagccagggctctagagttcaggagaagggggtcattttcaacaagcttaacatgggggtgacatttcaacagggcgctgagagacaggaaggcacagggaagaggacacagtaagctccttgggtgaagacaggcctcccttaggaggccaagaatagagcagttgggagggaagtttttactgtgtttctttcttttttttttttttttttccttcccttggacttttccatccccactcttccagattttcttttttttttcgttagactgatctccctaacacacctggaaagatatgggcttatataacacttttctagagacaataaaactcccgattaattcttttttttttgagagatagggtcttgcttatctaggctggagtgcagtggagcaatcacagcttactgcagcctcgaactcctgggctcaagtgatcctccacctctgcctcccgagtagctgggactatatatgtgtggcaccacgcctggcaattttttttttcatttttttgtagagatagagtcactctctgtcacccaggctggagggcagtggcataatcacagctcactgcctcagccttccaggatcaagtaattttcttgtctgtatagctgagatgacaggcatgcaccactgcgcccagctaatttttaaagtttatttatttatttattcattcattcatttcattcattcattatagagatagggtctcactttgctgcccaggctgatttcaaactcctggcttcacgctgtcctcctgcctgcacctcccaaagtgctgggattacaggcgtgagctgcagcacccagcactaaattaattcttgatctgtatatttattcacatttctgtaagataggaatagaagagactgtgaagttagaatttgacccacctggcacatgttgttgtttataatattaatggttaagagcaggagagttcattttgaatactacctattaaggacatgatctgccagtaaaacatctccgcacctgagtatgtgtcaatactttgcaaacctaatttaatccccgcctattttgaatttatgataattcagctgggctgccctgaagttgtttaatgtatgtagaaaagcagattaatggtgtaaacaagattattgggagaacacttaagaaaacaagacttttcaagtactttggtagcattcatttacataaacaccctacatgttaattacgattgagtattatctattcattaaagcaagcttgctgggtctctacttggcaaaagctctgctcgttttgacagatgcttaaaagtgatcgaaattgcagttctttttatatgttctatcatttcgaatttgcactaattttcttattacagtgagaaatttttgtctctgcttttcattttattaggcagtggaaattggaacatttgtgttacaaatcaggagagcttatcacagaaacaggttacatggatcaggtatattttcttcttgttggcaaaaatttctcaggaacaccccagtagtgtgccttaacctaacgcatggcctcttctttttaactttgacagataatagaatatctttacccttgtttgattattacacctttggactgcttctgggaaggggcgaaattacagtctgggacagcatacctcctgtaagtgtgtgatcatgctttctgatgtctgtgacttctctaggactcagtgtttctaatgttgcttatcattgtttgttccatttcagttttgaacaagaaataaaaacattgaaattccatgtgcattttttgagtgaatagtcgggggagaggggatttcagcagaatgttttttctcttttcaaaaaacaattctgtaggcgtcattaaatttcattcacttttaccaacctatttatatgcaaataaaatgcaaattattgcagagtacgtgtgattttttaactctttaaaatgttcgtagtttcgaagacgaggcatttagacacttattttttaaggagagactttattagtgagtgggttcacatcagaatgaaatattttgagtttgagatcttcctgtgaagtaaaaagctaacctttctttagatcacactaatggcatttaagggggaaatccactatctgtgaaggccacagtagggaatgtttcaccgtttagcagttggatattatatataccgcatcatttagagaataattgttaatcagcatagacattttcagttaattatgcaagagtgagagcttgggttattgtttctgtcctggcttgcacatatctgcaaaagcaatttgcagtggccatcccctgaccttcagtgtacagttttagatgggaaataaggggttttgtgttttctggttgggagattccgaataatgacttcagcctggttaagcagctacagtgtggtttgaaccacataacaggcagccaaaagcctttctggtctgtcaacagcacagattcaaaagtgagtgtggggggaagtggctgtgatggtggtgatggcagaggctgcagggggcttgcccattccattgccaggctgtcagagagcgctcataagtatgggattgttaaaggtagcagagatcaaatattttgattgatgacatttacaaagtatttatttggctgccaagtgctaatttgatattgtgacgccctcttttttaaaaaaaaaaaaatttaattttggatctgtctttttggctaattttgtatgtagatattaaatatacaatatcagcaattaaagattattcagtgccctcattaacttatgagtcctctctctgcttagcatcttcagtcatgaattattttcctgacctaagtttttatgcagatatgctggaaaggagcatttgttttgatgccagagtcccagaactgcagcattgccaggaacctcctacaaggtggatgcagtgggcgcagccgtgttactttacgatgcgtttagaaggctcttttcatggtctcgtctcctaatttcttttgcagaggtaaacctcctttgcggtggacaaacttcgaccctttggaattcctggaagagttaaagaaaataaactatcaagtggacagctgggaggaaatgctgaataaggctgaggttggtcatggttacatggaccgcccctgcctcaatccggccgatccagactgccccgccacagcccccaacaaaaattcaaccaaagtgagtaccagcagtgagcgctctcagaagggtggagagcaaaacacttttttgtgtccattcatcgttcactttattattttcatttttaacatttcattctccatgatcgtctttgtctatggaactaagtttatttatagagctaaattttgctgtaagatttgccatacacctcccattagccttgctattaatgttctctctgaaacacacaagcccttaatgcactggattttaacaaggcatgtgacctgcctactaattcccataattatgtggctgtctttttattttagcctcttgatatggcccttgttttgaatggtggatgtcatggcttatccagaaagtatatgcactggcaggaggagttgattgtgggtggcacagtcaagaacagcactggaaaactcgtcaggtaagccagctcccgaggacaatctatgccctgtagtcttcttcctttcctcaaaagccacttccctcctgacatcggcaagtgtatttgtattaatacttaaaccgttatcctcgctagccaggtggcgtcatagaatattaagaaatagtagcagactgcaacagcttgaaaaagcaccttgtggggaatttttcaagaatggatttgtagttttcatcaggaccatagttcctgactatagggtttaaggagatctgcccattaggtagagaatgggagcaggtgtaaagtaggccgggacacactggtcagagagagttggtggtgcgagctgcacagtgtcatagactgttagcaaaagatggagagccagcggctcaataagaattatcctttgtgcacttgtggactctggtcagtcaggtgtctggcgtttcgtgtaagatttctcccttgggaacgccaccgtctgggatttctgcacagcaagtatgcgtcactgagcccggtgtatctgttcaagctatctgctcctctccccttcctgtttcatggaatggaaactgtgaaaggaaattgcctattaatttaattttggggtatatctgtaaaaggtttgtaaggttatacctttaatatggtacatcaatctattaaataaaataaccatcctggtcccattttaatggaaacatgtgctcacagagaaggaaaagaattgcagccagtgagttgggggagctggctggggatttgaggcagtggaaactgcttcctgggaatactgatgatgtgccttcccttggactgtgctgcagcgcccatgccctgcagaccatgttccagttaatgactcccaagcaaatgtacgagcacttcaaggggtacgagtatgtctcacacatcaactggaacgaggacaaagcggcagccatcctggaggcctggcagaggacatatgtggaggtaaacccaccttcgaatcggcgtgcagactcgctggttatgcaattcttcattttcaccattatttaaaacatttctttccattcgctttattttcaaaacattgcaattctgggaacttgatgggatgaaaacttttttcctcttctttgatattgaaaatcctgggataatatctaaaagactagttctaattggggtgccctgagacatgtttagtaaccagttgtgaattccagaactaaagcacataacagtgtttaaaaaaacacttggttagcaaaggtgtctggaaaacattagcaaggcatagaatctgttcacagctgcaggccccttggtcaggtcatgtgacaggagctggcctttgtttcccaagcagtgacctctactgagctgcatgtgaccttttaaaagtggccagcaaatgaacagggggaggagggccataagaatgagttgttttggattagtggggagccgatgggaccgcaagtcaattgtgaagttaacggagaagagacgagcagtctcagccctggagtaaatacagcactcagagtgaactccagcgacaccagccctcagcagcacccactgccactgattagccatggcccttgtcctaaaacagggttctttaatgttagcccaacttcagcttctctccctgccctggaatcacgtagaacttttttaaaaaatgcatcagaaaaaaagaagggctgcgctgtgtcacctgagatctgtgctgtcgaggcttgtggaagtgttcattgcatttgggcatttcgcattctgttgtgaccacaggtggttcatcagagtgtcgcacagaactccactcaaaaggtgctttccttcaccaccacgaccctggacgacatcctgaaatccttctctgacgtcagtgtcatccgcgtggccagcggctacttactcatggtaacgctcgatgccatgctcctgggggctggagtttggtttggttgttttagtctttacttttccatgactgctcctgcttcttaactgctcttaacatcgatgtgcatccaggacagagagagcgtggcttcacagtggtttaaaagtggtcgtgtaaagcatttttcccatttgccgtttgcctacctttgactcctgtcattttatgtgacgcccttgatgtaaagttttgttgttgttggataagttctgccttttgatttggggtgatgggtggagggaaacattagaatcacaacacattctaatgttcggcttttgttctgtgcccccattgttctgcttgcagctcgcctatgcctgtctaaccatgctgcgctgggactgctccaagtcccagggtgccgtggggctggctggcgtcctgctggttgcactgtcagtggctgcaggactgggcctgtgctcattgatcggaatttcctttaacgctgcaacaactcaggtactaaaggagccatttatctgctgtccgttgacaaatgccacccggcccccaggcttggcccagccagcctcctgtgtgctgtgtgtccttctcaccggaagggtcttgcagccttttccactaggatgctactggtttcaggggaatgctgtctcttgatgctgtccgccctcagggaagtgggtaggaagtctgttgggcttattggttagtagagacacttggctgataggaatgtatgtcattgaccagggttgtggctcttcagctctggtgggcatttgcatggcttgcagggcttgtgaaagctccgattgccaggccccacccccagagtttctcagtaagtctggggccggggctgagaatgtgcacttcagggcaattcccagtgatgctggtccatgggccacactctgccaatcattgcttaaatgagttggattccttctcatttactagcttaaaaaatgatagtacactgactttcgaagtagaaacgtgcaggatctcctgtgagctgaaatggctaggctttgggacgtcaagtgaattcttcaccatgaggttacggaagccctgcttcaggagctgttaggtgctggtggcagagtcctaactagctttagaatcatctgaattgcatctcgcatgtctaatgccaccatcctctgtttttgctgtaggttttgccatttctcgctcttggtgttggtgtggatgatgtttttcttctggcccacgccttcagtgaaacaggacagaataaaagaatcccttttgaggtaatgcaaaaacaaaagaagagagctttggggacatcacagcttcctctgttcctaagtatttgtcctttaattttcctcagtctgacccagtaaagatttcagtcccatgtcagatgatgtgtcactggctgccagtcaatgaaataagctggaagactctcaggacatgtatttgctatgagaagtcccaagagctgcatctcgggtgcctttaggaatacgggtgtttgttttaatgtgacactttatccctaactgccatacattcccagagcctgcatttgcttctctggagatgtctgtcacttagccatcacctccctccactcccttttaaaattatacttaagcgttaagtcattatcttgcagctttcttctctggatggtaataaccatgaagaattgatgactctgtagatggtactgaataatgctggtgttagggggtaaggcaggtggctctgtttccctaatgccagcatgataagctgtaataacagttgtgttattctgccacgtatctgctcacacagtctgtgctccaaggggaccatgtccagtgcagctctcagcgctgtgtttttttattcccaggacaggaccggggagtgcctgaagcgcacaggagccagcgtggccctcacgtccatcagcaatgtcacagccttcttcatggccgcgttaatcccaattcccgctctgcgggcgttctccctccaggtgagcttctggtgatgaaggctgtggtgatcctgaacggggcactgacttccagcatcccatgtctgcggtctctgctaaggcacttcagttacttatgaagtgaaatagaaatgcctgtgtttgaggctctgtttaactgggtggatgctgctttaagcttataaccctgacactccctctaaaaaactaagcccttctggagtttttccttcctttctagccctatgaagcccagacgtctgttgccttatagagtggccatgtcctttatgcatggctttcggggctactccatgttgccctctggggtcaccagcgctgcagccagcttctgggccatcccctgcttccaaacaccactcccagtacctgcacttaccccctctcccgctgcccacttgatcccaaacaggctaccttttctttcgcttttgccaaagcacccccaacccaagacagtattaagctgaggtcgcatgtcagacggcactgcaggaccgcgaactggcgaggctctgtggagaatttcctgcagggtccgcctcggtgggccctttctttccacctccctttcctaagttggccgccacaatctttcctgtcccctcttctctttccttttggtcccctgctcccctctcaggctgcccaagcagactcatgatccctctcaccttcccatgcagagcacgtgggcccctcgtgacttgtcctctccttccagaccaggccccgtcacccccagaggccgtcagggtattttctaatccagactcagcttctgtgaattgtaagcatggacagttccctgggatggggatagagctgtagtccgttctgcgcctggagtaaattactgacgttgcccttgagctacctgaaattcataggaatcctaaaattagttactgcagccagtaaagcagagacttcaccagtttgtgccatggtcttcctgagcgcaaagtggcctggagttttgatatctcaagatgttgtgggggttttgtgaattaatttcaaatttcaaaacctcaattgtttcccctttctttctctgtgatgatatgatgtctaattctcaagctggccaggttgttaaaggaaatgacttctgtgtcactttttcacttgttgttaggatgcccggcacactcacacagctttactctactcaccaagagttcctgcagatgcgagcaggaagagaaattgaccagtcttgcatctggtttctgtttatattttgtcaatatgtttagctctttcctagtctttccatttgtcatgtaagaggaaccaatcaatcacgtatttgaatgtcatttcgctagtaaccaagaggcctgggcaactactgatcctactgggtttgggaatggaggcatctttagtatttagtgtgaggcacagctggagaaaagattctatgccaattaccgtaaccgccagccccaaaagaaacataaaaagaaaagaaaacagggtttatttatattataaatttatatagaagtaaaaatagtgaatcaatgaataacgttcagatctgatgctcgaaccaaagcacaggacaaggaaactctctctgattgctcagtggcatgaaaggtgaaccagactcgcagtgcagctgaagttgtagaatgcctttgtgcgcaggtgtgtgtctgctcagcaccctgtggtaccccagtctcatttcacgtgcagccggtgctggtattgagaaactgttctagagtcttttagtcatgggaccctgtctgcccattctctctcctctccccttcttttctgcttgcctttttgctcactgtctttctgtatctgctcatactccttcttttcttcttttctctctcattctctcctctcctctcttagtacatactgatggctcatcgaggccgactctcctttaatgataccctgtggtgtggtggtctgaaaagctacatgaggtttccttacgaggagtagccttgtagaaaatccagtgcgctaaattctcccaaggtaagcaccttacagagacaaaaatgagtactgttggtttttgtgtttctgtttcaaagcaccacagtatcactgtagaaaattgcaactatagcttccaagagtcctcatcttactgaagaatacctcctcatgacactggttaattctctgtcaaaagaaaaaaggagtgcttcgcacggtggctcacgcctgtaatcccagcactttgggaggccgagacgggtggatcacgaggtcaggagatcgagaccatcctggccaacatggtgaaaccccgtctctactaaaaatacaaaatttgccgggcgcggtggctcacgcctgtaatcccagctactcgggaggctgaggcaggagaatcgcttgaacccgggaggcggaggttgcggtgagctgagatcgtgctattgcactccagcctgggtgacagaacaagactctgtctcaaaaaaaaaaaaaaaaaaaataggatagaaaatgcaaagaaaagagagagtgtgcaatgatttttgcagtggccatcagaaaattttccctggttctcttttaaatgtattgaccctcaaaattggccattatgtagcttctctttccattgatgaacagtatcaggtttgactctgggtgttcagttgtgtttatatttgcaagatgctacagattaaacatcaaaagggagtgacccaaggcggttctgtccaatagtaaacctgcgttaccttcagaattttccatataaaactcctccaattttgtagcctgaccaaataattagatatttttttccacaaggaaactgtcttaaacctcatcagcctttatccatctgcctaaatgccatctctacaccagaaatcactggcatcagctgtaattctttgtaattaactagggcttgctggtagcccagtagtgtttattcttcaatgaaaactcacagaacagaaccccttgttgttctgtctggtctaggagggttcaagtggaacctcctctgcatgtgtcccagccttagctgctttgaggggatggggcatttcctatgagtacaataactagaattgattcacagttgcagtatcatccccatcatcttctctggtttacttacactctcttgcctttgcgacaacccttcttgctttctgagcagcccttgctggttgttttactattttggtgtacacctggggacctggcactgagaaaatactccgccatacatgtagtaggttatgagaaagtcagtaagatgggacgctgcttggggttcttccctcatcccttcatttccgtcatggacacggagcaaggtgcccagcaggcttctctgaccgatgaggccggctggtgcccatgcctgtgcccgtggggctccctgtgtcacgaacctctgctctcctcatcctcatgaacacctgtccaaattgggatgcccagggtctcttctctacattccactcctccattctaaacatgactgattttaaaagctaaaaggattcggcccccaggatgacatctggaaaatctccgttagaatcctacctgttcttcctgccatcccctgctgtctcacggtcaggtgtcctaaccccgtccacagacgtcctctcagtaagggaccagatctcacaaaactcccggttctcagctgagctaggggcttggcatcaagtctttgtttgatctgatttactggataacctcaggcaggtgacttgaccgcacttcggggactttatcaggtaatactgtcaagagcagtgagaacctcctgtttgctatgaatcaggtatgaacgatctgatcttcatagcttctcctttctcttccccaaagtgaatttcaaaccccagcagtactgaggtgcttgagccaggtgtctcctcttgcacactctgtgtttttttcacagggagcacctgccattcccctgctcctgactgatctcttttgcttttcttttttgagacggagtctcactcttgttgcccaggctggagtgcaatggtgcgatctcggctcaccacaacttctgcctcctgggttcaagcagttctcctgcctcagcctcccgagtagctgggattacaggcatgcgccaccacgcctggctaactttgtgtttttagtagagatgggtttctccatgttggtcaggctggtctcgaactccaacctcaggtgatctgcccgcctcggcctcccacagtgctgggattacaggcatgagccactgtgcccggccctctttttcatcttataaaccctgctgaagcctctcccgggaagcctgccttgactgaccacccctccaccccactgagctgctctgcagcctgacacctgccttcatgacgccatccatcattcgagattgtcatttgtgtataggtctggctctgtgagaattgagagtaggattttgtcttatttatctttgtgtctccagcacccagcacagtactagacttttctttttaaaagatctctctctttctcaggtaaattctcttgtcacagtggtgatgagcttatttgtgtaggtctatcagacaggtcttaaatacattagagtggtaagctaacatgcttcatatttttcagttgatgaaggattttcaagatagaccaagaagaaagaggttaggtagtttttagaagaacgctcttgagagggaattaattattggaagacatctctccggaactcaggcttgctggttcaccttcagcataaactcacaattcccatccaaagccctttgtggaagttctccctggattgtgttaggtggcagttatagccaggttttctttcctcaccctaggacgtttatcagtttgagatttgtgaactaagcagttctttgagaagcctcgttttttattttctttctttctttctttcttttttttttttgagacagactctcactctgtcacccaggctggagtgcagtggtgcaatcttggctcactgcaacctccatctcccaggttcaagcaattctcctgcctcagcctcccaagtagctgggactacaggcatgtgccaccacactcaactaattttttgtgtgtttttagtagagacggggtttcaccgtgttagccaggatagtcttgatcaggagatccctgaccccgtgatccacccacctcagcctcgcaaagtgctgggattataggcgtgagccaccgcgcctggcctcgttttttattttcaaagccatgtctggcaatttcctggctcctcagcattgccagtgcttttctctgtggtgccccttctgccgtgcccttccgagctgggtcgacttccaccggcttctgttggcttcccctattaacagcccaattttgtaatttttactaggtcagcagaagatttgccaacgcactgcagaatttagccctgtgtttattctaggttatggagtgacttttgaatctgtacttatttattctgcgtactataatcacacacaggaatttctttctgcttaactctccttgaccttttatgtttcttgctctcacaccccacaggctactccagaggcaatttggaatgtgcctattaaaacctcaggggacacgtgtgaaattgtatttcacgttttaaagggggacattttgatatactcgatccactttatataaaaatgtttaactgtaaaatcagggtcgcgtgacctcttggaagcattctggaagggaactgcttctgtttgttttaaattctatctcgagtgtgctctctgttctgcctttgtggatttggtttggttaggttactagcaacacgttagtagtttagttcccaggtttgtgccttatcggtttatggaattattagagatagaaaggcccagaaacagtcaagactattcctatcaaataggctcttgatgatgtagcaaaagaaactcttaacatctgtcacggtttcaaatgcttcaagaggaaagggaaagaaaaatattgtttaaaagcatctttattttcgaagcagtcctctgattgggcggaggcatgttggtgacctctgaattttttttctgctcccaggcagcggtagtagtggtgttcaattttgccatggttctgctcatttttcctgcaattctcagcatggatttatatcgacgcgaggacaggagactggatattttctgctgttttacaaggtacattttcagactgctgtggccttttgattgggtgcagaatggtgctgtgagagaactctttcagcctgtggacttataaaggaatgtgggtaacggggtttgtgctggtgtggagaacgtaggggagagattggacagaggctgcttccctagggggacatcagagtccctgggcttccttctcccctgccgcagctgaacagtctcacagccctcaagatgctggttccgatgacccctttttccatcatgaactgatgttgccgattgtaggaatgtttcatatctctgcattagagcagaacaagattgaaaaacacccatctagaaaatatatagtcctttgatggtggtttggttttgtttggaattgtgctttgaaggattcatcatcatggaacttgcttttgaaagccaaatttaggatcagtcctccatgtggcctcctcagactgacttttatctagttcaccaggcgatgaaccaggtgatgttatcaaccaggcgatgtttctggaattcagcaatgtgatgtttctctcctaagtcagagctgtgtaaaatgggtattctccgtacacagtgaaaaatggcagaatgaaagcaccatttccctgtttcagcataacattgcaacatgtttcccctcctttcagcccctgcgtcagcagagtgattcaggttgaacctcaggcctacaccgacacacacgacaatacccgctacagccccccacctccctacagcagccacagctttgcccatgaaacgcagattaccatgcagtccactgtccagctccgcacggagtacgacccccacacgcacgtgtactacaccaccgctgagccgcgctccgagatctctgtgcagcccgtcaccgtgacacaggacaccctcagctgccagagcccagagagcaccagctccacaagggacctgctctcccagttctccgactccagcctccactgcctcgagcccccctgtacgaagtggacactctcatcttttgctgagaagcactatgctcctttcctcttgaaaccaaaagccaaggtaatctgccacaacttaaggctcttcctgtcttcaaaaaaataacagaacctttggagttcatcagattgcttctacttttcttctttttccttaaaaaaaaaaaaatttcgtttaagtatgatacatttcagtcctccaaaagtcaatgcttttagagcatagggccattttaatgaggtcatcttccatgggagtgaacctctatgcagcaagtctttttatgaatgaggttgtttaaaaatgtgattattgccacttgcggtggctcatgcctgttaacccagcgctttgggaggctgaggtaggaggattgtttgagccaggagttcgagaacagcctaggcaacaaaggccaaccctgtctctacaaaatattttaaaaattagccagggacaggtgtggtggctcacgcctgtaatcccagcactctgggaggccaaggccagcggattgcttgaacccaggagttcgagaacagcccaggcaacatgatgaaactccatctctacaaaaaattgcctgggcatggtgatgtgcctatagtcccagctgcttaggaggctgaggtgggagggtcactgagcccagggagtcgaggctacagtgagccgtgatggtgccacttcattcaagcctgggtgacagagtaagaccctgtctcaaaagagagggggaaaaaaggcatggtgctgtgtacctgtagtcctagctacttgggcagctgagacaggaggatcatttgagcccaggagttggaggcaacagtgagccctgatcatgccactgcactccatcctgggagacagagtgagatcccaactcagatacataacaaaacaaggctgggcatggtggctcacgcctgtaatcccagcacttggggaggccgaggtgggtggatcacctgaggtcaggagttcaagaccagcctggccaacatggtgaaaccctgtctctactaaaaatacaaaaaaaaatagccaggcgtggtggtgcacacctgtaatcccagctacttgggaggctgaggcacgagaattgcttgaacctgggaggtggaggttacagtgagccgagatggcaccactgcactccagccttggcaacacaagactgtctcaaaaaattatatataaaacaaaacaaaataaaaatgcgattactatgattttatcctttttcctatgggagaacaacccctacaagataaatcagtttaagtgtggtggtgaaaacaaggtattaactagacagcttctctttgtccaggaagagtcagtggtgctccctggggtctgaccttgtgcctcttctgttccaggtagtggtgatcttcctttttctgggcttgctgggggtcagcctttatggcaccacccgagtgagagacgggctggaccttacggacattgtacctcgggaaaccagagaatatgactttattgctgcacaattcaaatacttttctttctacaacatgtatatagtcacccagaaagcagactacccgaatatccagcacttactttacgacctacacaggagtttcagtaacgtgaagtatgtcatgttggaagaaaacaaacagcttcccaaaatgtggctgcactacttcagagactggcttcagggtaagacggcgtgggaggctctcgtcctttcggtcttttgcgtgttcagcttcaacaggttcctttgtcatgattatgagagcgttagattagaatgtaacactgacgtgtttcatggacttttaagagttgctcaagattcttacatgaactgtttctgcagcagcctcccagatctggcttttacctataaaatgatgcagataattcagatgagcttctgtgagaatgtacaagtgcacgtcaggaaaaggggagcatgttgtaacaggtggagccgaggcttctggtgctctttcctcttggatagacggtggctagtttggtgaccacataaaatcaaagtgcggctgggatttccagactgattgccaaattatagcttactgcaccaggagatgctagataagcagtggacatagtcttacactccatattttgaccatctctttaaaaattatctatttctaattgactttgtttttagagcagttttaggttcccaggaaaattgagcagaaggtgctgagatggcccatatcccccttcctcacccacacacagccccctcgactgtaaacatcccgcaccagagggatgcatttgttacagctgatgaacctgcacattattacccaggctccatagtttacggttgggttcattttgaccattttttgatttggtacttgaagtaaagttataaatgcttagtaacatttcacattattttataaataattaaattctgttttaaaatgcctttcttggtggggcacagtggctcacgcctgtaatcccagcactttgggatgccaaggcgggcagatcacctgaggtcaggagttcgagaccagcctggcctactagtgaaaccagctctctactaaaaatacaaaaattagctgggcgtgctggtgggtgcctgtaattccagctactcaggaaactgaggcaggagaatcgcttgaacctgggaggtggaggttgcggtgagcctagattgtgccattgcattccagcctgggcgacaagagcgagactccatctccaaaaataataaataaataacagtgcctttattggcttcttcactcttagaaatgtttctaggaaatattcatcagctggaggggaaaaaaaagtctaaattttctcctgaccgactgcacgtaaatgtatgcagtgattccaggaacatctgttcatttattctgcattgacacatgcaaagccaagtagagaatcaggataattatccacccaattcaagcaaaacacttttactggagtaccaaatcattatggcccccaacattttttggcttggattatccaaatgattgtcttaatttcccacttaattaggcttgtttaaacacagatgtaagtgccttttttggtctgaagtcaatacacttgtatgtaaagactcagcagccagtttccctgagagtttgcaggaaatggtcaacaacttgaggttggtagaggatggttccatacagcttctggccttgacttgtgtttaccagaactttgtgtctacatgtacacccaatttgactttccttttggaggaaggtacagagaacatgccatgattattatttattaacaccctcctgagacgaacgtgaatttgttccaagttttatttttcacatttagtttagaaagcaatggtttttgggataatgagttaaaagagagacagtggagagagtccctgctttcattttccccccattgaccaaagacatataaataacttgccacaaatgacaaagagatctgctgtgccctgccagtctgtgagcagagaagggttattgtagagtgtatttcagtgtctttggccattgctctggaggctcaatgtggcatgttaggcagggattgggaagggggtaaagggggattgtctcgaggcagtgcaaagggttaacctttttatagagcattgtagatcatctctatacgtttcttcctttatagatgtaaatcagctggagttgagtttttttttttaaggtgcatttgaatcatgttttgattgctgtgtgaacagcatacacctagaccaagtgagatccattggtgctaagcaatcagggtcccaggcagccctgggactccatgactgactcttacagggtccgctagagcactcaacacagaatgaccagacactggagccctttgctgcctacacgtttttcaggcctgttgaatggttgatgtggtgtttgaacattttctacaaggaaacaattatccttgagtcagctttccttgctgacatttttgtgtggcttttaaatggtgggagcctcattggtttttaatggaattcccattaaaactatgtcatctcattggtctttatcgacattacaatagtttgtctttgattttttttactcctaaaactatataatggttaatttcaaaactatcccacatgtcacatcttaaaacatacatgcaaataaggcagagtcagataaaaaccatgcctcgagtcatgtacaaatgtgttcatttttatttcacactggattatgcaaacaaaattttgattaacattttacttctagggtcccatagctgaaggaaaatgggcaattggggttattcctttggcctagaactttacattagaaaataattattgacacaaaatattcataagaggagaacttctagctaagatattaaggtttgctgtaaaataagtaaaaagtcacaattttctagagtgtgcatgcattggaatatgaagcactggctaaagggcagtggaaacccaaccttatcccttgttttgtatccagggtcgtaccttgtacatgtgcttaggccagccccttgttacttgagctttttggtgattagtcagaaaatactgtagtaatggggaaagaacttaactgttccagaaagtcgatgactgtttcgtattcatctttgaacccccagtagagtgaatggcacctaagtgaccagcagtgtggattctgagttgaatcgagtttccagcaaggctgacctccccagataggatctttcttcctttgtttcaccctcgggtgtcccagaagtctttgtgtgtgaatatcataaaaccgcccccagattttggggaccccgtttgccaccagatgttattgatgctggagttgaacctgcttgtcttactcctgcctttgacacatccttgggtaccacggtgtgtccccgcactgtaggccagtgagtggctccagctgcctctggtgacttgctgcctgggtttgagctccatccaacactcacctgctgggtggccccctccagtctcccctttccaagactcagttcctccatctgtgaagccggagtgattaactctaacggggctgtcccttggttgccgtgggatgcccagcgccaccccacactcaccaagtgtgagcctctcttcaggattagctggttaagggagaggggcttaaagcccaggacacgtcatgacatcatgaaaattgttacagcgaggaaaacagacgaggcctccctttttgaggggaaaggaggcgcagtaatttttcttctgctgtttattgtttttcaaaaacagaaagaaagggctttcagtaataaaataacatctgctcagtaccctgtgtgtgcatgcctccctatgaaaacccgtctgtctgagaggtgcacggcccacccggtggtctcctgtcccgtctcccctccacgctcaccctgacacagactcctttcaccaccgagcccactgtcatccccaaattgttcctctagccagtagtgtcagaaggagggtttcgcttcttagagagacagaagggggcctgtgctccccctccctccccacacttcagaccccaagagcttaaccattctgagcactaacgcaaccagattttttttgaagcctttgaaatcatccaaggcatgtcccgaaatgaaataaacgcttctcccttcttactagccgtgtccaacccatacatgtacagcttgttcaaaagctgtggtttcctgagctgctggggcttgggagctctacttgggaggtggctgggaggggcaagggtgggtggggggaggggggtgtcccggagagcgtgcgcaccagccagcgctcagaccctaaggccgggtcaccggggccctgttttctcttccaacatgcttatctctttacagctgcagccaaaccgtcaggtctgtaatgatggttttgattactgaggccaagcgcagctgtgtgtggtgtgctggcgggctcgcccatcccccaggcccagaaagctctttcctccccctccccggcttgtggtttgtcatctgttctcacagagcctccttgtattggcctgtggcttcctgttctcaatcggatccaaatttaccctgtgaggtatttccctgcgtgcacacgagcgccttcccttccttctcctccaccccctctcctgaggaagggtggcagagaggaagagagcagtcgagagtgaataggtgttggctgtgatctttacacaacctggcagcggatcccacagcttgtgttagctccacaagtcgaacagatggaacaaattgacagaatcgtgttcgctcaccctgggctccagaagcagctgagctgctgtttgaagttgggggagtttgaatgtgctttttcccctctgtgtggaatttctggttgtattgaagaactcatgacttagcaccccgagtggcacgaaacggttaatgaccctggtgcgtgtctttccgtcgcactcttgccttcttttgtaattgtcatggggcgaaaggaggagagtggtttttcttaagataaaatatgagtatttgtttcatagaaacagataatctccttccccccttttgagcagtggctggcagcagctgcagttcattcatgaaagatccagtgctttcagtcaaagtggatgtgggacacagagggtgtgtttctgatggacgtctaagagccccctgccctgctcagtctcctcagcccaccccgctaggaccagggtccttctggctgcgagttataatgtgttacaatcatttgccatttctaggacttcaggatgcatttgacagtgactgggaaaccgggaaaatcatgccaaacaattacaagaatggatcagacgatggagtccttgcctacaaactcctggtgcaaaccggcagccgcgataagcccatcgacatcagccaggtactccagctgctgggcactgagggcgcttccagggctgcggggccgctgccctggtggaggctgcttgacagcgtgtgaccccaccctgtgctctctggagacctaaggcactgggagctggtagaaagaatattgggtgactctaattcacctcctcccaccagctaattgtgtgacctcaggccagccagtgcacctcactttcctcatctgtcaaatgggacaggcgtgttcctaccaccagaacaattgtcagaataaagtgcagccaagagagtgcaggctctttccaatgttagaaatgcagcggcgtgtgattctttcagttctaggactcggtaccttgttggtcttgatggtattcaagcttggaaagatggatttcttgggtagcagtcatttggatgagcagaatttggggtaggtgggtgggttaggagattggagaggtgacagaaggatgggaagatttgttacatttgagatctttgggtggccctggccccaggagtagacagatcgctctgtgatgagtgaagcaaacggaaatgtgcacccttggaatcagagtggctgcgccattctgctcactcctgcagtaataaccgtttctgtttgggaatcatattctccacttgtgtttgcatcttcttgctgccatttggattccagcataccccttttggttctgcaggtgtgttcaggagtgtgttttggactcctccaattgcacggggcccgggctacctcagcttcctcggagcctcttcccttggctctttgtagcttgcttgcttcaccttcaggcagaaaaccgtcatgctaatgatcttctcagcagctatgtttattgtctccccttttttgctggcattcaaggtgacagatttacaaagtacattgttcagagttgttatggagtgtgaaactctgagctcacagcagcaggcagcagaccatcacgtttcccaaggatgcaggcagaggagtctgcctcagagacaccaggaaagtgcccaggcagcagggctgggcgagggaccagggatactgtgggacaaatgggattgggccagacccccagataccatttcctcctcccgggagggagaaaggcgtgctgcatgcacagaggccacgggctgcgctgtttgattccagggatgtgttgaggacatgcagttgttacatatcctgtggattaattgaatccagatccccgaaatccagttctctcactctctccctgctccttcttccttcactccttctctaggagccgctgtcactggtgtttttcattttgtttaaaaaagacccaaagcgaggaatgaggcctgaatctaaaataaacaggaaacggaatttttctatgtatataaaacgtacaaacacatactgagtcccacagagttccccacctcagcaaagcccacaagccctcctgccgagggagcacatttggccacgcagaggacgcggcgtgcggagtactacgacctccgggaataagatcacctggcgaggcttgagggtgtgcactcgtcccttgcactttctggcgctgggcattttttgaatccacaggtcataggaggctagtgttagcaaaatttatacagatgttgttttatttttatagctctcctccatttactgggtggcatttccttcagtgttaacaacagaggttaactgcacacagacgtgcatggggattcttgccaggtttctcccccttcccctcctataaatatgtttctccagattgttcattctctgccttggctgcagacggtcttttagcattcagatttaattggttcagccaattcctgggccatctgcagaactgtgtttgttataaacacgccagtgattgcatcctccgataagtgcacattgaaagaaatcaaaggtgttctgatgggattttcgacactttcaacagtaggcattctattctttgttgtaatgctgtgcgaagctcagcttctgtgctctcaaggcagaagtgtgtttacccccaaccccattctcaaaggcctctgttcttcccgtttttgtagttgactaaacagcgtctggtggatgcagatggcatcattaatcccagcgctttctacatctacctgacggcttgggtcagcaacgaccccgtcgcgtatgctgcctcccaggccaacatccggccacaccgaccagaatgggtccacgacaaagccgactacatgcctgaaacaaggctgagaagtaagtagcgttttatcgggaggtctgggaacttacagaggtgccttccctggttctccaaactcagcaacagccttcatccgttgacgctacccagggttcagaagtgtaaacagaacaatctatttcttctcgtttaaaactctgggagacgaggatgtgtaatgggatacaacccccgcactctatcacattgggaaatcagtgtctcgtctccattcgtagctttaatgctaagacgttcattaggcgttgctctcacgtgccagctgttccctgtgaacaccggggacgcctccaggttaagtgttattttactattgcagagttttatcgctggggattatatgtttatcttaactttgtcaactctggtgttgaagggatttttggcacattgggtcattcctataagccaccaagcacctgcactgtgtcaaagctgtttaaaccggagcagaaaagtctgtatacaacagcgtcctcttcctttcacctctcagcccagagtttgaaaggtggttctgtgtggatttcatttgcatttctggtggagatattagtagcgtatgctttggataccgggccaagggaggatgtgagaaagaagacgggtccccttgcaaacatggctggagactcagcgagtagtgcttccccggatccagtgctgcccagagccctgtggcaggcagccaaggcttgtcttaatgaaaatttatagttgagattataatagggcaaatcatgtttagcaagtcaactgaatgaccccagagggtctggaacacacaacggcagtaactaaatgcttgttgtgtaaggatttcttcccctgttgctgaagacttagcattgcattagaaaagaagcatacaaggttgaagtttgttcaggcactaaaaggataagttaagcatttttctcaggaaagcatgactcttgagttcttttgctcttgcaagtgattgaataaaactgtgtgaacttggtattttcatatgtgtcaagggaacaaaaaaatgttcccaaaatgtaagcctggagtttttagacccctcacaaagaatgactgctggaagattaaaatcaaatcttaaaaaatacacacatcacagagttaaacatttttaaaccaaatgggaagatcgtaaaggcctggaggctatgatcagcattgtttgttctttgtatcatgtagaagagcagtcagtaagctttcatgcaaagttcttctctctttccattgaaactgtgatgctcttctaccctgggctgctcctaacctgtgcccttctctgtccagtcccggcagcagagcccatcgagtatgcccagttccctttctacctcaacggcttgcgggacacctcagactttgtggaggcaattgaaaaagtaaggaccatctgcagcaactatacgagcctggggctgtccagttaccccaacggctaccccttcctcttctgggagcagtacatcggcctccgccactggctgctgctgttcatcagcgtggtgttggcctgcacattcctcgtgtgcgctgtcttccttctgaacccctggacggccgggatcattgtgagtgtattataaggggctttgtggaagtcaaattcctttcagcatagctctttctgcagccgggaagttttgtttatgtctgggcctctggaggagggtatagcttgcatcatccgtaatatgtttattctactcgaggtggtgaggtccatggttaaggtagaacctctaatggccttattcgtaggtcctctctcctgtagcttgaagatttttgtggaaagataggggactttttaaagcacaccttccatcacagctctgaacatttattaaatcaaaacactcaaagcctagctcattttttctttaattgaaaagtgtcattttgtgacctcatccaggcctaataacgacatagcaggttcttctgcatttaacatgaagggacaatcagagaagttaggagagtgatatatggagaaaatgattttgtgtgttttaagtcctgggggaaaatgataaactaatattacgaacctgttttttggcatcccttctccaaaatactctcagattttgatgtaagtttcatgtagagtctaataaaaccgtggcaatgtttaaattctgtgaactatggtatagcctttaaaatgccaaaattataaaaacaaaggccagattttgtgtctgcaccgtgagtctgtgcagcaggtacattaaaaggtaaatgtttagtctgtgagagttttcatggcacttagcaattgatgttgtccatctttcatcattccttcccctccctccacctctgcccaccacccacccccagagaagaaagatcatttttctccttctcttccatctggtaacttgttgtttatttgtatcttctccgctttggaccaagaaactccaaatctaactcacagattttatttccatttccccacaaatttttacaaggaagatgaaaggcaagaagcttttccttggattttaacagttggctcagtccaccccagctgtccccagctcttcaaagtgcagggctcctgtaacctgacagcgtcagctgatgacgacctccccgcttacctggcccccacatcatctttgcttagcctgcaggttcactcacaaaagcaaaactactttgacctgcaaatcttttattcaccccgtcctatcagctttctttagaatggagagtgaggcaaccaaaaacttaatgatgggttcacggatggtctctggcggctgagacccaagcaggaggagagatgggtcttgttgtaaactgtgaaattcgcagctaagtgatgcggccatgcttgtgcaattcctcgtggattgcgctggtggagttgttctgtatgttttttgctttctggagaaggtacattggaaaataatttgcattagaacactctactttgccttctacagctttacacatcagctgtcccacagtctcccttcccagcgggaagatgaattaggcagtaaaggcagtgtccagagttaaagcaagtcagtccattcattgttttgatctgaaccgaggacaccttagccctctggcgacctcccttgtgggcagccccagagggtgacctgcccgcccactgaccactgtgtgccctgctccaggtgatggtcctggcgctgatgacggtcgagctgttcggcatgatgggcctcatcggaatcaagctcagtgccgtgcccgtggtcatcctgatcgcttctgttggcataggagtggagttcaccgttcacgttgctttggtatgggagacatttgaagcaaaaactttgtgaaaaaacaacccatttcctctggctctgtttgtctccaagtgggaaccttgcattctccctgtgcgtggcagtgaaaaggcctcagggcacaggggccccctaggagagcctgcaagatttctggattcctttccctcaagggaggctaaggagaggggtatggaaggtacatggcagcgggccctgtcagccgtaggcagtgcagggttggtgtgttggagtgggccttgaagggccgtgctcacctgactttgactgtcattatacccccacgattcctgacgttgtgacgtttgttcataaggcggattatgtggtgagtgtgtgggtgggcatacacagagttttaaaaacaacaggaacagtggggataaaatgagaccacactcacaggcttggggatggcatcactgaggatgaggtgagctggcagccaaggaagtaccctcgggcttaggagtggaggctccattgttcagccccggggaactggctcttctacctaagcccacccagttcctcagacctcccagcctctacattcatgatagcagaaagtaagagaaccagtgttgctttcaacagacatttttttccctctctttttctctatcttgccctgtgaatgctttgttggaggtaattaaacttcagaaggaatctttagtccaagagataaatctgttgtatacatttatcaaagaattatccaaaacatcacaactgtgtaaattatgttctgtctgcagattagagcctaagaacttggggttatcagtaggcattcaataagtaaatgcgttcacctgttaaatatcgacgtgcgaaataccctagctaggaagccagcactgggcttgaccaaatcacttccgccattgcttttgcaatctctagcacaggtttcctttgggctcagcacatggcacatgcatgtctccacataggctgcagccttgtgccttctgcagtctttccagggctactgtgatacaattgagagattgtaaacttttgtgttttggaaggaatgctttgttgaagttatcccattttttttacagtgtatcataaaaagccaataatatggcaacagtattctctaaattttgagcctgctcaaattattttcttcctttgagctgggaggagggactatttttttaaaaagaagagtgccagcctagagtttagaaaacccataattccaatacagttcttcaaaagggagataaatttgcatgtgagttaagaatccataagttattttattctgatgcaaatggaaaccaccttttttggaagctagcagggcaaatgggtatcccagagcagatttttaccatggaaatgctaactgcagtctctaaactctaggacttctagtccaagtcattttcagtattctgatcctctatgtgaaattgcatttagagttggcagttattgctctttcaaggtttgtaaaatggtagaataaggctgtgaagaagagtcttctttcatgaatgtctttaggaaatcagtgggttcccgttcatttgctgtctcagtaacatagaaacagtgtgtgacagggtagtcacaactaggcccttatggcctcatggcccaaatatgatgcaagccagcatggctgaaaaacccctttggtgccttgccccaagagcagaaggcagctatattgaccctaatgtctttaccaactctgaaagagtagaacctgtttgagagaaaagtatgaaaactcttagcacacctttgtatatttgtattttttcatctgttaaggagaacgaccgtctcagtccttctgcaggtaggtaagggatagaaacgcatcctgccaaagacacccagccctctcctctcggcataatccagagcagcctgaacttcagagagagaacttgtgccaggttcaacactccctgcaggggtcacacgttcaggccaacagtctgatcttccagaatttactctgcaaagttcccgtttagttatcaacagcatttctggcctaggagagctttaggactgcaggagagacagggatgtgtccacacaatgttcagaagggcttgattcaaatgtacggtcctgtgtatttcttgcaatacactcatagtgcttagatgtactacaaacatagcctgaaacagtgacagaaggtctcacctctgcataatcgtgaggaagtgtgtgtagcacccaattgcttgggacactgagctaagctcttagccatccaatgcattagccagaatcctgtgctgtgggaattggaggtctcacgtgtgagtggtgtgagatcatttaaggaggggggaaacttcggggtgagtatcagtgaaaagaaggggacatttctgtccatttttatatagttttgattttactgtgttcaagatttacgtcaacaccaaatatgacccagtgtcatgaagaggtcatcttagtttaagtctaggtgctgctagttgagctactctgattacacattctagcaactaaaacagtaagatgtgagcagttctgagagcttgtaacatttaggacagagctgagcatttaccaggtgaagtccagcaacctgatcttgtgaacatcctcattgcacaggcctttctgacggccatcggcgacaagaaccgcagggctgtgcttgccctggagcacatgtttgcacccgtcctggatggcgccgtgtccactctgctgggagtgctgatgctggcgggatctgagttcgacttcattgtcaggtaagcaggcgtgtgcaaggagacatgttttagaaatcattgtgattgggccgggtgcagtggctcacgcctgtaatcccagcactttaggaggccgaggcgggtggatcagaaggtcaaggagttcaagaccagcctggccaagatggtgaaactcatctccactcaaaatacaaaaattagccgggcgtggtggtgggtgcctgtaatcccagctacttgggaggctgaggcagagaattgctcgaacccgggaggcggaggttgcagtgagctgagattgcgccactacactccagcctgggtgacagagcgagattctgtctcaaaaaaaaaaatattgttattagctcaggtgatcttcgttgcttatttccttctgtatctggtagattgcagcacctcgtcatcgtgactctgctttcttttgaacttgttaacgtagagatattcggaggtataaagacaagctctcttctagagctgtcttggggagtatcttgaaaacagaaggtaaaggatttggggctggcagcaaagacaactctgcagcttgaagccaaaggatcacccaataggtggcccattattgtctcgggttagaaacactgcagaaagaaatgccacatttgacctacgctccctctccttcaaactgtagaaaggaaaacttaaaaatacctaaaattgtcacctgactttattcttttaagaagtgaaaatgacgtgctcagatttcatattcgagtccccacaattaggaagcatttagcattggactttggtgtcaactcttgggaatctaagcctagtttggatctcaaggcaagtcgtttccagttgatttaattaagaatatgactttgtgaaatataaaagaacagtgagttttccttttgaaaagaatttgagaactgcagaggaggaaaccacttgcttctttgagcagcctccatggctttgtgagggacctgctcttggatagaactctgctactttatggagctctaaggccagaatcaccaataagtaaaagatagtccaggattagaaccatgacagggcagaaccagatgtgagaaatgattggtttgtttggcacgcttcagtactgagaggttgtgtgtgtgtgtgtgtgtgtgtgtgtgtcagagagagagatacgcttcttatcttcaagtaaaaagaagagggtttcaactagaagttttagccaaagttttattacgggaaagagatgagtccattggttggcctgtggcatagcccactgggcaatttggcatcatggaggccgccttcttagaatgagtgccaagcttgcttattgtagtttttgcatagagtgaatggtgaagtggaatcgctgtagaccagcgcttctcacaccgatgtgctgaggaatcatctgagggtctaagtgcagggccgatttcggggggcgggaggtgggatgagggctgggtctgcatctccagctacctccctgcaatgtccatggagctagacttctcagcacaaactactgctaagccagcaagccagttcctaaattcctgtttgccaataccaagtagagagttgcccaactgagttaaatgttttggtctgtgttttattttagttcttagtttgattttagcttttcctttgttcttagtatacactttttcaggcactaacgttatcattatgatataaaagctcaatgtcaagtagagtttcttttgcatatttactttctcgtttaacacaaatataacaagcaccccataagtgccatttgcacaacagcaaataatggggacccccatatccctctgtgtagcctaaggtcttttaggggagagacaagttcacaggcaaaaccaggggaaatgcaaggtgtgctggggtctcatgagttacaggagggtctcgctcagccttgtgggggatgctgaatgaggaaacttagcctgaattagagcccccagctggaaacctggcagctggaatcccagcgctctcctctcagccggggttgttcccgcccagaaatatcagggttcctctttcaagcacttaaaagggcttcccattttctaggatgggatggattaacctgataaaggcctcaccaaggtcaaataaatgagcagacactgtatatgctaagccgtcagaaatctaattggcctgtttatgcttcagctagatggatgggatgcaaatgtcttaatgtggctgctgatacatgccccagccgaaagttccagcagtttacattagaccaagacattggctgcttggtttttactaatgtagttcagaagtttctagtgtttacaatagctgtgaggtatttgtatttttatggggattctctctcctttcatctgatgtcaaaacatatatagacatggcagcactggttgcctgttccattttatgaaccagaggaccattaacagatagatatcttgccccccccttcccagaacatcctgagcctctcccctaccccagcccacttccatcagagttgccctagaaatcattagttccagaaacgcatccctgcgtggagtctgtgccctccagccatgcagagtgtctgccagaggctgtccctggctgacaggtttaccttggtattcatgaaggattccagaccttagccctcaatggtgggaaagcagaactacttctgcactctcctcctctgccatggtgctgcggcctttatgtttgaattggctgacgcaaaacaagagtattcagagatgctaaattgttcaacaaaagattggcccgcagagcagccccgccattgtggagtgccctcacccagcacaatgccccagctcccacagaatgggcagcattttctttgcagaaagctggttcctgtgttctaaaaaggttgcaattggagctatttggtaaaactggaactcacagaattctaggcagaaagttgtgattaatgaaaggccagggaaagccttacctgatatgctagagacttaattagagattctgccgtaagatgcagaagcatttcacagtggattagcagcctgcagaagtgggctgcttttcctcctgggctaaattgtttaaattctacacccttcacttcatttgtttgcttctgctgggttttcacgagcggggcccctcggcattgtgtttgtgctttcagtctgcccagccattcaggtcgcattccatatttttgtgtttgtgtaacacattcataaataacattacatgccatcagagttcagcaaattaacaaactaattttttttttcttcccttcatgaactaacactttgggttgtggaatgtagtcctcaaagtcataaggtaaagaaacaaaaagcaaaaactcaccttgtgttgaatgtgaactgcggttggataacagccttttcagtggggtgggttttgttcatttctggcgttgccatgctgctgtgtcgggcacacggagggtggcgcgatccaccctgtcacagcgcgtgttcccgtttcctcttgatctcccaggtatttctttgctgtgctggcgatcctcaccatcctcggcgttctcaatgggctggttttgcttcccgtgcttttgtctttctttggaccatatcctgaggtcagtagtgacacggggatgtcccacgtgtaggccggctgaatgctgtgtttcctgtgccgctcttcacttcgatacttaggtgcctccccacttgctggtggttcttcagtaaacatctcagagtcatgtctgttttcctcttcggtgtactggctttgagggctagagggcgggttcggtttggttcctctaaatcaactgattggcagcctgggtcttacagatctttatacagtaaatgaagactttccccttgagatgcataattggacttcacaagagtaaaaagtacacatcctgcctttccagtgtggagcagaggacagttcttctgctccagctgcgggacctgaaggtcctccaggtggtagagaaggggaggttaatacggcacagtgcgcagggccccagggcagggaacagaggcccctgaaaaataccgtgctttgagctttgagtgtggccagcaggtaaatggacaagaacacttttaacatggaatccccttaaataggtgtctccagccaacggcttgaaccgcctgcccacaccctcccctgagccaccccccagcgtggtccgcttcgccatgccgcccggccacacgcacagcgggtctgattcctccgactcggagtatagttcccagacgacagtgtcaggcctcagcgaggagcttcggcactacgaggcccagcagggcgcgggaggccctgcccaccaagtgatcgtggaagccacagaaaaccccgtcttcgcccactccactgtaagtgactctgcagaaccagggggaggggtcttcccaccgtggtagtgggcctggaggtccagcgggggctcctgtctgctgagcagcacatcacacaggcagatgggatgcagagataaggtacagactaggtcaaatcataggaaataaccagtatttaactctttctcaactatacaaatgccatcttcctgtggtttgatctcataaaggataaaaagggcaggggccagtaatgtcactgcacaaagcagtagcatctatcttgttcagggtgaggacagtttgggcttcaaaggccagagataaagcattcctgccttttttttttttttcctcttaaggtgcatgtcatttgagagtgatattttctaaaactagtagtatttttattgccaacagaggtcaggttttttccttctctcatttctgttaggtttcaatgcccttgatacaacattcattattattgtctaaatactttaccatagcttttggggctccaagctcatgttccaggtggcaggaaagagcagagaggccaagaaccacccctgagttcttagccttcagtgattcaggaggtcaccaggagcctgggagcccctcctcggaggctccaaggacctgggtcctttcctccaccctgtgctccctgggatgggctcagccctgaaaactcaaggaaggttctgggctgatgttgctccactgagggccccctgctgtccctccaggaaatgggtattccagtcccaggccagagcagcccttccgtgaaagagagaaaggccaaccaaataacctgaattgtcctcctcctaccccccgcgacgacgcccgctgagctgcagtgtgaatctggttttgttcagcaggagcctaaaaataggtacaagctgaacgactttgaagttggtcacggtccttttgattgatgtggccactgtgttgctctgtgctttgtatagagacgcagagaccagattcttaacaatcagatggaacaggctgcccgccacagccctccgcagtgacgaaaatgccctctctgctttccaggatgggagccaccggccacacgtggctgtggagcacttgaaatgtggccggggctaccgaggagctggattttggattttagttcattaaaacttaagcaaccacatgtctggagcggctactgtgtttgacagagtaggcagagacagtcctgccagcgtcctgcactcaggccagccagggccgcacacttagcaacttcgggagaggggcagctcagagcctgcagaacctaacagggtcgggggcaggttgtggggggcagcagcttggatgctctgaccacagcccacatttatgggcagcagcagtacgtgtccagccagccgtgtcagagaaagcctggaaacaggattttcaaacaacacgctcctccgccaggtcctggaggttattagaaggacaggcttttcttttgtgggtggaaggcccccattggctccccagggttgactgagtctttggtgaaacccaaggagggaagtgtgggagctgcggggaccatgcccagcctgggataccacccttctaacccaccctcaccctctgcaggtggtccatcccgaatccaggcatcacccaccctcgaacccgagacagcagccccacctggactcagggtccctgcctcccggacggcaaggccagcagccccgcagggacccccccagagaaggcttgtggccacccccctacagaccgcgcagagacgcttttgaaatttctactgaagggcattctggccctagcaatagggcccgctggggccctcgcggggcccgttctcacaaccctcggaacccagcgtccactgccatgggcagctccgtgcccggctactgccagcccatcaccactgtgacggcttctgcctccgtgactgtcgccgtgcacccgccgcctgtccctgggcctgggcggaacccccgagggggactctgcccaggctaccctgagactgaccacggcctgtttgaggacccccacgtgcctttccacgtccggtgtgagaggagggattcgaaggtggaagtcattgagctgcaggacgtggaatgcgaggagaggccccggggaagcagctccaactgaggtgagtgccactgacaagggcagccaagggacctagagccaggcaaagaggaggacaaggttctcctggggttctcggcatccccgaggggctgtggcgctcgggcacatcccacgctggaccccagtgaccttttatccccagagcaaggctgatgtgtccaagctttgtcatccttggggaaccagtccagctccacactgctctcttcccccactgctaacctgcggagggtcggtgcagcgccacaaggacacaaacagcaccaccttttaccaatatttgccagtctttatggaggtgcatgccacagcgaattggtcacatgggaacgcatccgtgtaaccagcccccagatgaagggagaggtcacaagcagcccccagggacacctcaggcccacaccccactctcgtgatttctaataccaaggttactgctgcctgcttttgtactttataaatacggacttgcacacctcttactctgttcaacttccttttttgcttccttttaaagggtgattaaaatctgaagcaaagaggccaaagattggaaaccccccacccccacctctttccagaactgcttgaagagaactggttggagttatggaaaagatgccctgtgccaggacagcagttcattgttactgtaaccgattgtattattttgttaaatatttctataaatatttaagagatgtacacatgtgtaatataggaaggaaggatgtaaagtggtatgatctggggcttctccactcctgccccagagtgtggaggccacagtggggcctctccgtatttgtgcattgggctccgtgccacaaccaagcttcattagtcttaaatttcagcatatgttgctgctgcttaaatattgtataatttacttgtataattctatgcaaatattgcttatgtaataggattattttgtaaaggtttctgtttaaaatattttaaatttgcatatcacaaccctgtggtagtatgaaatgttactgttaactttcaaacacgctatgcgtgataatttttttgtttaatgagcagatatgaagaaagcacgttaatcctggtggcttctctaggtgtcgttgtgtgcggtcctcttgtttggctgtgcgtgtgaacacgtgtgtgagttcaccatgtactgtactgtgattttttttttgtcttgttttgtttctctacactgtctgtaacctgtagtaggctctgacctagtcaggctggaagcgtcaggatatcttttcttcgtgctggtgagggctggccctaaacatccacctaatcctttcaaatcagcccggcaaaagctagactctcctcgtgtctacggcatctcttatgatcattggctgccatccaggaccccaatttgtgcttcagggggataatctccttctctcggatcattgtgatggatgctggaacctcagggtatggagctcacatcagttcatcatggtgggtgttagagaattcggtgacatgcctagtgctgagccttggctgggccatgagagtctgtatactctaaaaagcatgcagcatggtgcccctcttctgaccaacacacacacgacccctcccccaacacccccaaattcaagagtggatgtggccctgtcacaggtagaaaaacctatttagttaattctttcttggcccacagtctcccagaaatgatgttttgagtccctatagtttaaactccctctcttaaatggagcagctggttgaggctttctagatctgtttgcatcttctttaaaactaagtggtgagcatgcattgtggtgtagaggcaggcattatgtaggataagagctccggggggattcttcatgcaccagtgtttagggtacgtgcttcctaagtaaatccaaacattgtctccatcctccccgtcattagtgctctttcaatgtgatgtgggaaagcaggaggatggacacaccccactgaaagatgtaggcaggggcaggtctctcaaccaggcatatttttaaaagttgcttctgtactggttctcttcttttgctctgaggtgtgggctccctcatctcgtaaccagagaccagcacatgtcagggaagcacccagtgtcggctccccatccaaatccacaccagcaccttgttacagacaagaagtcagaggaaagggcggggtccctgcagggctgaagcctaagctactgtgaggcgctcacgagtggcagctcctgttactcccttttaaattacctgggaaatcttaacagaaaggtaatgggcccccagaaatacccacagcatagtgacctcagaccctgatactcaccacaaaacttttaagatgctgattgggagccgcttgtggctgctgggtgtgtgtgtgtgtgtgtgcgtgcgtgcgtgtgtgtgtgtctctgctggggaccctggccacccccctgctgctgtcttggtgcctgtcacccacatggtctgccatcctaacacccagctctgctcagaaaacgtcctgcgtggaggagggatgatgcagaattctgaagtcgacttccctctggctcctggcgtgccctcgctcccttcctgagcccagctcgtgttgcgccggaggctgcgcggcccctgatttctgcatggtgtagaactttctccaatagtcacattggcaaagggagaactggggtgggcggggggtggggctggcagggaattagaatttctctctctcttttaatagttttattttgtctgtcctgtttgttcatttggatgttttaatttttaaaaaaaaaaaaactttgctgatatttataattttgtatcataagaatgttttcctctacagtatttgtcatgccagtttataacaaaaaaaaatgcagggattttatttctattggaaacattacagctatgttttacttttggacagaatttttatttgtatagagtgcttactaatgttaaatagttcagagtatataacatttacattaaggactcatggtaggttttagggtaaggagtttaaaggaaataaatattcaaactgggtctcattgccaattttggtggaaatgagtttgtgtcatttcaattacaaagataaaagtatgccatataatttatttatatgaagatttatttttgtagtgtacatagtagtcatcaagtcttttgacagaagtatatttttaaagaatttatatgtgatgaatccataatgtctggaactttgctgagacatgagtgggcacagttttcattgtaaattacagcaaggaaagaaaatgtttaacagtgttaagagagtcagagcagagtggatattcatgcgattatgaagtgtttattagttaccattggcgacctagcatgcttctcatttcaaaccttggaaggtgaaaatgtacaaactctctaaataattaatgttcaaacactgatagaaattctaacatgaataaaaaataatataacttgttggttatatctttgtttgtagaatgctttttttctttaaaatacttgggagagacagttagtgttggagccatcagaaactgttgtttcatcttgctgaccttgtgacactcttcttttttgctctatctgatggagagagttttaggttttcctcctttttgttttgtataaatagtgggtataagcatagctctgttacatggatgtattacaaagtgggggctgagcttttagtgtgatcatcacccaaatagtgtacattgtacccattaattaatttcttatcacctgaccctatttttgttttctcataccaagaaacttcctaagttaaccatcaaaattagtccttctgtcatctctcttagcatacttatttcccttatgtgatgcctaataatgagggaacataaataaagccaaattcaaagat

SEQ ID NO: 7 - DNA, 53988 bp, Mus musculus
atggcctcggctggtaacgccgccggggccctgggcaggcaggccggcggcgggaggcgcagacggaccgggggaccgcaccgcgccgcgccggaccgggactatctgcaccggcccagctactgcgacgccgccttcgctctggagcagatttccaaggtgcacttctgtcccctcctccctcttcacttttccctcttcttcctccttgggatccctgccacacagacacacacacacactcctgagaaagcagcagacaagtgaaggttgaaaaattcaaaccctccctccggactttggaggaaagggctgtctgaggtccgcagggggtggaggggtgcctgtgtgtgtgcgtgcgtgtgtgtggtgtgtgcgcgcgcacacgccctccctggtgtgccttttccggagctttggaaaccgtcctgagtggaccacctccagggcggccgcaatgcggcactgccttcggccccagcgcctttctcccgcgcccctgcccacggttgcagcctaaagttcggcgttgccacatttcttaacctctttgagggggaggcggagtggagagaggcggagagagacagaggggagggagccaaagtaaaggtggtttccttttttggcagccggttctgctttcgttgagcttgaaatctctgctcccttaaaaattattctcgggagaggacgtcccctctcttccaggtcttgggccgcctcttctcccagcctgccggggaggggggcagttgtaaacaagttgccaccttaaatcttcggtacgggtgtgcggaacggccgggttcattgtgtttacgaggctggctgaaatgtgtggaatccggggaaggcaagcacctagcctggggcccgcaggggccctggccagcaccccgggctccgccgcccggagagcagcctgcgcccagccaggacccccgccggcgccggcctgggcctcttccttgtgcactcggctattttaaagagttcaccaggagggaatgggagagggaggaaagatccttgtggccgccctcttccgagcacaaatagtgtaagttgtagatggctgccgcatttcctaactgagctcccgcggtgtctcctaaggctgtggaaaagagtgtggaccgaactctggctgcggggctcaccacgtggacctcgcgtacagccacaccggggccagatggaccacggttgctgtagattgtccctaagtgcgcttgcgcccttcactcggcctccggggcactggaggaggccacaggctggagcaggacttagggaaggggggacgtttcctgggggttctttagtgttgggcttctcggctaggatcactcaggccctccctccaggtccccggtactaaaccatggctgctggtggtggcttctcagttagcctctctccccagcaccccaagtctcattcaggactggcgctcaggccacgcgtgggggcgccccctggtccctagagtacagttcccacgctgcagtctcactgatttacaaccaaggcccccacgcccccagggcttcctctggatggtcctggttccagtccgagggagaggcattgcgccgcgggcgcccacgccccacactgctgtccagaggggatccccgggcgggtgtgcgggggacagcgattccccggcgttcactcctcccttctgcttcgtccccaggggaaggctactggccggaaagcgccgctgtggctgagagcgaagtttcagagactcttatttaaactgggttgttacattcaaaagaactgcggcaagtttttggttgtgggtctcctcatatttggggccttcgctgtgggattaaaggcagctaatctcgagaccaacgtggaggagctgtgggtggaaggtaagcgatcaggcgccgctagcgcgtccttcgtcttccttccctttactgtgtttgatgaagatatttgccagcatgcacctagagcctccttgtagccaacgagaagcccttgcccgcaccaggggcgactgtttattgtttgcatggctggccagtagccaggcccgcccgacaaaggccgcgctgtaatctactcggggcgctgcgctttgctaggtgtggtgggagagagtggcgggggcgcagaggccctgcgcgggacggaccactccgcagagccgtgggggtggtccccgaagctcgccacagctgcctgcctggctttggcccgggagccgcggcccgggaaaggcctctctgatccgggagcctgagctcggcttctttacccccaccccccacccacccactgtgtctgcggtgaaaggagaggggggtggagggcaaggccacccctctggctgcagggagtgtgcgcgcccccggggcgcgcagggggcgggcgcctcgcgggcctgcggaggcggccgcgtgtgtggtcctgtttttggactgctttcaactctgggggaaaaaaacatttgtctcaaggtaccaaaggggggtggggggagttgaaaaaaatagtaaagcacaaactggaacgcatgtttgcaaagtagattaatgttttctcgcgggtgggggggctttttaaatggaattattcttgggggtggttgcatttgttaagcttgcaagtgtgtgtgtattttaaaagaaagcaagtttgatgggaggggccacgggagcttccctcggtgtgtgtgtgtgtgtgtgtgtgtgtgtgtgtgtgtgtcttttatccaagtgcctcgatttggaaggggaggggttggggacctgctttataaaaggacttgtaaattgaaaataggttttccatttttccccaccccaccaccgtggaatctttttgctctttgtgattttaggctttcttttttccttcgttctttgttagtgtctcagttatctctttgaaaagtaccaaccccttgtttgaaaacattttagcttctggcaaacactgctttgagcatcagagtagttgggcagatgtttcaaccccaaatatttggtacatgcctactgcaagccacccaggacagaattttgaaaccagggttaatgagtaatttcttaggtcaccagtttcaggatgctcttggaaactaaccttccagaatattctgttaccttagctttcactatttttgcaaaactagtggtggagtatttagttggaaagtaacaaaataataatcctaatctttaagagcagagcacattcctgccttgagtagcttggttaatgaatacaagtgtcttttggattgattccattaggtaatttaccccactttgcttccttgtcattggtagttcagagaatagttttattgagcactgactatgtccctggcaacaaatttccttagtatctctagtgttcctgaccctgtgctaggcactgtgatggagacattgcacagtgccctgtgcttcagactacaggaattcaatgcattttcactgagtataggctcccacttgaactgtttcatgaaagttaaattaggagaaaaaaaggccttagtagatgtcttgaggtaggcaggggtactggagagacaggaagcaagagagtattggcgggaacaggccaccccctgcaaggtcctggaaagttaagttaaccagcagaacgtaaggtcctaggcacagaaactgaccagcaagactatagagcagaagttaagctaatggctccacagttggctttttccaggctgatgagcaccatggccttgcaacagtttggtttttagtagttggagatgctgtttccataagttttaacaagcttacagaggtagtagagttttcttaacattgtttcaagaatgatgaaagacttaggtagagcgactccacctgtctaagtggaagatgttcagtaaggaggacacacctgaggcagacacccacccctacccacttcccccctcctgcttttggtatcttgatagtttgtttgttttctgctaatacagctatggaacctagggcaccaccatgttaggcaaatcctccttctctgagctacaccccaactctggctaggcttagaggccttagctcctttctgcattgccattttagtatggctcattggttcttgggcctaacactttaccttacaagtcaataaaatatccgtgactttaattttaggacatgatggtatatgaatgtaatatttcatataataggtgtctctctgttcaaatgagaatcatatgataactaggagtgtggtctgcaggtcccttcatgttcattttgctttccattgatttaggtcatttagtctgacactgccttggcctcattcattcattcattcattcattcaactcttgccttcgcaagaagcatgtttcataaatgtagttaagcgcatcgaggagagcatttgatatggtatcaaatttagtgttcagcagtcagaacaccgcgagtccctgctttgaggaatacatgtctgatggttgtggtggtcattgaattcaaatgagcagcaggccctagcttaggttcctgcttcctctttgcttcagtctcttagataatttcttcgtcactgtcatctgttacaggatgctctcttcagattcagggtgtgattccaggagcacacacagaccaggcaaggggagtcttacatgtccccagctaagcaacagaagaaaggaaagacttccggtttaagggaaaagggaagctgggaaaccgagggacggtttggaaagtttgatattcctcctgtcagtcattaaaaaaaaaaacaaaaaaacaacctgtgatgagttgcctctgtgcttgtgtgtatatgtctgcacacaagcttactgggtgtgttttatcttccaaaatatttttaatatagccaggaaaattaaggaacatcctgacgtccatatcctggccttgcctttccacagtttaacatcctgaagaaatgtagcttatgaatcatccattcaccacagaatcaagaatccatttcccttgctcccctcgtcacaaacacacaccatacatgcatgcctaagtgataacttttttgatctgttaagatgttggtataacgattgcctttcaagaaactgaccattatatgtgaagtaccgccaccctgagtcttaggatgttttgttttgttttgttttgcacgatatagatgtcaacagaagtttacttgtgaagggacaaaaaaagttttgataaagaccctgctgtagttgggcacctactgtcaacaaaaacccatgatggcaccagaaacatagaaacatgagtggttgtttccaagtggaagtatttggtcagtgtgggttctctgtgcccagtctgcactaggcactgcccaaactcctgagaaatgttgatgacggtcggtctgtagacaagtctgtaggtagttagttgccaggatgttggggaaatcagtgaaagcttccaggatggccccaggcttgagaaggtggaggtgagggacattctaggcagaaaaaggagggtgggtgaaggctcaatagataatttgtgggtggccttcagtgttcgctttccagagatagggtgaatgaagtgaggggcaggcatctggtagctcggaatgtctccagggatgggtccatggagagtggctggggcctgcaggcgatttcaagatgctgtcctgggtctgtggttctggtacccttcctgatactggaagtgcttgcatttgcgtctcagggaacggatttatttatttacttttttttaaccttgcccatttttttcagtggtgtttcttttggcagatctgtccttctggtccccctaatcggtttttctttcgtatcccaccccccatccccctccagctttctgtcacaagtttgttgtcaaggtactcctgtgacagaatcttcatttatctccccgagataaaagcttaggcggcctctggcctccttctctgaaaagggcaacaaccttgaaatgactccatattgccttggtgtttggttgttaagggctttaaggcagtatcagggaggaggatgaaaatttattaaagagaaatggaggattctacttaaaaaaaaaacagactcatttgatgggcagtcacaccctcaggtctgcatgtgtgttttctgcatactttgcacagttagaaccaccatccttcatttgctcagccttggcactctagccatttggatctggataatcctccactgtcagaggctttacaccctagagggtgcacatcagagcccgtggactctcctgacaaggtgccagcatcaaggataaccttcccaaccttcccattcattgctagatgatgctccagttgagaaccactgagttgaacgatgaatgactgggtagaagacctgggctttgcaggtctaattgggcctgcagaggtgctactctgtgtgtgtctctgcacagtttgatgaacaagaggaggtcattccaagagcaaatgcacacacaggagaggagtgagcaaattgttggtattccctttgtacttagagaaaaataagtgacaagtggaaactaccaatttggaaaaaaaaaatagcaggtggttcaggtatcccacacacagggcagttggtgggctctgagcctatccctatgctgcccaccattcttgatcactcataagccaatttggaccatggactgtttcagatacatccagcgccgctaggagcttccaggagatgacagcttccagatgtatgggctgcctggtccgtgtgtttaggttatacaccacagctgatgctaagattaatgctggctggagagctagggagattggtctgcctttccacaacgccatgtttatatttcatttggatttctccttcagtgctgacctgcctgtgacccccgggtccccatcctctagggaggaggtggtacttatttgctcttgcttaacttctggctggagggtaaagaggggattccaagttctgaagagaagctcgtgcgtgcagagaggggcttcttgtggctgcctcccacacagtgagtgcctgagtgtaagcccctgagactgtgctttcgctcaggaggtgatgtgccagggcttttaaatacaatgttttacaattattttgaaggaaaccagatttttgtggaagccattttgcatgaaagcctttcacctcttggcaacactgttttgacaatttgggggtgggggtggggaatgaggagattatctgtaaaaatcgctatatccctagtattctcatattgtgctattgtttccagtgtctataggctctgacagactgcttcctcttccctcctatcttttgtcgcccacccttagcccccttgcttctacccactcactcctaaactgctcaagttcatttcctcccaagtgagtcagatctgaaaaggtcttctgtctgggccgttaggaaggaattagtagatgtgttctttttgcgcagtggggtgagagacgttagccccagagccatcatatactacggttatggtgggctctgtggttaaacattctgttaggttttgatcattagaagcgttgcttttcctgatgaggattcctgacattgttgcagacttctaagtaactttttataaagtaacttcaggacctatggacgtgttcttcctgtgtatgtaaaatgggcagagtatatgaagtaagtcttaactcctgtccttcttgtggcataggatgttagggagaattgggccctgtagcatccctgaactgctaagtgagacacagggtgacatcccacccaggctcctcactttcgcctttccctgagtggaacacccaccagaactagtgggaacaggagcttggatagagacaggagaggactgattttcttatccaaattttggctgaccccatagccttagatgtttcatcctcatgaggtctctgttgacatcccagaggtcaaagggccttctgggaaagccagttcagtatggacagaaggaggaagctcttggaatagtctcagtgggaacgttttttttgaagcacagatacatttaggattgccctgtaaataaatacaaacccttcctgttgttgtgttgctgctgctgctgctgtttcctgccagtctctagatttgggcaccttacaggggtttcttgctttcaaatcagatttggatccttttatattagactgatagtccctttgtgttacatagtattaattttttttctgtgtatcagcatctaagtagatgtagcagaaaaagaaaaaaaaagccttcctagacagtaaatgttgctattgagaggctgcctgaatgttggcaggtgtgcaggagcctattgtccagaaagttgcataccatccctttcccctgggaggggaggtgtcgggatttaattggagtttagagcatagttggcaaaacagttgccttgtgttagggaaaccatttcttggttgacagtcaggtacctgctaatgaaagacgactttgttggagggatttaatgtgagaaggcaggaccttacttgtaatcctgaagtccaggtgtgaattgacttgaatagtcccaatgtggatggaagtgggtcccacatgggggaccacatacctgtgggtccccacatagaagtgaggccagtgaaaaagcgggtccctgtgcacatcacaatatgtgctttatattcctcttgttgggataaatgaaaagaagcagctgtaaatgtactttgtttagcagctgagatgctttccagctcttgatgagacatggacagaagaaagcaaaagaaactggaaatgggctttcagtcagtgtgcttaagggcttctggctactagctcagggatcctgcccaaaaatgccagatgtattacatagtcaaggattgcaatgtaaacactgctctgtgccacgaggcagggtggtccatctcctacttaagaaagcaggagacccagaggagcagagcagcccttggctagctgaccactgagcacagtggttctgcttgggttaggggctgcacaatcaagatttggaagtctaaaacccagataaattaacaccttgaaaggcaactaacagccacctgttgatgccgtagagaagcaacttgcctcttgagctggaggttggagatgtggctcagtagttcagtacttgctagcattctcaaagctatgagttcaagtctcaccagtgttctcaaaaaaaaaaaagttgagtatgggggttgggaatgagtaggaagaaagctaagtcctccaagaagcatctttaagtcgtgaacatcatttttgccatctgctcttaaattgaaaggtgagaggaagctaggccaagagaggccagctctcttccggacctcctacagtgttagagaaagcctgctgggttggttcatcttggtttaagctcaagatttatcacaaggctgctgtgatgtagcaatgggaggtggcttaagataccttgtcagatgcatggcttacaggtcggctgtgctttctagagggaagtcaggatactgcaaaaggccaagaggggaggtaaccgggcacctccagctcctgtccactaaggtgtgtttttacagtgcggcagacagttagagattggaaggtctagagagacctcagaacttgtactcagcagtcctcagaagaaccagactcacagttaaggggggttaagaaaaaaaaaaaaaaggaacatgttttgtgagttaggaggaaatcggattttggcgattgttaaaataactgctctaatattttcctgatttaagaaatccatccgtagcatgctcgggaagcgctcctttaagaaatggtaatagtaaaaacaggaaatttgagattacagaaaatagaggcttcttgactccctgtctttccctcctcatcacccccccaatccccccaccctccccggtctgccagtcttccccacccccacctctccctcccacactcctcagttttttttttctcctttagtgggaaacaagaaaccttcacagaatagaggtaaacatcccgcatgcctccttttaaatactctaaggaagaaaatgccaaaatactttttcgttgctgcctacgaggttatttcttctcacctgctgctgcttggtgtgttcttggaacagtattaagttaaaaagatgtcaagtgtgcttcatcatttttaatctgtatgtggtccagggaaagcatgtctgatcccttgttcagttgcaaccctcacttgccaattcgagagctctgaattgggagtatctgttattgctctctctagagatttgggggataggatgaccccttttttttccttctttttttttcttcttcttttttttttaaataggctacagtttcacatgggctccccaaagaaatctctctccctttttgttccccataccaattgatttatcattttgcttgcaagcagatccagaatgttagggcaagaacacactcacttttttctcgtaacttttttgttgcgggccgaaaccagttgacttgaataaatttttctgagccctgaatgtagccagcaacgtggaaaaatcttgccattcagggccgtctgaatgtacatttcagccacacagtaagctgcattggtaagcactggactgctaaatacaaccatttttcgcagcagcgagttagacaggtgattagcataattagcacggagcagctatacatatggtaatgttgagtgtgtaagtgccagctgcggagtaaaatttatgtcaggggtcggcagggctgtgcgaaagtattgtgtcccagctgcaggtcagggccgccaaagcttacctccttttctgtccttggtgaatacgccaaacagaagagaaagacagaaatataacagatagtgctttgatggaacagctcggttctgtgcgagagggacggtcctgtaacattgcacacgcatgcacatgcttgcacacactagcagacatcccatgatattgagttctccacaaatgatatttacctcaagcaggtcttcatgagtggctatggacaggtttaggtggggagatagacctgaggtcgtttcctggaagaattttaggtgctgagctcagctataaaaggagctaaaagttgtggttttgtgcttgtttcttcattccctgctctaccttcttacccacattgttttccttcaccttaaatgtggggagagttccctgctggttggggtactgctttcagctttaggaaaaggggactgccttagacttttgagctttgaccaaaggacaagttttcattggtgggactctgcccaaacttgtctggttagagggaaaggtgtgtgtgtgtgtgtgtgtgtgtgtgtgtgtgtattttaccctcctgtggacagtatagcatttccttttctatctgtaatcactgttagacccactaaatttttcactgataaaatagaaaaaaaaaaaaaaaaactcaaaaaaaccaaacagggtccttgtttgaaaggacagccttgaaccagctcacactttgaaatctgttggggagtgcagtggggtgtttatacattctccactcaaagcatatttttcctctgttctcccctcccacacttttttggttggtggctgaaagcagtggtagtgttgacagaatatagttcacacaccacgctgacccggtgatgaaacttcctcctggcaccagggggtccttatgtcctggcccctaatccagctaatcctgacggcaacaaaatcatgaaagtggcccccagtgctgcatgtgtccctgcacggatgcacagggatgcacagggaaggccagtgtagggattcccagaggccagggagtttgcctgctcatgggaaagtggctagatagaggctttcagaggactggctgacacacatgatgtcagcatcactaagaagggacttggagttggtgtcatgccacagccatggctagccagcaaatactccgggttttgttgctttctgtttttatttcattgagacagagtctcatgtagctgagggtggctttgaactccctactcacaggatcaccaggcttatggttgtgtatgctaggtagtcacaagcccctttttattatttacttaggtttttttttttttttttttttgagacaagtgtcacactgtgtgccttgagtgacctggaacttgctttgtagacccaggctagccttatacttacagagatttgcccgcctctgcctcccaagtgccggtgtttaaaggcatgtgccaccttgccaggcagccctttttatttttttaaaattaagccctcagaccttaatgtaattacttgtgttggtttctggttctactgcctggaggagttggacctccttccttagcgtctgcttttctacagaatgaccgggcttgatttgtagtgcattcaggtgaggtggtggtcttcctagcttggggcacttcagagactggtaagcacgcagtaaggaaaacaggatgtatctggctgggccgtttaggaagtctggaaaacactgtagtttggtttgggtgagaacgcatgttgggtgtccatgaagaaggggccaccaacagccgtaggactgctgtgggccacaggcctggtggcagagtgctggtggcagagcactgggctgacctgattgcatccgacagggccagtgggttggtctcaaggagagccattcagaggaagcagccctggtgaggctggtaacagatggcactgcaagctgccggtttgtggtgtgggggcggggcagggcatggatgcctgcccagtgctctggtgggtgcctgcacattcacgacctgccttcagctctccaaactgtgggtgagtgagtgagtggatatcttttgcttccagtgtggttttgtgtggtcatcctttggggggggggggggcagtatactttcatctgtctgattctagcatttttcataagctcaagatgagaaggaaataaagggttccagccttttaggtggcatcacctcccccataagagagtggatggagcgagtgactagacctcacccagacttttcagccggtttactaccgaagttactactgattcttttgttttcttctcccaatttaggcatgaatcggtgccaaaagctctgtgcagctcttcattttagcagtatgcatcttttggccctctcggtaactagtcacatcactgaaagaaggacctgaagcaactaaagaggggaaggaaaaaaaaatctctgtttgagactgctagataattcacaggattaatcgaatgtgaatgtgccctcgtgtgcacagaccccctctttcctaattggactctgagtgggtactttcaggctgaaagggtcatttgttttgactgactgactttttggggctttgtggggaattaatatcttgtacagatgactgcctgttcccagggctctgtactgaggctgttcattcattgatttgtgcaacagcactcttgagtgtgtgccatgctcctggtttgcctctgtcatttagtcatgaagataagtggataggagatgggtctcccttaaagacgtggaattcatgaggaaaacacaccaccttgttttagaccctcagaccatctttccgtagctcacaggaagctttcggtgaaagctttcattgtgacatctttttcttaggctgtgtgagaggcagtggctgtccagcaggggacgtcaaggggaggcatctcatgtccagagtagactggaagagcacccctccagagggtatggcttgatcgaatgcttcaccccatgcgtccctttggaaaaccatggtggctggtttgaggagacaagggtagacgagcctgactgagaaattcattgcagaagcaatgcttgtactctttcaaagcactggtgtcagctttacatttgtggggaatgagatggagaatttaaaagaaagaaaacttaggaccctctgggggaccagaaggaaaatagagccatggtcccatggggctccattaaagggtgcagcttctttcctgacaataaaacactgaaggtaacattactgccttttaagtgagatggaagctatttatctacattcccaaagcttaaaccatgggtttagtacactctggaatatggaatttgatacttcctgccacgtctgaggtatacacactccctgtgtgagaaggctggcagggtagttggatcagaccagtactgcctcttatatccccattacgaccttcccaaatctccctcctccgagggcagtgctggaaataggtctgctgcttcgatgcttagtgcctgtgtgatacatctgcttcataactgacggaggaacaaccacaatcagagtctccctgatttcactcagtttgactgagctctgcaagttgctgcctcctgcatcagaacaacggccccagctctacaaagtcactaaataccaatttttctttgcccttcaccaccttacgttccctcaggacacttggttttcttatcattgcctgttcatgtcactgtccctatagccctcttacagtggaacagtagaaagggaagaatgcctagtcctgtccaccagtgatttctgctcataacactcaccacatgccatacaccatatggacccagagatagatgctgccttgatgacttgagttgtttaaacaactgtggttaacatgaaatgaacagcagctaactgttctgaagttagccagtcattttggcaacaaacaaacaaacaacaacaaactgctcaattaatacaaagtgaaacgttagaagtatcatcaaggaccccaggtcgagctcagcatacattcttgcaagagttgttccactttatcagtagtggttgtggtcttgcatggctcgtactgattgtcacatcaataaaatttgtaactgagagagggcccatcagttaaaagagcagagtaaacccagtgcccggcactcacataggctagctcacaaccacctgtaactccagctccagggcagtcctctctgagtgcccatactcatatatatgcatagcccacaggcacaccaatacacccgtgactgggaataataaatctttcaaagggagactcacaaatatttgctactagagcaaagagaacaaaaacatgaacccttcaagaaaacatggatgtcaagttgcttttaaaatgcattggttttaacatacaccatagggtggtaatagagcccaagagttacatgttccctactttgagaatcctttctgaatcccctttcacgggaatttagttggagagagtacactgatgacctcagcagtgtgacgtatgccatgttctgggtttctttgtttttctgagcattcatattgggtgctgtagatagtgatagacttctgctctagtctctggggtcaggggtagaacttggtcaaactgtcatgatttgcatggatagatccttgctgtaaatgcagttgtgtgttagggaaggagggactccattgcttgatttttattttaaattgttgttgttgttattgttttatgccccatggtttgaggtcacccaggttttaattttaaaagcaaacatgcacagtgtgctgaacaggatctagtcacagaaagcaagggactggaggggcttgaggtgtagtaacatcaccaggaaaaatgagaagttgagcagatgtggggtggtggtccctcctgtgtggccttgtacttcttcagcagtgacctgaactagacattgactgcttagcttccgggtgcctctacacccacagatgtcaagttcagttggctagttggctggaagtctgtattctgactctggctgtctgggtagtagagaggggtgcaccctctgttccagattgtttcccagagtttccctggtgccgaggctggaacttgggacttggtgattgccaggctggtactgagccacacccccagttctccttttccataacttgacattggtgaagccctcagccattccccttagcattcgtcaagctgtctgcacacttggaatacttttcatgaaccctttgctgcccccctcccacagcctcacctcagagctccgtggtggccttggcggctctgtgagatctacacatagggtcctgccagctcgagattctgcactagttttcctaatgagggcattgtttgctttttagcctgcccccttgtttataaaatatgccaagagaaccaaacaccccactgttctcagtacttacttactttcccaaaccaaatgtttatgtccttgaatactaaacttgaagaaaaaggattcctcctcgtgggaaagtgacaatcactgagttctttcccagctgcagcctcaggcctgcttctcagaaactctgagcttatcagtgactttgggaactagtcagaccgtggaaggcagatcctctgtcccccgtcttcccaggaagtctctcagaacaggcagggctggcattctgagcatggtttgctcccaaaattcagtgtacataagtacgcagaaaggagagccagagtaaaaatagttttaaaacacggtcaactggacctggataccagcatgcctagctttagccaacttgtagtagatccaggttggagtcagtagatagcacacctccttggcctcagtagcaggtccaacgtcacctaggagcttgcagacacaggctgcattccaagtgactcagagcctgacacctgtgcattctggatgctgaccaggtggttctgatggaaccaggttccaggggctgactgcaatagccttggagttcccctggcccttctgacttgaatcacagtttttatctgcccacgggttgtgttctgaacacaatgaccatcttcctgtggttttttttcacagaggagtaacttttctgtgttacctttgctagactacttaagactgtaagttgaagccatcatgaaaggtgctgcagcccacattgtttatgcaaggaagagactaacatggtattttggtctggtctgcagggaaaaggtgtatgtatggagtttccacactcccttcccccttagacacaatctgtcagtcagactgtatctagtagatttgggtgtgtggcagtttacacttaaccatttgcaaagccatcgttgctgctaactcaagtttcgttgctagtttgtcccaagtgagacaacacgagtttgaggtgagttctgttctgatcgatacctatctgtgtttgtcagttggtggacgagtgagtcgagaattaaattatacccgtcagaagataggagaagaggctatgtttaatcctcaactcatgatacagactccaaaagaagaaggcgctaatgttctgaccacagaggctctcctgcaacacctggactcagcactccaggccagtcgtgtgcacgtctacatgtataacaggtaaggccctgtgtcacctgcacaggtacagcaggtaggtcccttgtactggattagttccagcagttttttgttaaccttttagaacccaaggttttacaggtgataattctcagcgctgccactgaacacgtctccatcttgatttgttttatagagttcaacagtgagatagtacacactgaatattccttatccaaggctcttgggacccagaagtgtctcaaacatttaaaagtttattttagagtatttccatgtaggtaataatatattgggggcaggattaaagactgaagacaggattgacatatgtctcagtattttaatatttatttttgtgtgtaaaatggagtttcgtggtatagaatgtcctatttgcggtccccgttggtgtcagaaggtttcagattcttgcgcttttcagatgtcgagctttcagattggagatgtgctcaagcttttttttaaaaaatgtgtatgaatatttgcctgcatgtgtgtctgtgtgccacacgtgggcagtctggaactggagttagagagggtgagcggccatgtgagttgagaaccaaacccaggtcacccgcaagaatgacaagtcctcttaacctctgagccttctcttctgccgctataatgtactttgaatgagtatcttggtgtggtctacaagtgaatatgattctattgcgggacttcttacctgggctatatctcatttgcttaagtttttttccccctaaattgaatatattgttaacttggtaaattatctcttactgaacctttctttggagcagttatgttttgcatagcctcagtcgacaatgggtagggcgaagaggtggatgagagggacgataaagggaagaattcaattggtcacaaagcaaatggaggggtgagcattgagtctctcccggatgtctgagtgaggtagattgataataagagagttttaaaacaaaattaacatctttttttttcttaaatacatctatactgtctattattttaaaataggagccatacatatacctggcccattgttgagtagggcaaggtcagatatttgtagaaaaggggattgggaaatatgggacgtgttcaggaattggtctcctcctatcaggagtgttcagtgtagagtaactgtctggtctagatgggaaagtttgcagtcagaggccctgtggagaacctggtctcccagggttattccacacacatatgctaagacctgcctattctgaagcacttgtctgccatatgcttcaggacaagatttaaggttctgtgaagttaactaagatggactcagagttatgcataggagagaacccttgtctagtgagcacttgcatattttaggtgaaaaaaaaaatgaaaacgagaataaatggagtgtttgtccaaggtcaaagatcaagccgggtttcaaatgctgttttctggattcatgtccaatgaacaatcagtgggcatctttctcaaacaaatgcagagattcagctggtcaggtggtgctgaagacccacttatattggggtgtgtgtatgtgtgtgtgtgtgtgtgtgtgtgtgtgtgcacatgcgaaacacatagaacacttacctgatcgatgggttgataagtgtcggaggggagagataggcggatgaatgaatgaatgataccagggagagaaatacagtcagtacaggtagaaacagacaaagccaggcatgaacagggcccacccttcatacaccaggtaccaggtaagactggagccaggactctacataagggttgttttcaccagcattaggcaaagaaaacttttgattagcaggaaggtagaggtttgaaagggcactaagagaaagatggaaaagtcctagaagaaagtgtagggaagaagatgcaaagtatcaaaaatgcccttgggagaaggcaggtccccctgaggagaccaataaaatataactggtggggggagggggtaatttttattttcattccttcgatttcctgtctttactctttccaaatatccttttatttttgagtaaagcgtctttccctaaacacgttgagaaagccatgagtttgcagaacactctctgagagatactaaaaccaaattaattcgtggtccatgcatttaatgtcctgtctacctggtggaggttgtttataacattaagagcaggaaagtttgtttcaaacgtgaccaaaaataacataagctagtaaaaaagagaaaaaggcaagccagtaatactctcccacctgagtctatcccagcacacgccacactagcccaagctccgcctattttgaatttgtgataattccactggattgttgcaggttgtttaatgtgtgtggaaaagcagattaatggtgtaaacaagattattggtagaacacttaaggaaacaagacttcaagtacttcggtagcattcatttacataaacaccccacatcttaattacgatccagtgttatctattcattaaagcggggcttgccggctttctccttggcaaaagctctgctggttttgacagatgcttcagcgtgatcgaaattacagccttttgctaagtcctgttgttcctgttttaagctcatttccttgcagtaagaagcatttttgttttagtttttttatttctcttcactttccatcccaataggcaatggaagttggaacatttgtgctacaaatcaggggaacttatcacggagacaggttacatggatcaggtacattcttttcttgtttgcagagaggggaaagtgttcaaaatttctggaaacaacccgagcagcgagtgtgagcctaaagcacccctattgttttttgctttgacagataatagaatacctttacccttgcttaatcattacacctttggactgcttctgggaaggggcaaagctacagtccgggacagcatacctcctgtaagtgcaccctcagtgtccagccgctggactcagtgacagtttccggtgtggcttatcattgtttattctgtttcagttttggacaagaaataaaaacattgaaattccatgcgcatttttttgagtgaatggtggggggggaggggatttcagcagaatgtttttttcctcttttcagaaacagttctgtaggcatcattagatttcattcacttttaccaacctatttatatgcaaataaaatgcaaatcattgcacagtctgtacaattttttaaaaactctttataaaatgctcgtagttatgcagaccaacactcagacatttaagaagataatttattactgggttcttgtcacaaggaaatacttcagctaaggttcaagatattcctgcagagaaaaatatggaccctactttggatcatggtaatgatgtttaagcggggaaatgcgctgtctataaaggccgtgataagggatgcttcgtagctcagcagttaggaattgcatacaccacaccatttaaagaatgatggttactcggtgtagactttcttcagctaattatgcgagagttagtgcttgggttattgtttccgtcctggcccgcacatatctgcaaaagcaatttgcagtggccatcccctgaccttcagtgtacagttttagatgggaaataaggggttttgtgttttctggttgggagactctgaataatgacttcagcctggttaagcagctacagtacggtttgaaccacataacaggcagccaaaagcctttctggtctgtcaacagcccagtttccaaagtgagtggggggttaggagtggctgtaatagtggtgatggtagagtcccgggggggggggggcgcttgcccaccccactggcaacctgtgagagggacctcgtaagaatgattgttaaagttagcagatcaaatacttggattggtcacatttaaaaagcagtttttaagcaggcaggtaatattgtaattcattctttcggtttttcactttggagctcagaagaacccatccctccccccaccagctaattttttttaaaggtatcaaatactatatcaggggattaagattgtctgttggttcccttaatttgcaaaccctgcttgccatcctcagccatggattcttttcctgacttaagtttgccagtggctttactgggaaagaacttttatattttctcctttcctttatactcaggccccagagttttgctttaataagcctgtgttgtgcaaatgcagccatgtgccctcctgcctgtctgagccacatttttcatggcctcacctctaatttctttcgcagaggtaagcctcctttacggtggacaaactttgaccccttggaattcctagaagagttaaagaaaataaactaccaagtggacagctgggaggaaatgctgaataaagccgaagttggccatgggtacatggaccggccttgcctcaacccagccgacccagattgccctgccacagcccctaacaaaaattcaaccaaagtgagtttcggggggggtgcttttctgttttcacttacagttctctctatgattttcatcctgaagatttcgtttggcagtgacttttgttcctttttaatttggtgtatggaactaaggttgttcacagagctgagttttgctgtaagatttagcataaccctcattagtcttgctattaatgttttctctgaaacaaacaagcccttaacgcactggatcttaacgaggcatgtgacctgcctactaattcccataattatgtgactgtcttttcattacagcctcttgatgtggcccttgttttgaatggtggatgtcaaggtttatccaggaagtatatgcattggcaggaggagttgattgtgggtggtaccgtcaagaatgccactggaaaacttgtcaggtaatccagcagagtaccccccactctagcctgtgccccttttcttgtacacaggcctttggactcatgtaaatcttagctagctgtccagttcattgaaccttggggaacattagcacaccagaccagcttgcaaagagatcttgtgggcaaggttttctagaatggatttgtagttttcatcagagcagtagctgttgatttcaaggttttggaaggtctgcccttacatagactaatgggataagaaaatccagtattagtgtacaatgagttgaggggtaggagctgatctgtgtcacgcactgttaagagtcaaagaccaggaacagcaagcaaggcaagagttggcctctgtgcagccaggattccagtcagtcaagtgtcaggtgtttcgtctgaggtttctccctcgggacactgtctctgggatttctgcacacagagagtgtgtggctgaacccagtgtatctggtcaagctatctactcccctctccttcctgtttcatggaataggaactgagaaaggaaattgcttattaatttaattttggggtctatctgtaaaaggtttgtaaggttacacctttaatatggtacatcaatctattaaatacaataaccatcctggtcttgtttgaatggaaacatgtgcacacagaggagggaagaagtctgagggaaaactggggatgcttgcttgggacttcacattgcacacactgcttggccgctgtgctgaccttccttctgtccattcaccttgacagcgctcacgccctgcaaaccatgttccagttaatgactcccaagcaaatgtatgaacacttcaggggctacgactatgtctctcacatcaactggaatgaagacagggcagccgccatcctggaggcctggcagaggacttacgtggaggtgaggccctgcctagaaatcaggacaacttgtaactgagcccagtcttcctttcctattgaaacattgtttcccgttaggtgtagtgggatttttgtttgttgtttagtctaatagtgcagcccaagcagtctcaaaattgctgtcctcctgcctcagcctcctgattgctgggattacaccactgaactcagcctccatctcttcaatacaaactttaattctaagcactgcatgaaatgcaagcttgattttctttatccagccattgaaagttttgggagtagtgtctgaagagacactcattcttagttggggttccctgaaacatgcctagtaactattgtagatctcagagcccaggcataacagtgcaaaagggggagggggaaaacccacactgggtgaacaagaaaaggtgtctggaaaacattagcaaaacagaatctattcacagctgcaggccccttggtcaggtcatgtgacaggaggctggcctttgtttccccaagcagtgacctctatggagctgcctgtgaccttttgaaagtgtccagcaaatgaaggggtgggggggaggtggaatttataagaatgagttgttttggattaaggcaacaatcatctggctgtggagtaaggaaccttagagttacaaagtaaaacactctgggtgaccacagaccgcctgccctcccttgagcacagcagccctcactgtcaaaagcaggtttctcttccctcaaccgctgtccacttgattgggacctcgtgttgctgtatcagcagacggttggaggcacatggtggtgttccccaagtctgtgctcttgttgctttggctcctgggaatttagctttgtcttgtggccacaggtggttcatcaaagtgtcgccccaaactccactcaaaaggtgcttcccttcacaaccacgaccctggacgacatcctaaaatccttctctgatgtcagtgtcatccgagtggccagcggctacctactgatggtaattctgggctcccgtgggttggtttgtaattttactttgacggtacctgttttttaactgcccctaacataaaagtatgcccaagacggctcagccttggaaggattttagaagtggtctccaccctgttgtgtggtgttcatagtgtaaagttttgtctttggagtcgataggttctgcctttggatgtggggcgatggggtgggtaaaacgttagaattgttacacattctaatgtttggctcttgttttgtgtcctccccttccctctctccgattcccttttgtccccttcccccaatcgttctgcttgcagcttgcctatgcctgtttaaccatgctgcgctgggactgctccaagtcccagggtgccgtggggctggctggcgtcctgttggttgcgctgtcagtggctgcaggattgggcctctgctccttgattggcatttcttttaatgctgcgacaactcaggtacttcaggggccattcgttgctgtcctctggccaacacacctttcctggcctgccggccccttcatatctatcttacctaaagcgtttgtaaaaccttttctgtaagggaaaggggagggctggctctcagtagacccatctgatatatgggctaaaaggctggagagctggtgaaatgggtcagtggggaaaggcacttaaccctgcaaacctggtgatccgagttcagtctttagagcctacataaagatagaaagaaaggactctacaaaattgacctttgacctttgattagtcatgtggcggcatatacacatacattaacaaagcttttttttttttttttttttaattccagggatgttcttggtagatagagacatttaattagaatagtttatatcatttactcaaactctgactctcatactccagtaggcattagaatcctttaaaacaaagatgactaggcccagtctgccagaaggcacaatcttcaattctcagcatttctcactgctggtctaggggtcactaggaactagagctttaggtatgagttagctagcttttcgcttttaattctaaaacaagtgcttgtacctggacctcagaaatagaagcttttgtggatgcagaccatcagataagtaggatatccaccaaattggcataagctcccccttggcacctgcagggcactgacctcagtgtttccctgtgagatcttacctggccaatgctgtcgttgcctgtgtttctgtaggttttgccgtttcttgctcttggtgttggtgtggatgatgtcttcctcctggcccatgcattcagtgaaacaggacagaataagaggattccatttgaggtaaaagcaggggaaagcacaatgtttgggagacatccaaagcccactcatttccttagtatttgtcctgtagttttcccaccatctggtccatcccaagtccaatgtcactgtgagagatgccgagagccccatttagggtgcccatggacatgtgggtgccttggcagaatgcttcatgttcccaggccatcccttcccaaagcccacctttgcttctgtcgttttctctggctatgcatttgcatttaaataatcattgtcttgagctttctgattggggcaatggtgaccatgagggaggaacccacgggacccagtgctggggtcaaggtggttgggacctttccctgcctcaggggtggtacatccaagtattagaaaccaccgctgtgtgctttaaggggtggctaggacagactacagccctcagcactgtggcttgtcatccccaggacaggactggggagtgcctcaagcgcaccggagccagcgtggccctcacctccatcagcaatgtcaccgccttcttcatggccgcattgatccctatccctgccctgcgagcgttctccctccaggtgagtttgccatgggcagatccctgtctgtggcttggctgttcagcatccttgctctgctctattagagttcaacacttaggttacaaatcaggtgcatggcagcagcatttgggtctcggtgctgctgggtgggtgctccaaggcatgaccttggcactccttccttccaaaacaggctccaaggttctttgccttcttagaccttattatccagaagctgttaacctgtggagtgcactgtgcagtgcaggtcagtggggccaaggatactctttagcgtcctcgttggctggcttctacttctagactactccccttccagtcacctcatatctcctttccctaaacagctctggccccacctcaagaccttagaagaccctactggtatatggcaatagggttgcctgaaccctgggcctggcagttactgtcactcacactgtgtaacggtgtgttagccacttggctatgtactacaggggcacctcttggggatcatgtttgccactgtcttttctttgttggccttttgcattctctcctgtacctttggttccttactccttaggctgcttatgcaagcttagagtgcctgccactcaccatgtgcaagggctgtgttgctggtctcattactctacccacttgctccagccgtggggctctcatacccaggcaatcagcctgaagagtctgtttccaggtgtgagaggggcagctgtgagccacatacctagtagttgtggtagtccgtagcaagccagaagtggtcactatagcagaggaagcaggctctcatgtagttgatgacacagcagaaggtaattgaattaaggtcaagtttttaaagcgacaactgaagagaacgtttgtctccattttccttagggtaggatctacaactcactaggtggccagctgcttgaagaaatcacttggatgtgtttgtgtgaaactgttgttaagtttgtactcctccatgctacctgctcaggtgctagtaaggactatgatcaggaagaagaggtgacaaatgatcttgtgttcctatttatattcttggatacacatttgtcacttacgtagaaacaatcccataggtgtttgaacttagtctccctgcctgctgtgagcgaggcgcaagccctccttaacatttagggtaagctctgagactatgagaagagtctctgtcagtcacaagtaccaactcctgaggaggaaaagagtatcagaaagaggcttattataaatgtttatgaaaacagagataagagtcggttagtgaagtcagatctgcttctggacagagacccaaggagatgaaaatagccaagtgctctataataggaaaaataaaggaaagatacccctggtcttggtggtaccgctggcctttcgggaggctgttttgcacaggcatgtgtctgttgcatgctcttgttcgtgtgcagctgactctggaatggcaatggttctggagtcctttaatagtgggaccctctctgttcacgctgtcctttccactgcctgtccccttgctgtgcgctccctgctctgctctgtcagcttgcacacacgtcttctttccctcattctctctctcctctccactcagtactgatggttcatcaaggcaaactctcctcccttattggtatcccacggtgtggtctctgaaagctacgttgagttttccctgaggatagccttttagaaaatccagtacgctaagttctcccaaggtaagcaccttacagagacaaaaaaaaaaaaaaaatagttctttgcattttggggtctgtcagagcacccagagagattctgtgccgcaatgaaacgtgtaatgtttggattttttcttgtgaaactttaaagatttatttttttaaccctgacttaaatcatagggttcccactctcaaatatctttcactgatcagtcttcactgtggtgttagttcctcagtgaaaaacccaaaccaagatccttactcattctaatgatcaagggctgctttttagttgtcactgtctctctcttactgtctttctctctgtctctctgtgtgtatgtgtgtgggagagagagagagggagggagagagagagagagaatatgaatgaatgagaatgtgtgagtgttatatgctcatgtgcatatgtgccagagcaggacatcaggtgtgagcccccattactttctgacttgaaaaaagggtctttcactgaaccagaggcttttcagccacctgggtcagctggccagtaaactcctgctagtttcctgtctccatcccagtgctgggattatagacatgggaagtagcacactgttcagctttgtatgtggatgctaagaattggttctattcaaaacttttcctgggaagatgcagcacgcacatctactcatggcccacagggaccctatgacagagccaagtatagataccaccaaaatccaacatggtgaaccattgatttttattggttttactgatgggagtatgggagagggggctatttacaggagcagcaagatttgaagttatagttagaaccgcattaccaaagcttacaccagtgtgcatgaaagctcgcaaaggtgggaacccagaggacattgaacagcctgcccacagctcaacaagttggacggggttcctgcttcttccaggcagctgatctggtctcaaacttttttgtctgaaagtcttctttggagcctgactctgctgaccgttagtttcagggacttgctgaagccatcttgagttgtttaccttcctgtttaaagagcttcctggaggatacaatgtttcagtcttggaagaagctgttacacaacaaaaattggactcaggttttcagagtgactgctcttacctactgggtcacctccctattcttccactcctttttgtttgtggtagggacattaaaacccaaaaccccacaaagaagtagtctaccaccaaactacacccttacccagtaaaagggtttacgctagggccctgccataggtgcctcagctgggccactgtggataatggagacagcttttctaagtagatttcccacatcctgattactgtgggaacgcagtgggtgcacactgacatctggtctcccgtatctgtcccagaactgagtagcttgagtcaggattgtctctcctgtcctgtgtcttccagccatgtatcctgtctcttccttgttcctcgtggaccttttttcatcttctaaacccttctcaggaagcctttttggaccagacacactgtacaccccaacccacgaacactctctctaagccactgggaacatctcagcctggggtgtgagttgtctgaggctggaaaggcatgttgtccgtgcttctccagcagccagcacagtgggatgtgtgtcctagtgctcccgtctcagtgttggcaggaacttctcccacttacatgctacttcataaggaagtcttcagagcccggcatacacatttgagccatagttaacctgttctgtggaaattgatgaggtccttttacaatcacctcaggaaagggtatgtagaagacttggctgttttatggctttgtagacttaacaggggactctttattgaagggtttctctcctggctgctgtttcacctggaacataaacccacactgccttcaaaaagccctttgtgaagtgtctcttggtttgtaactttgggtaccagtgatggctaggcttcctctcctcaccccaatccatcttcctattttgcaaagttttctgaagctgtccttttgaaagctccactaggaggttttgctttttaaatgaatttctgattatcttggctatttaacattctccttcaatttttcttctcagaacaggactgcccttggatgtcctgtcggctttgcctaataacagcccaattttgcaatttttactaggtcagcaggagatttgcccaagtgctgcagaacctagccccgtgtttattctaggttatggagtggcttttgaattcgtacttatttattctacaggctataatcacacactggaatttctttctgcttaactctccttgaccttggcccttactcactaacacaccccacaagctgctcctggggctatttggaatgtgcctattaaaaacctcaagggacacatgtgaaattttatttcaattttcttaaaggtgacactttgatagactcgatctactttacatacagattattcatcagggtcatgggatctcttagaaacgttctagaaggaaaacgtttctttttggtttgtgtgtgtgtgtgtgtgtgtgtgtgtgtgtgtgtgtgtgtttgtgtaaaatattctaagtcctttctgtgcctggctttgtagagaggtagcttggtcaggttggtaagtccttactggtatcactccaatctgtaacttctgaggttcttccattttagttacacatggactcttagtggtggaagagcctcctatctgctatctctgtttcagaatatctttatatggggttctggagagatggcccagtggttaggagcatggcctgctcttccagaggacccaggttctgttcccagtcccctcataccagctcacagctgctttcagtcccagggatccaacaccctcttctggcctcggaggacactgtgtgcacgtgatgcacatcctttcaggcaaaccctcgtgcacatacataattttttaaaagtcatctttgcttacaaagctacaagctagctaggaagcgtgttgacggtggccattgtttctctgctccaggctgctgtggtggtggtattcaattttgctatggttctgctcatttttcctgcaattctcagcatggatttatacagacgtgaggacagaagattggatattttctgctgtttcacaaggtacatttcctggtgcctggggcctgtattctttgggtgtgtagaaatactgtttagggaacagtctgtgggcttcttcaaaagggaatgtagctaatagagtttgggttagtgtgatgggactaaacaaaggttttagatacaggcagggctctggggcacacgtaagctcccacactttccccatcccgcacctgaatatgcccagaatcctcccctagagatttctgctgcatgtgctcatttttctccatggtaaacctgtgtttgtttatcaatgaacagtttggtgggtgtttggagcagagaaatgtcaaaaactacctgttgagaacacagtggggggggggtggatttttgtttttagtttttttttcattctttggcattacattttgaaggggtttattatcgttatttcaggatcagctagctatgccttggatagtttgatcacatctctgtccagagcagttaggtgatccttgcaaagctaaggcagtgatatcaatggggagcatcaagtctccagcagaaatgcaagccctctctctctccgtcatctgtactctgggtgatgtatgtactacacactatggtgctgtttatcttttgtctttcagcccctgtgtcagcagggtgattcaagttgagccacaggcctacacagagcctcacagtaacacccggtacagccccccacccccatacaccagccacagcttcgcccacgaaacccatatcactatgcagtccaccgttcagctccgcacagagtatgaccctcacacgcacgtgtactacaccaccgccgagccacgctctgagatctctgtacagcctgttaccgtcacccaggacaacctcagctgtcagagtcccgagagcaccagctctaccagggacctgctctcccagttctcagactccagcctccactgcctcgagcccccctgcaccaagtggacactctcttcgtttgcagagaagcactatgctcctttcctcctgaaacccaaagccaaggtaactctccactgtgtgtggcttcctgccgtgaaatatatgtgaatgattggcatcttttggatccatcaccatgtgcatgtgtgctacagggcaggtccaccagagtcagactgcaaagctcaggaccactttaatgagcaggttcctacagggaaaggttccatggagcagatattaatatgaatatagagaagctccttactttgtaagcatttttcttcttcatgcgattatctaaaaagaagatttagtgtggctttatgtttttcctgctgaaagactgactccctaatatgccgtgctcagcctgggctgatatgtagaaggaagtaaagacatcggtcggtaccggtctaggagaaatcaggaatgttcttctgggtctcatccagtgctctcctttgtctgttacaggttgtggtaatccttcttttcctgggcttgctgggggtcagcctttatgggaccacccgagtgagagacgggctggacctcacggacattgttccccgggaaaccagagaatatgacttcatagctgcccagttcaagtacttctctttctacaacatgtatatagtcacccagaaagcagactacccgaatatccagcacctactttacgaccttcataagagtttcagcaatgtgaagtatgtcatgctggaggagaacaagcaacttccccaaatgtggctgcactactttagagactggcttcaaggtagaactgtggcaggcggccattgccttgcacatttctgtcattcttgggtcttactcatgattccctttctgagcaccctgcgaaggtgttctgtcataatggaacagcatgagtttcttattgctctctctctctctctctctctctctctctctctgtctgtctgtctgtctgtctctgtctctgtctctgtctctctttgtttctctttgagacaggatttctctgtgtagccctggctgtcctggaactcaactctgtagaccaggttggccttggactctcagagatcctcctgcctctgcctctgcctctgcctctgcctctgcctctgcctctgcctctgcctctgcctctgcctctgcctctgcctctgcctctgcctctgcctctgcctcttgagtgctgggattaaaggtgagcactaccacatcgagccagttttttaagatgtagagtgtgtgtgtgtgttttgcctgcatatatgtattgtatctgtatcacttatgtgcctgatgcctgagttcagaaaaaatattggattccttggaactggagttgtgggtactgagaaccaaacccagattctctgcaagagcaggaagcactgagagcctctccagtcttgttttatatgaagcccacttctctggcaacctcccagctctggctttttgtaactatctgagcaagagcctgttaactcatttaggaacatacagtagggaaaggcagttcgaaccagggagcctggatgcttctgttgctcttttcttctggatggcagctggtttggtggccaagtgaaaattagagtatttggtgggatctctgagctgtctgctaaattacagcctactgtagccgtggattttgggtcagctgttgtgtattttgactgtcttttcaaagttcacccatatactttgggcccagagcaaatttgagtggaaggtatagagaaggtttgctctccccaagccacctccccgtgtgtcaacatccaacactaaagtggtctgtgtgctttacccttccaactgaccatagacatcattgacttccaaagagccttatatggacaggccatccttccatctggcgatgaacttaaaaatactttgctacatcctcgtatttcacaaaaaggcaaatcctaccccctcccccttctccattttatctgtccatttgtgtgtgagggtgtgtgtgcaccatagtatccatattgagatggacccaaagacaactaactgtcctctttccaccatgttggtcctgcagatcaaactcctccggtttggtgacaagcaccatcctagacacatctctaggaaatattagttggcaggggaacgctataagatggtccgaatttgcttgtaactacacgctgactgaatacgaatggcgttcagtggttcccaaaacatctgttcatgttgacatgcgcaaataacagtagacaatcaggacgattatccacacaattcaagcaaaattactcttactggagtacccaaataattctgactccagtatttgttggcttagattatcccagtggctgtcttaatttcccacttaattaggcttgtttaaacacatatttaagtgcccctctttttgtcttaagacagccactttccctttgagtttgcattaggcgataaacaacttgatttgacaggaattggtgttgagcagtgactagaatttgcccatacctgtgcgccatgtccgactttgggagatggtggagaaagcataactttaccctggagagtaacttgaattcccttgactttttagtttcttactctgtttcacaaggcattgttttttaagtagtgaattaacaaagagccaaactgttgagagagtctctgcttttccacagtcccctccccatcccaactttcaccaaagacatgcaaataacttgccaccaatgacaaagaagtctgctgtgctgagtggaggagggtgttgtagaatgctctctccagtgtcttccctttggtgaggtaggccaaggttgagaagggcaagcacacggaagacccaaatcattcagatgtatagatatcggtatatttgaaccacattgtggctgctaagcaaacaacacccagccccagacatttgtgcccatgcgtgctaagatggcaggataccatgcagccctggggctgtaatgccttctggatcctacagagcactggtcacagacaccagggctctagcttgcagaggcattatttagacctcctgagtggttggttacttgggtctttgaacatttcttccaaattcttgtttatgcttttgaagtccgttaaactttgccagctcactggtgtctgtcaacactccaatagttactttgtttttttctcagtcctaaaacatacttaatggctaatacaggccattttgaaagtcagccctgggttagaacccaggaaagcactctccctttgagaaataatatctcgatctctcaggacatctgtggtaaagttggagatgaaatctaattgcttcatgtacagggggtgttcattttttattcacactgacttacatgaacaaaatacattatcatttcatttctaggagcttatatacttgggtaatttcctttagcctagactcaaaataattttccgtgtaaagtgtttctagttaaagaactgcagctgaaatgtaagggtttgcagtaaactgttggaatgtaaatgtcagccaaggggcaagtgtttggtgtacagcctgtggaagttcagattttcagtggtcagtcagaaaatactaaaggggccctgaaagaactttactgttggtgaccatagctggagagcacattagggctagcattgtaggaaattcattctaagttggtggcagggcacatgcacagatccctaggtattgcagaagtctctgtgactatacctttgcaaatatactctcagttacctgctttgacagcagatgctgctaagcttctggtattttccatgtattttgcaattacttctaacagcacactgtacctgtgaaaacccactctaagtaatacatgtggagtatggctggccttaggctgcctgggctcaaagcttagctgtgacttgtcaccaggctcagtcctctcatctgtaaaatgggaatcatgattggttctctcaggaggtaagcagctgaaggctgactacctggagccaagaggcccacataagtctgaagtaataaaagctctcctctctccttgcttggaaaacagaccaggattcttgtagggggttgggggaccagttgtctttctctgtttattttatttttccagagtgggagtggggaagagctttgggtcttaaaataacatctgtgtagagtagtgctgcctctctgtgcaaacctgtgaaaagctgtgggagagattgccaccccggtggtccctctacccagggcctgtaggtgacccagcctacttcctaaaaacaaagcccactgcataacaatgctggagggaggcttttgttaaaaaggctgctccaactctcccctctctctattcccaaacacctcactgtgctaagcattcacagaccgtgtgtttttgttgttgttgttgttatttagtttgttttagtttctgaagtgcccttaggcaagtcctgaaatggaataaaatgtatctgagtagatgtgtctgctgcagggggtatcttgagcagctgggacttggggggtctacttgggaggtgtctggtgtgcagtgaggctggtgggggtattggggagagcaccccccaggcagtgctcagctccaagtctgggtcaccagggccctgtcttctcttccagcatgcttacctctttacagctgtagccaaactgtcaggtctataatgatggttttgattacagcggccaagcccagctgtgtgtggtgagctggtgggctcgcccatcccccaagcccagaaggctcactctttcccgcccagggcttgcggtttgtcatctgttctcacagagccccacagagcctccctgtgtcggcctgtggcttcctgttctcaaactgatccaaatttcccctgtgaggtatttccttgtgctccctctcctccacacagccctccactcagaaggaagggtggcagggaggaagagaacagggggagagtgaataggtgctggctgtgatctttacatggcctggcagcaggtcccacagcttgtattagctgcacaagtcaaacagatggatcagattgacagagctgtgttccctcaccctggctccaggggcagccaagttgggagcttgaatgtgattttttttttctgcttggggtggattttctggtcttggtgaggaactcctgacctatctccctgtgtggcgtgaaatggttaatggatatggtctgtgtcttttccccttgctttctcttgctacaattagtcaggaagaagggtggggggaggccaacctggcacttgggaggcaggtggatttgttagttccaagctagcctgatctacatagtgaagtcaaggtgcaagccaagcagagatacataatgagacctgctctctccctccctttcccactctctcattcagtcttccttccttccttccttccttccttccttccttccttcctttcttttggagaagacagggtttctctatacagccatggctgtcctggaactcacagatctgcctgcctctgcttcctccgttttgcaattaaagtcatgaactgccgtgccctcctctccttttcttctttttgcttgttttctttaaaagatgtgtgtgtgtgtgtgtgtgtgtgtgtgtgtgtgtgtgtgtgatgtgcgtgtagaggccaaaagagacagttatctctagagttggagttgcaggcagttgtgtgccctccagagtggattctggggtctgaacttggatccactggaagaacagcagctctaagccatctgtcttgttcctgggctttcgttccttagtttaagtaaagtgtgtgtgttttattcttccctctgagcatcttttgattagcaccacagttcattcactagcagatcaggagtccttccttaagtgagagaaaactgcagacaagtcgcctaccctgcctcctgtcctcagcctaatccctctggaccaaagctctcaggccaggttttaatgtgttgcacatgtttctgtttctaggacttcaggatgcatttgacagtgactgggaaactgggaggatcatgccaaacaattataaaaatggatcagatgacggggtcctcgcttacaaactcctggtgcagactggcagccgagacaagcccatcgacattagtcaggtattcccgatgtttcaggacactgagcagttattacatctaggcagctgacccatggaatagggctggcagtgtataatcccaccttgtgtcccatgagaacctgaagtgataggagatggtaggaagaagcttgaactggctctgagctagccagaaaacttccctttccctgcctgtcaagcaagaggaggggacagacaggtgttgcctctaacacaatagcaatgggcaaaaatacagtatgttaacactgtgtgcttttcccagtggtgggaagcaccaacccatactgggctttcagagtttattgaacactgaaatatgtgttatatgaaaatacattagtatagtggtctcagttatattcagacatgggatggtgggtttattggagagcaatcatttgaacagaggttgggtaggtgggtggcttagaagatgagcaaagtcactagaggttgaggatggattagttatttaatgctttctggggtcagcttggcctcagcagtagatgttgagtgtaggtgtgggcagctacctgctcttgaatatagcaaacgtaagcaggcgtagaagcatgcaacctccccatgcagctcacgccacagtatgttgaaagctgttcttttggatagtatcttgtattcaaagtacttggattctagcatatcccatttggtttcctcaggggttgcatttgcaccctggatttggggtgagtcccaggcctctttgctcttggtagtttcctatcctcaggcagaaaacatctcgttaatgatctccttgtcagctctgcatattgtcctctctcccttttgctggcactaaaggtgacagatttacaaagtacattgttcagagtcattgtggagtgtgaggtttgatgctttatagtggcgtgcaatgagctgtcgggcctgacaagggtacggcagaccagcctgcctgagaggatagcagggacggactgaattggagttagagatcagggaatctgtgggacgcctgtggtggggtccaggcactgagatatcatttcctctcctgggggaaagaagagcacgctgcatacgtagaggccaggagctgtgctatgtaattccagagacagtctgaaaacctacagttgttagagatcctgtggattaattgaatccagatccctgcagtctactctgcctgtctctcccccacccccccgccccgcccggtctcttccacgctctttattcccccactcttttctctgtgaactcctgtcactggtgttcgttaagaaagaccaaacgttggaaggaggcctgggtctaaagtaaacaggaaatttttttgatgtatctaccatatatataatccacactgtgtctcgtagtggctttgacccaggcaaagcccacaagacctactgtagagggagcagatgtggccactgaatctgaccaaggtgcagaagtaatattcctcagaagggccctggctttgcatctttgttttcctgttctggcaatgggcttttttttttttgaatacacaggtcatcagaggctgtgttagcaaaatttatacagatgctgttttattttatagctccctccatttactgggtggtatttccttcagtgttaacaacagaggttaactgcacacagacgtggatagggatgcttgccaggttccccccgtgcccctcctataaatatgtttttccagattgttcattccccgccttggctgcagacagtctcttttagcattcagatttaattgcttcagccaattcctgggccatctgcggaaccgtgtgtttgctgtaaacacactggtgattgcatcccccaataagtgtattttgaaagacatcaaaggtattcttgtgggactggggcactgtggaggcatgggtattctactgcttgttggaaatgtgggtacaaagccttgctctcccagcaggagtgtgtttaccccacagcccgccctgaatgacctctgttctttccgttttgtagttgactaaacagcgtctggtagacgcagatggcatcattaatccgagcgctttctacatctacctgaccgcttgggtcagcaacgaccctgtagcttacgctgcctcccaggccaacatccggcctcaccggccggagtgggtccatgacaaagccgactacatgccagagaccaggctgagaagtaagtagcactcccatcctgagaggctgggagctcttggaggctccttcagtcactgcctggagctcagcaacagccccatccgttgacactacccagggttcaaaagtgtaaatagaacaatatatttcttttgctttaaaactctgggagacagatgagggtgtgtaatggaacacagccctgcgctctcacgttgagaagtgagcgtttctaacccccttttgcagctctaactccaggcatttctcctgatgccagctgttctgtgtgaaagctgaagatacctccacatcgagtgttattttactatggccgagttttatcgctagggattgtgtgtttatcttaactttgtcaactgtggtgttgaagggatttttggcatgttgggtcatttctataagccaccaagcacctgtgctctgtcagagctgtttaaactggagaggaaaagtctgtatacatccgtgctctcctcttccctcccttcacctctaagcctggggtttgaaaggtggctctgtgtggatttaatttgcatttatgttagagatgttagtagcagcatatgctttggatattgggccaagagaagacatgagaaagaagacgggtcctttttgcaaccagggcaggcagcggtctgaactcccctgtacctaaaccccagagcctggtggcaagcagccaaggcttgtcttaatgaaaatttatagttgagattataatagggcaaatcatgtttagcaagtcaactgaatgaccccaaagggtgtggaacacacaagggcagtaactaaatgcttgttgtgtgaggatttcttcccctgttgctgaagacttagcattgcattagagaaacagagaaggtttaagttgctaggtcactaaaggacaagaaagtgtagtatttttttttttctgcaaggcacgactctgggattattttggtcaaggagaacagattgatgtagggacaaaaaaagcatttccaacttttaagtctcatggcatttagacctcacaaatggtagttgctttggggataataaaaccaaagccttaagggaaaaaaaaaaagcatatcatagcagatcatagaggccttcctctgcccacccaactcgtaccccctccccaccgaccccacccccaccccaccccacctcacccccgccactaccaaataaaaatcaaaggctcaagaggtggctcaatgcgctaaccaaagaagcatgaggactggctttgaatttccagagcccacatgaagctgggtgtggtagcgtagtggtaaccccagtgcacaaggcaaaaggagaaagggggggtaggggtgggaaatgttcctgggattccagagccagctagcctcaaatatgcagcagcaaacagcaagacactttttacagggagttaggaaaagattgacatctcagattgtcctctaacctccacgtgtgtgcctgaacacatgcgtgcgcgcgcacacacatacacacactgagaaattttaaactgacagggaatattgtaaaggcccagaaccatttattgtttgttcttttcatggacttttgagaagttctcttctctttacctcacacccagtgtctgtcttcaccggggtccccagcttacctcacttctcctctctccagtcccagcagcagagcccatcgagtacgctcagttccctttctacctcaacggcctacgagacacctcagactttgtggaagccatagaaaaagtgagagtcatctgtaacaactatacgagcctgggactgtccagctaccccaatggctaccccttcctgttctgggagcaatacatcagcctgcgccactggctgctgctatccatcagcgtggtgctggcctgcacgtttctagtgtgcgcagtcttcctcctgaacccctggacggccgggatcattgtgagtttattacaagggtctttatggaagtcagattcctttcaacacagctcttgctgcagccgggaagttttgtttatgtctgggcttttggaggaggagggtatggattgcatcatctgtaagatatttattctacttgaggtggtgaggttcatggggatccttctaacgccagagtctgtgtccctctcacacagcattaaaagaagtgcacaggggccccgggggtggggggcagggagggggggatcggaggctttgtaaacaagctgcataaaagtgctgaaaatttaataagttaaaaataaccaaagtataaataattttctaattctaaagtctcattgtgtgccctcatctcagaaagttcttgtgtgtgtgatgtggaagcatgcatagagaagtttgtacagaagttagggcagggcggggaggatgacactttgagttttgtgcggcgggtaccatggaactcctagggaaaatggtgaactgccactcacagccagtttggggcaacttttctccaaatcaccctcagatttgaagtgagtctgtgtagagtctgaaatcctggcattgcttaagctccatgagccattgtatagcctttaaaaatgccagaattataaaaacaggttttgtttctgtgccgagtctgggcagcaggtacattaaaggtaaatgtttagtctgcaaaagtcttcacagcactcaggccgtggatggtgtccgtctttaactgtttctcccgacctctgccaccacctactgtcctagccccaggcaggggatcattttcttttattcctccatgtggtgagagtgaaggagattcacagtgaacgcctcttaggttatgaaacagttttctcctccttggcataaaaaaactccttgtaaaaaaatctaccttgtagattttatttttttttttttaccattcctccacacatctttttgaaaatgaaaaggttcttcgaagcctttgcttaaacctgggggtctgctttgtagattcttgttgctccctatctcttcaaagcacagaggccctgggacctgacacgtgacctgcagtggcctaccccactctccagtctcttcctttggtctgtaggctctccaccctgagcacaacttctttacctacaaagacctttactcaacgtatcctatcagctttctttaggctgcaacctttaacagtaaaatacactgcagaatgaacccaaggggttaaaggactttctgttgcttaaggttaaaattagctttgttatgagccatagttagttgtcctaggtattagctccaggacccctgtgtgtctgagtcctcctgaggcccaaggcaggtctattaaagcagcatagcactgcatgtaaccttgcacattgtaagtatatgctgtgtggctgatggaatagtaacatggaaaaagctgtaccctgtgagagtacaattcttcagatgcctccagtgtgtggtccctttattttttttccttttctagcctaggaatattagaaaaataacttgcaaagattatttactgaacttgtagatagctcagccattaaagtacttgctgcaccaagctgtgtggtattaggtgtggtgcagtgagactgtgtggtattaggtgtggggcagtgagactgtgtggtattaggtgtggtgcagtgagactgtgtggtattagatgtggtgcagtgagactgtgtggtattaggtgtggtgcagtgagactgtgtggtattagatgtggtgcagtgagactgtgtggtattaggtgtggggcagtgagactgtgtggtattaggtgtggggcagtgagactgtgtggtattaggtgtggtgcagtgagactgtgtggtattaggtgtggggcagtgagactgtgtggtattaggtgtggtgcagtgagactgtgtggtattaggtgtggggcagtgagactgtgtggtattaggtgtggggcagcgagactgggcgtcggaaacatgaggatgcctgaccagttagcctagccagtgagcttcaggtctcaatgagagaacctttctcaacaacaaggaggcttctgagaacaacccctaggcttaacttctagaacatgtgcatacatgtatgcgcacacacccaggacacatacacacacatccatacacactcataaaacacacagacacacgcccctacagatatatacaccatacagatacactcacacagtcacatatgcatgcccaacacccgtacacacaaatgcatgcatacatgcacatatacatacatacacacacatacacacacacatgctaaactttgccctcacaaacactgtttttgacctttttttagcatgacaataagttgtccaaatatcttcattcacacagaaaattggagtgtaagacacggccaggggtaaggaagtcagtcctttacatagtatttaggtgacaggtacattgttgcccacccaaaacccccaaatgaggccacatcgagactttgaatctgtactgtttgacactctagcatgccttgatctgacttactaattgtgtgctcctccttctaggtcatggtcctggctctgatgaccgttgagctctttggcatgatgggcctcattgggatcaagctgagtgctgtgcctgtggtcatcctgattgcatctgttggcatcggagtggagttcaccgtccacgtggctttggtatgggaggtccttcagggaaactggctcacactgacagagtagaggagtctgggtctctcgtttccataagtcacagggacagcttcaagcacagttttcccaaggaagtttcaaatgtttctgatgggagacaggggtggggtaagaaggtctgtgttgcacacactgcagtgtattagagaagcctgccgtaagggcctctgcacaaaggtgttgctttgtgactaatagcactaagttccaaagttgccaccattgtcccaggacattctatgtgtttaggcaaacatccacactcttttaaatgtagtcagtcataaagtgaggctaaagcaagagctccctcagaggcttggtggggatagcataattgagggtgaagtagctggcagccacagaaatgccctcaggcttgtgaatgaaagctcgggtgcacagccctgaagaacctgctcttccaccgcagcccacccagctctggggaccccccagcttctgcatgcatccatggtggcagaaaacaagaaaaccagggatgctttcaccaggatgtaggcccctctctatatatacactgcctggttgttggaagaagttaactggaaagagagtctagaatccaaaagatgagctggttgttttacacactgagaaaactacttaaaaaccttgtagcattgtgtggtttagtttctgccgacagatgtactcatagtgttaagtacccagggctgttaacaggcatttgataaagagtattgactgaataaatgatagtgtttggaatacccaagttgtgcctgaacccagggatgctaccattgcctttccagcattgtgctttgtgtgtagcacactgtatgtgcattctgtcgcctctcagagctgtcaccgctctcaccagctgtggatgtggatgtacagaacgtgttttgaagctattagtaagatgatgacttagaaacagtatctcagacgtgtccgtgtatgtgagttgcctgtgtgtctgcaagcaaccgagtgagatgcctgtggagactggaaagtagcagatactgttactgaggcaccatttgccttggggattttttgcacagattctcttactggcctggagcacactaagtaggttaggctgactgataggcaaagtccagagatctgcctgttgggattataagcctagaccgtgctttgggttttgttcaaaatatgcgttatcattaactatggctcagtactatgaacagggcattgtaatgaatccagattcagtgagaacaaataaacattgtaggagctaagcagtagactattgaacatagatttggggggaagaaaaggcagacgcagtgtctaatatctccgccagagctgaggctggaatgtaagctcattggtagagggcttggctagctagcataagctcaaaaccaccttctactccagctccagtggatctaaggccctcttatctcctgtgggccccaggcaagcacatggtgaacagacgtatgcacatacatacgagcaaaacacctatacacactcttattttcagaagacctaataaatgatgttttatcgtgacattaaaaagtcagtcatccttttggctttattgctacattttggatgattttttttctccttaatgatagagaagtctctttgagatggctctgtccacctacagaaacttaagaattacttaacttgtagggtaggggctgggctataggtaaggcggcaatgtgcatgcctagtctgcaggatgccctgggctctatcctcagcaccacagaaaaccaggcatagtagtgcacacctgtcaacgtcggccctccagaaatagaacaaggaggctttagctcaaggccattcttgtacagtagtgagtttgagaacggtctgagggacataagaaaagtcaacaaaacccaagtatagacacagctgagcactttaccacacccagaatgtgaccgtgtcaattttctcctttgttcaggcctttctgacagccattggggacaagaaccacagggctatgctcgctctggagcacatgtttgctcccgttctggacggtgctgtgtccactctgctgggtgtactgatgcttgcagggtccgaatttgatttcattgtcaggtaagctgaaatgggagaggacgcttgatacaaaaatcgttattgtcttcgtggcttattccttttctgtcttgtagggggtgtatctcagcctgtctcttacaggcatcttggtctagcagtctcttgggttagagtagaggcaactccacagcttgaaagagcatgtgttgagattccagttgcaaacatctacagagagatgactttggcttctggtaattagtgacaatcagctctaggcccctctccttcagaactgtggaggggacagagatgtacccctttggggtgctaggtatgcgtgaacccttgaatcccatctccaatatcatgtgaaacaagtgcaggtggcttatctctgtaatcccaacccttggctatattgagtttgaggatagccagggatacttaagagcctgccttaaagcaagacagaaagaaaacattgtctacctccaatatggtcacctagctcaagtacacataggaaatgatacagtgtacccagattttgtgtttgggtcccttctgaggcttcataaatgctgggtacctgactgcagggtcaaatttagagactctaagcttggcctgtgtactcctcttggcatcacaaggccagattttgttcctttgggtaagatgctgactttgcaaagtaaaggacagtgagttttcattttggaaactcttaaccacccgagaaagaagcaactctaaggcaatgttggcttccccgggtgaccactgattaaaaccatgacccactggggaggaaaatccagtgttttaagatactagtatgtctttagaatagcaagaggctgtgtacgatgctccatgtctggagaggtgacctaatcaggctgtccttggtagcattgtttagttttgcttttattggtactctgtgaaataggatcactctgcccttccctggtggggctagaactcctgtaccttctggctactcttcaggctacttttttaggaaaagcactagtgtctattagaaagcaggaaagtgccaggcgtggtggcacacacccttaatcctagcacttgggaggcagaggcaggcgcatttctgagttcaaggccagcctggtctacagagtgaattccaggacagccaggactacacagagaaaccctgtctcgaaaaaccaaaaaaaaaaaaagttaaaaagcaggaaagtgtgaagaagacaagaaaatactttttagggggttgggtgttttgggttttgtttgtgtgtttacaaaacaggtctcatgtagcccaggcttgcctcaaactcccatagtagagaacagctttgagttgctgtcccccctccaccttctaagtgctaaaattataggcatgtgctaccacatctggcactaatcaagctttacatgtgtgtacacacaggctggccaagtagggtaagttcacattttcaaataagaaaaggatttttagctacaagttttagccgaatgcatggaataagtgaattcagtcccccacgaggagaggaggaaaaagaaagggtgtgagttcattgaccagccttgaggcattgccaagcttctggcatgtcggtctttctgatggagggcatggttggctcattgagagtagagtaggcagaagattgactccatttcagtgtggactagaactcctgtataatatgctccctcatcatctggggctattaagatacatattgatggaggcctttccagacagctcccaggtgagcctgagcatgctagtattcacacagcctattggtaagttggtgaaccactcattagattctcatttgccaaataccattgagatagttgctcttagatcctggtttgggattattttgttttaaagtgttgatttggtgtttgcttctcccacacactcttacacagatactagctttgtcttaatgatatcaatgcccaatgccaaatgagactgaataggtgtcgtctcttggctctgtcaacacaaaggtaatgctggcctcacaggtgcctttttttgtgattactggcaggtagcaggaaacccatctctgctttgtggttatagtctgcagggattgggagatatagctctctaagcagttcaaagccgagagaaagttgggtgtgcggagaccttgcaggtcaagtcagctgaggagctggagcacgggtttacataggatactaaggaacacaacctaaccgctcatcctgtcctcgctgggcctgctcattccctcccagagagatcatggtttctgtttcaggcacttagaagggcttcccattttctaaaataggatggataaagtcctcactggagttgaataaatgagcagactctgtatacatgaagccgtcagaaatctaattggtctgtttatgttgcagctagatggatgggatgcaaatgtcttaatgcctgccgttgatatatgcctccaccaaaagttccacagtttatgttagagcaagacattggcagcttgggtttttactaatgcaactcagcaggttttagtgtttacaatagctgtaaggtatttgtatttttatagggattctctctcctttcatctaatgtcaaaacatgcgtagacatggcattgtaggggagctgttcagttttatgatccaggggaccattaacagatagatatctagccgtccctctctctttccaggacatcctaaccccagcccccagcccccacctttccatcaaagccctagaaatcattagttccagaaaagcatccttgcgtggagtgtgcgccctccagctgtgcagagtgctgaccagaggcgggccctggctgacaggtttaccttggtattcttgaaggatcccagacctttagccctcaatggtgggaacgcagaactcactgttcactctccctcttctaccatggtgcttcagcctttatgtttgaattggctgacgcaaaacaagagtattcaaagatgctaaattgttcaacaaaagattggcctgcagagcagccctgccattgtggagtgccctcgccctgcacaatgccccggctctcacagaatgggcagcgttttctttgcagaaagctggttcctgtgttctaaaaaggttgcaattggagctgtttggtaaaactggaacccagagaaattctaggcagaaagttgtgcttaatgaaagcccagggaaagccttacctgatgcactagagacttaattagagattctgctgtaagaggcaggggcatttcacagtggattagctgcctgcagaagtgggctgcttttcctcctgcgccaaattgtttaaattctacgccctgtacttcatttgtttgcttctgctgggttctcgagaggggcccctcgcccagcattgtatctgtactttcagggtgctcagccattcagctcacattccatattcctgtgttggtgtaacacgttcataaataacatcacaggcaggagatttcatcaaattaacaattttttccccttcccttcatgaagtaacgctgcagggtgtggaggaatctagtcctcagagccgcaaggataaataaaaaaaaaaaaagagcaaaatcccaaactaatctatgctaagtttgaactagtgttttgtggttgacttctcagcccagcaggtttgttgagctgagctcttagtgcagagtaacctttgggcgcccgggggcgtggaatgctctccttcctcgctgacatgtcccatatcccttttgtcttccagatacttctttgccgtcctggccattctcaccgtcttgggggttctcaatggactggttctgctgcctgtcctcttatccttctttggaccgtgtcctgaggtcagtgattgccccccaatgccaccccggattgagatattcaggtgttgccatattagcagatggtctgggcttttcggtcaacatccattaacaatggctgtttcatttttccctttgactgttttacaccaatggatgggcagcttaggaaggttttcgacctcagataactgtttgggcttaacaggacagacaagataggtagccgatgtctggcttctctggtattagaagttgggacaatgtttttgtgccagaatcgtgagtgtcttccagatagcagggagacgggaagtaaatacagtttaatgtatggggttcccctacgaggtcacagaggccaataatcctgggctttgaactttgaaagtgacctgcgggtcaacaaacgtaacatttttaattggaaacctccttcaacaggtgtctccagccaatggcctaaaccgactgcccactccttcgcctgagccgcctccaagtgtcgtccggtttgccgtgcctcctggtcacacgaacaatgggtctgattcctccgactcggagtacagctctcagaccacggtgtctggcatcagtgaggagctcaggcaatacgaagcacagcagggtgccggaggccctgcccaccaagtgattgtggaagccacagaaaaccctgtctttgcccggtccactgtgagtcacctggggggttggcaagggggggggggcggtggggaagattcatttgtcctgtggcagtgggccaggaggttcggagcaggctcttgtgtactctgagcagcagatctagtagacagatggggtgcaagaaaaaagtgtacacgccggggtcaaatcacaggaaaccaccagcgcgccattctttgtagactgtaggatgtgtttccttttaatcttgtggactgaaccggggcagtgccagcattgttacaaggaggcatagtttagtggtgaagcgcttgcctggaattcccaaagcccaggggctcatccccagccctacccttattcagaatggactcagctttttagggcctttatctccttttgccctatctgtcctttcagaaggacatgtgaggagatgatgtaacaaacagcgtagtattccaccacaagaagcatttattcttttcctctcgtctctggtacctttaagtggccttggcacaaagttatttaattactatctaaggctttcccgtagctttgtggcttcagggtcttattgttgtagatggctggagatctattgaaagtatgcagacgcatcctgagtacttaattccagaaggtagtgaggagtccacaggttccttccttacccgcacccccaccctccaagtcaagatgctccaccttgaagagttaaggaaggctccaaaactctggcttgcctgaggcccccaccatgtttacaggaaatgtgtattccagtcccaggctagagcagccttccttgaaggagagaagggctggccagtgaattgtgtccccgtcctctgtccaccccccccccccaacacacacacacacacacacacacacacacacacacacccgtgacgatgccctgctgagctgtagtgtgaatctggttctgttcagcaggagcctaaaaataggtacaagctcaccgactttgaagttggtcacggtccttttgattgatgtggccactgtgttgctccgagctttgtacagaggacccagaaaccagattcctaatagtcaggcagagatgtattgcctaacagaactatctgtgtggaagtttttgctctgctcttacagtacatttgcttctggccacacctggcttagggctggggacaggtgaaaacaacactaatatgacccagaaacaggagccttatacttctaaaagctactatttatggttgcatatttccaagacacccaggctcttggctatccaggccttggcagttttgaggcagagaacttaggcctgggggcagttggttgtgagggtaagttgagatctatgggcagtggctatatgcctccagccagctgtgcaggtgcaggcaggattcacatcctggaggtttctaaaggaataggttttcttttgtgggtggaacatcaccaccatcctacctccctgtgttgacctatgtctctcctctgcaggtggtccatccggactccagacatcagcctcccttgacccctcggcaacagccccacctggactctggctccttgtcccctggacggcaaggccagcagcctcgaagggatccccctagagaaggcttgcggccacccccctacagaccgcgcagagacgcttttgaaatttctactgaagggcattctggccctagcaatagggaccgctcagggccccgtggggcccgttctcacaaccctcggaacccaacgtccaccgccatgggcagctctgtgcccagctactgccagcccatcaccactgtgacggcttctgcttcggtgactgttgctgtgcatcccccgcctggacctgggcgcaacccccgaggggggccctgtccaggctatgagagctaccctgagactgatcacggggtatttgaggatcctcatgtgccttttcatgtcaggtgtgagaggagggactcaaaggtggaggtcatagagctacaggacgtggaatgtgaggagaggccgtgggggagcagctccaactga